(19) United States
(12) Patent Application Publication
(10) Pub. No.: US 2023/0212582 A1
(43) Pub. Date: Jul. 6, 2023

(54) COMPOSITIONS OF SMAD7 ANTISENSE OLIGONUCLEOTIDES (ASO) AND METHODS OF USING THE SAME

(71) Applicant: Nogra Pharma Limited, Dublin 2 (IE)

(72) Inventors: Francesca Viti, Salorino (CH); Giovanni Monteleone, Grottaferrata (IT); Salvatore Demartis, Milan (IT); Salvatore Bellinvia, Mendrisio (CH); Marie McNulty, Dublin (IE)

(21) Appl. No.: 17/996,505

(22) PCT Filed: Apr. 23, 2021
(86) PCT No.: PCT/IB2021/000272

§ 371 Date: Oct. 18, 2022

Related U.S. Application Data
(60) Provisional application No. 63/135,283, filed on Jan. 8, 2021.
(60) Provisional application No. 63/030,818, filed on May 27, 2020.
(60) Provisional application No. 63/015,120, filed on Apr. 24, 2020.

(51) Int. Cl. C12N 15/113; G01N 33/68; A61P 1/12

(52) U.S. Cl. CPC C12N 15/1136; G01N 33/6863; A61P 1/12; C12N 2310/3341; C12N 2310/315; C12N 2310/322

(57) ABSTRACT

The present disclosure relates to compositions of oligonucleotide (e.g., SMAD7 antisense oligonucleotide diastereomers) and methods of manufacturing, assessing efficacy, and using the compositions.

### RELATED APPLICATIONS

The present application claims priority to U.S. Provisional Patent Application No. 63/015,120, filed on Apr. 24, 2020; U.S. Provisional Patent Application No. 63/030,818, filed on May 27, 2020; and U.S. Provisional Patent Application No. 63/135,283, filed on Jan. 8, 2021, the entire contents of each of which are hereby incorporated by reference for all purposes.

### SEQUENCE LISTING

The present specification makes reference to a Sequence Listing, submitted electronically as a .txt file named “GIU_061_Sequence_Listing.txt” on Apr. 23, 2021. The .txt file was generated on Mar. 24, 2021 and is 3.711 bytes in size. The entire contents of the Sequence Listing are herein incorporated by reference.

### BACKGROUND

Non-superimposability of an object onto its mirror image is called chirality. A molecule is chiral if it cannot be superimposed onto its mirror image. A chiral center of a molecule is an atom that has four different atoms or groups bonded to it in such a manner that it has a nonsuperimposable mirror image.

Stereoisomers are molecules that differ in spatial arrangement of atoms. Enantiomers are a pair of stereoisomers that are mirror images of each other that are nonsuperimposable. Diastereomers are non-mirror image non-identical stereoisomers.

Enantiomers often have identical chemical and physical properties, except for the direction in which they rotate polarized light and how they interact with different chiral entities, such as chiral receptors, chiral solvents, chiral chromatographic stationary phases, and electromagnetic radiation. On the other hand, diastereomers display different chemical and physical properties and are typically considered different chemical and pharmacological entities.

Many biological molecules and components of biological systems (e.g., receptors, enzymes, and co-factors) are chiral, and different stereoisomers can have different effects on biological organisms. The chirality of a drug can influence all aspects of pharmacological and pharmacokinetic activities, e.g., bioavailability, distribution, metabolism, and permeability. For example, while one of a drug's enantiomers may have favorable physiological and/or clinical effects, the other enantiomer may be less active or inactive, have different physiological and/or clinical activities, or produce adverse effects.

Oligonucleotides are short DNA or RNA molecules that have a wide range of applications in therapeutics (e.g., antisense therapies), genetic testing, and forensics. Oligonucleotides are chemically synthesized using nucleotide building blocks. To promote the stability of oligonucleotides, chemical modifications, such as phosphodiester backbone modifications and sugar ring modifications have been employed. Phosphorothioate (PS) modification of the backbone is a common practice to protect synthetic oligonucleotides from degradation, thereby improving the stability and pharmacological profiles of the oligonucleotides. PS modification converts the locally achiral phosphor atom of the phosphodiester (PO) linkage into a chiral PS center, resulting in diastereomers of the synthetic oligonucleotides. However, common synthetic methods of PS modified oligonucleotides do not allow for control of the chirality at the phosphor atom. Meanwhile, individual diastereomer levels in a given synthetic batch of PS oligonucleotides cannot be analytically determined without chirality-at-phosphor control.

While how controlling chirality of the PS linkage modulates therapeutic profiles of oligonucleotides is still under debate, recent studies suggest that stereodefined systems may have improved therapeutic efficacy. Techniques are described in the art for the evaluation of stereoisomeric purity, e.g., optical rotation or polarimetry and nuclear magnetic resonance (NMR) spectroscopy.

There remains a need for improved strategies for evaluating the diastereomeric profile of PS oligonucleotides and for predicting their related pharmacological and/or clinical efficacy. There further remains the need to provide oligonucleotides with defined diastereomeric profiles having known pharmacological and/or clinical efficacy. The present disclosure provides oligonucleotides having characterized diastereomeric profiles and improved strategies for assessing and predicting the pharmacological and/or clinical efficacy of the same using diastereomeric profiles.

Crohn's disease (CD) is a chronic inflammatory disorder characterized by transmural and segmental lesions. CD can occur in any part of the alimentary tract, although the terminal ileum and the right colon are the most commonly involved sites. Nearly half of CD patients require surgery within 10 years from diagnosis. Bowel resection (e.g., ileocecal resection) is the most common surgical procedure performed for CD. However, surgery does not cure CD. While surgery often results in clinical remission, most patients ultimately relapse and require subsequent surgery. Postoperative recurrence of CD can be diagnosed by histologic or endoscopic findings or by the presence of clinical symptoms.

There remains a need for strategies for predicting, treating and preventing postoperative recurrence of CD. The present disclosure provides methods for predicting, treating, and/or preventing postoperative recurrence of CD and for identifying patients at risk of postoperative recurrence of CD.

### SUMMARY

The present application provides pharmaceutical compositions of SMAD7 antisense oligonucleotide diastereomers and methods of manufacturing, assessing efficacy, and using the compositions.

Accordingly, one aspect of the present application provides a pharmaceutical composition, comprising a plurality of diastereomers of a SMAD7 antisense oligonucleotide (ASO) having a sequence according to SEQ ID NO: 1 (5′-GTXGCCCCTTCTCCCXGCAGC-3′) in which all internucleoside linkages are O,O-linked phosphorothioate linkages and X is 5-methyl 2′-deoxycytidine, wherein the plurality of diastereomers has a phosphorus-31 nuclear magnetic resonance (³¹P-NMR) spectrum comprising: a) one or more resonances between 54.8 to 55.5 ppm; and b) a first principal component 1 (PC1) score and a second principal component 2 (PC2) score, wherein: (i) the PC1 score is below about −0.20 or above about 0.25 and the PC2 score is below about 0.00; or (ii) the PC1 score is outside of the range of −0.32 to 0.30 and/or the PC2 score that is outside the range of 0.00 to 0.20.

In some embodiments, the ³¹P-NMR spectrum comprises a PC1 score that is below about −0.20 and a PC2 score that is below about 0.00. In some embodiments, the ³¹P-NMR spectrum comprises a PC1 score that is from about −0.47 to about −0.20 and a PC2 score that is from about −0.27 to about 0.00. In some embodiments, the ³¹P-NMR spectrum comprises a PC1 score that is above about 0.25 and a PC2 score that is below about 0.00. In some embodiments, the ³¹P-NMR spectrum comprises a PC1 score that is from about 0.65 to about 0.9 and a PC2 score that is from about −0.47 to about 0.00. In some embodiments, the ³¹P-NMR spectrum comprises a PC1 score that is outside of the range of −0.32 to 0.30 when the PC2 score is above 0.00 and below 0.20. In some embodiments, the ³¹P-NMR spectrum comprises a PC2 score is outside the range of 0.00 to 0.20 when the PC1 is above −0.32 and below 0.30.

In some embodiments, the ³¹P-NMR spectrum comprises two or more resonances between 54.8 to 55.5 ppm. In some embodiments, the two or more resonances between 54.8 to 55.5 ppm have different intensities. In some embodiments, the ³¹P-NMR spectrum comprises, independently at about 54.8 ppm, at about 54.9 ppm, at about 55.0 ppm, at about 55.1 ppm, at about 55.2 ppm, at about 55.3 ppm, at about 55.4 ppm, and/or at about 55.5 ppm, one or more resonances. In some embodiments, the ³¹P-NMR spectrum comprises, independently at about 54.8 ppm, at about 54.9 ppm, at about 55.0 ppm, at about 55.1 ppm, at about 55.2 ppm, at about 55.3 ppm, at about 55.4 ppm, and/or at about 55.5 ppm, two or more resonances.

In some embodiments, the pharmaceutical composition comprises 100 millimoles to 5 moles of SMAD7 ASO. In some embodiments, the pharmaceutical composition comprises 100 millimoles to 2 moles, 100 millimoles to 900 millimoles, 300 millimoles to 5 moles, 300 millimoles to 2 moles, 300 millimoles to 900 millimoles, 900 millimoles to 5 moles, or 900 millimoles to 2 moles of SMAD7 ASO. In some embodiments, the pharmaceutical composition comprises about 300 millimoles of SMAD7 ASO. In some embodiments, the pharmaceutical composition comprises about 900 millimoles of SMAD7 ASO. In some embodiments, the pharmaceutical composition comprises about 2 moles of SMAD7 ASO. In some embodiments, the pharmaceutical composition comprises greater than 300 millimoles, greater than 900 millimoles, or greater than 2 moles of SMAD7 ASO.

In some embodiments, the pharmaceutical composition comprises: a) about 0.5% to about 30% by weight of the SMAD7 ASO; b) about 20% to about 50% by weight mannitol; c) about 10% to about 30% by weight microcrystalline cellulose; and d) an enteric coating comprising an ethylacrylate-methacrylic acid copolymer. In some embodiments, the pharmaceutical composition comprises: a) an intra-granular phase comprising: i) about 5% to about 10% by weight of the SMAD7 ASO; ii) about 40% by weight mannitol; iii) about 8% by weight microcrystalline cellulose; iv) about 5% by weight hydroxypropyl methylcellulose; and v) about 2% by weight sodium starch glycolate; b) an extra-granular phase comprising: i) about 17% by weight microcrystalline cellulose; ii) about 2% by weight sodium starch glycolate; iii) about 0.4% by weight magnesium stearate; and c) an enteric coating comprising an ethylacrylate-methacrylic acid copolymer, wherein the percentages by weight are the weights of the ingredients compared to the total weight of the pharmaceutical composition.

In some embodiments, the pharmaceutical composition comprises: a) about 5% to about 30% by weight of the SMAD7 ASO; b) about 20% to about 50% by weight mannitol; c) about 10% to about 30% by weight microcrystalline cellulose; d) about 0.5% to about 10% by weight hydroxypropyl methylcellulose; e) about 0.5% to about 10% by weight sodium starch glycolate; f) about 0.05% to about 1% by weight magnesium stearate; g) about 0.5% to about 10% by weight Opadry® AMB; and h) about 5% to about 20% by weight Acryl-EZE®. In some embodiments, the pharmaceutical composition comprises: a) about 8.5% by weight of the SMAD7 ASO; b) about 40% by weight mannitol; c) about 25% by weight microcrystalline cellulose; d) about 5% by weight hydroxypropyl methylcellulose; e) about 4% by weight sodium starch glycolate; f) about 0.4% by weight magnesium stearate; g) about 4% by weight Opadry® AMB; and h) about 10% to about 15% by weight Acryl-EZE®. In some embodiments, the pharmaceutical composition comprises: a) about 23% by weight of the SMAD7 ASO; b) about 28% by weight mannitol; c) about 25% by weight microcrystalline cellulose; d) about 5% by weight hydroxypropyl methylcellulose; e) about 4% by weight sodium starch glycolate; f) about 0.4% by weight magnesium stearate; g) about 4% by weight Opadry® AMB; and h) about 7% to about 12% by weight Acryl-EZE®.

In some embodiments, the pharmaceutical composition is formulated as a tablet. In some embodiments, the pharmaceutical composition is for oral administration. In some embodiments, the pharmaceutical composition is for oral administration wherein the SMAD7 ASO is administered to a subject in need thereof at a dose of about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg. In some embodiments, the pharmaceutical composition is for oral administration about every 6 hours, about every 12 hours, about every 24 hours, about every 48 hours, about every 72 hours, every day, two-times a week, once in 2 weeks, or once a month. In some embodiments, the pharmaceutical composition is for oral administration at a dose of about 160 mg daily.

In some embodiments, the pharmaceutical composition down-regulates SMAD7 mRNA and/or protein in a cell. In some embodiments, the down-regulation of SMAD7 protein expression is more than 40% compared to an untreated cell. In some embodiments, the pharmaceutical composition is clinically efficacious in treating or preventing inflammatory bowel disease. In some embodiments, the inflammatory bowel disease is Crohn's disease. In some embodiments, the Crohn's disease is postoperative recurrence of Crohn's disease.

In one aspect, the present disclosure provides a method of treating or preventing an inflammatory bowel disease in a subject in need thereof, comprising administering to the subject a pharmaceutical composition provided herein. In one aspect, the present disclosure provides a use of a pharmaceutical composition provided herein for the manufacture of a medicament for the treatment of an inflammatory bowel disease. In one aspect, the present disclosure provides a pharmaceutical composition provided herein for use in the treatment of an inflammatory bowel disease. In some embodiments, the inflammatory bowel disease is Crohn's disease. pharmaceutical composition, the Crohn's disease is postoperative recurrence of Crohn's disease

In another aspect, the present disclosure provides a method of preventing or treating postoperative recurrence of Crohn's disease (CD) in a subject in need thereof, comprising inhibiting SMAD7 in the subject. In some embodiments, at least one sample from the subject has an elevated SMAD7 level relative to a known control level, wherein the known control level is the SMAD7 level in a sample collected from the subject prior to or during the surgical treatment for CD or the SMAD7 level in a sample collected from a healthy subject without CD.

In another aspect, the present disclosure provides a method for preventing or treating postoperative recurrence of Crohn's disease (CD) in a subject in need thereof, comprising preventing a Crohn's Disease Activity Index (CDAI) score greater than 200 of the subject or reducing CDAI score for at least 50 points. In some embodiments, the method comprises preventing a CDAI score greater than 150. In some embodiments, the method comprises reducing CDAI score for at least 100 points.

In another aspect, the present disclosure provides a method for predicting postoperative recurrence of Crohn's disease (CD) in a subject in need thereof, comprising determining the level of SMAD7 in a first sample from the subject, wherein an elevated SMAD7 level relative to a known control level is predictive of the recurrence of CD, wherein the known control level is the SMAD7 level in a sample collected from the subject prior to or during the surgical treatment for CD or the SMAD7 level in a sample collected from a healthy subject without CD.

In another aspect, the present disclosure provides a method of identifying a subject at risk of postoperative recurrence of Crohn's disease (CD), comprising: determining the level of SMAD7 in a first sample from the subject, wherein an elevated SMAD7 level relative to a known control level identifies the subject as being at risk for the recurrence of CD, wherein the known control level is the SMAD7 level in a sample collected from the subject prior to or during the surgical treatment for CD or the SMAD7 level in a sample collected from a healthy subject without CD.

In some embodiments, if the SMAD7 level is elevated relative to the known control level, then administering to the subject a pharmaceutical composition comprising a SMAD7 ASO; or if the SMAD7 level is not elevated relative to the known control level, then determining the level of SMAD7 in a second sample from the subject.

In some embodiments, the second sample is collected immediately after, about 1 hour after, about 3 hours after, about 6 hours after, about 12 hours after, about 1 day after, about 3 days after, about 1 week after, about 2 weeks after, about 1 month after, about 2 months after, about 3 months after, about 4 months after, about 5 months after, about 6 months after, about 7 months after, about 8 months after, about 9 months after, about 10 months after, about 11 months after, or about 12 months after the first sample.

In another aspect, the present disclosure provides a method for preventing or treating postoperative recurrence of Crohn's disease (CD) in a subject in need thereof, comprising administering to the subject a pharmaceutical composition comprising a SMAD7 ASO.

In some embodiments, inhibiting SMAD7 in the subject comprises administering to the subject a pharmaceutical composition comprising a SMAD7 ASO. In some embodiments, the SMAD7 ASO is administered at a dose of about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg. In some embodiments, the SMAD7 ASO is administered about every 6 hours, about every 12 hours, about every 24 hours, about every 48 hours, about every 72 hours, every day, two-times a week, once in 2 weeks, or once a month. In some embodiments, the SMAD7 ASO is administered at a dose of about 160 mg daily.

In some embodiments, the SMAD7 ASO comprises a sequence selected from any one of SEQ ID NOs: 1-6 or a pharmaceutically acceptable salt thereof. In some embodiments, the SMAD7 ASO has a sequence according to SEQ ID NO: 1 (5′-GTXGCCCCTTCTCCCXGCAGC-3′) in which all internucleoside linkages are O,O-linked phosphorothioate linkages and X is 5-methyl 2′-deoxycytidine. In some embodiments, the pharmaceutical composition comprises a plurality of diastereomers of a SMAD7 ASO having a sequence according to SEQ ID NO: 1 (5′-GTXGCCCCTTCTCCCXGCAGC-3′) in which all internucleoside linkages are O,O-linked phosphorothioate linkages and X is 5-methyl 2′-deoxycytidine, wherein the plurality of diastereomers has a ³¹P-NMR spectrum comprising: a) one or more resonances between 54.8 to 55.5 ppm; and b) a first principal component 1 (PC1) score and a second principal component 2 (PC2) score, wherein: (i) the PC1 score is below about −0.20 or above about 0.25 and the PC2 score is below about 0.00; or (ii) the PC1 score is outside of the range of −0.32 to 0.30 and/or the PC2 score is outside the range of 0.00 to 0.20. In some embodiments, the ³¹P-NMR spectrum comprises: a) a PC1 score below about −0.20 and a PC2 score below about 0.00; b) a PC1 score above about 0.25 and a PC2 score below about 0.00; c) a PC1 score from about −0.47 to about −0.20 and a PC2 score from about −0.27 to about 0.00; d) a PC1 score from about 0.65 to about 0.9 and a PC2 score from about −0.47 to about 0.00; e) a PC1 score outside of the range of −0.32 to 0.30 when the PC2 score is above 0.00 and below 0.20; and/or; and/or f) a PC2 score outside the range of 0.00 to 0.20 when the PC1 is above −0.32 and below 0.30.

In some embodiments, the postoperative recurrence is selected from the group consisting of: endoscopic recurrence, histological recurrence, radiographic recurrence, clinical recurrence, and combinations thereof.

In some embodiments, the subject has received at least one surgical treatment for CD. In some embodiments, the at least one surgical treatment is selected from a group consisting of: bowel resection, ileocolonic resection, colectomy, proctocolectomy, strictureplasty, ileostomy, anal fistulotomy, and combinations thereof. In some embodiments, the at least one surgical treatment is ileocolonic resection.

In some embodiments, the subject exhibits no sign of postoperative recurrence. In some embodiments, the subject exhibits at least one sign of postoperative recurrence. In some embodiments, the subject exhibits at least one sign of endoscopic recurrence. In some embodiments, the at least one sign is a mucosal lesion.

In some embodiments, the sample is a mucosal sample. In some embodiments, the sample is collected immediately after, about 1 hour after, about 3 hours after, about 6 hours after, about 12 hours after, about 1 day after, about 3 days after, about 1 week after, about 2 weeks after, about 1 month after, about 2 months after, about 3 months after, about 4 months after, about 5 months after, about 6 months after, about 7 months after, about 8 months after, about 9 months after, about 10 months after, about 11 months after, or about 12 months after a surgical treatment for CD.

In some embodiments, the SMAD7 level is SMAD7 mRNA level or SMAD7 protein level. In some embodiments, the elevated SMAD7 level is at least about 10%, at least about 20%, at least about 30%, at least about 40%, at least about 60%, or at least about 80% elevated as compared to the known control level.

In some embodiments, the pharmaceutical composition is administered orally. In some embodiments, the pharmaceutical composition is administered immediately after, about 1 hour after, about 3 hours after, about 6 hours after, about 12 hours after, about 1 day after, about 3 days after, about 1 week after, about 2 weeks after, about 1 month after, about 2 months after, about 3 months after, about 4 months after, about 5 months after, about 6 months after, about 7 months after, about 8 months after, about 9 months after, about 10 months after, about 11 months after, or about 12 months after a surgical treatment for CD.

In another aspect, the present disclosure provides a pharmaceutical composition comprising a plurality of diastereomers of a SMAD7 antisense oligonucleotide (ASO) that is pharmacologically and/or clinically efficacious, made by a method comprising: a) confirming a ³¹P-NMR spectrum for the plurality of diastereomers of one or more resonances between 54.8 to 55.5 ppm; and b) confirming the ³¹P-NMR spectrum has: (i) a first principal component 1 (PC1) score that is below about −0.20 or above about 0.25 and a second principal component 2 (PC2) score that is below about 0.00, or (ii) a PC1 score that is outside of the range of −0.32 to 0.30 and/or a PC2 score that is outside the range of 0.00 to 0.20, as determined by principal component analysis (PCA).

In another aspect, the present disclosure provides a method of manufacturing a pharmaceutical composition comprising a plurality of diastereomers of a SMAD7 antisense oligonucleotide (ASO), the method comprising: a) confirming a ³¹P-NMR spectrum for the plurality of diastereomers of one or more resonances between 54.8 to 55.5 ppm; and b) confirming the ³¹P-NMR spectrum has: (i) a first principal component 1 (PC1) score that is below about −0.20 or above about 0.25 and a second principal component 2 (PC2) score that is below about 0.00, or (ii) a PC1 score that is outside of the range of −0.32 to 0.30 and/or a PC2 score that is outside the range of 0.00 to 0.20, as determined by principal component analysis (PCA).

In some embodiments, the method comprises confirming: a) the PC1 score is below about −0.20 and the PC2 score is below about 0.00; b) the PC1 score is above about 0.25 and the PC2 score is below about 0.00; c) the PC1 score is from about −0.47 to about −0.20 and the PC2 score is from about −0.27 to about 0.00; d) the PC1 score is from about 0.65 to about 0.9 and the PC2 score is from about −0.47 to about 0.00; e) the PC1 score is outside of the range of −0.32 to 0.30 when the PC2 score is above 0.00 and below 0.20; and/or f) the PC2 score is outside the range of 0.00 to 0.20 when the PC1 is above −0.32 and below 0.30. In some embodiments, the method comprises confirming: a) the 31P-NMR spectrum has two or more resonances between 54.8 to 55.5 ppm; b) the 31P-NMR spectrum has one or more resonances at about 54.8 ppm; c) the 31P-NMR spectrum has two or more resonances at about 54.8 ppm; d) the 31P-NMR spectrum has one or more resonances at about 54.9 ppm; e) the 31P-NMR spectrum has two or more resonances at about 54.9 ppm; f) the 31P-NMR spectrum has one or more resonances at about 55.0 ppm; g) the 31P-NMR spectrum has two or more resonances at about 55.0 ppm; h) the 31P-NMR spectrum has one or more resonances at about 55.1 ppm; i) the 31P-NMR spectrum has two or more resonances at about 55.1 ppm; j) the 31P-NMR spectrum has one or more resonances at about 55.2 ppm; k) the 31P-NMR spectrum has two or more resonances at about 55.2 ppm; 1) the 31P-NMR spectrum has one or more resonances at about 55.3 ppm; m) the 31P-NMR spectrum has two or more resonances at about 55.3 ppm; n) the 31P-NMR spectrum has one or more resonances at about 55.4 ppm; o) the 31P-NMR spectrum has two or more resonances at about 55.4 ppm; p) the 31P-NMR spectrum has one or more resonances at about 55.5 ppm; q) the 31P-NMR spectrum has two or more resonances at about 55.5 ppm; and/or r) the 31P-NMR spectrum has two or more resonances between 54.8 to 55.5 with different intensities.

In another aspect, the present disclosure provides a method of predicting pharmacological and/or clinical efficacy of a candidate composition comprising a plurality of diastereomers of a SMAD7 antisense oligonucleotide (ASO), the method comprising subjecting a ³¹P-NMR spectrum for the plurality of diastereomers to principal component analysis (PCA) to obtain a first principal component (PC1) and a second principal component (PC2), wherein: a) a PC1 score that is below about −0.20 or above about 0.25 and a PC2 score that is below about 0.00 is predictive of pharmacological and/or clinical efficacy, or b) the PC1 score is outside of the range of −0.32 to 0.31 and/or the PC2 score that is outside the range of 0.00 to 0.20, is predictive of pharmacological and/or clinical efficacy. In some embodiments, a) a PC1 score that is below about −0.20 and a PC2 score that is below about 0.00; b) a PC1 score above about 0.25 and a PC2 score that is below about 0.00; c) a PC1 score from about −0.47 to about −0.20 and a PC2 score that is from about −0.27 to about 0.00; d) a PC1 score from about 0.65 to about 0.9 and a PC2 score that is from about −0.47 to about 0.00; e) a PC1 score outside of the range of −0.32 to 0.30 when the PC2 score is above 0.00 and below 0.20; and/or f) the PC2 score is outside the range of 0.00 to 0.20 when the PC1 is above −0.32 and below 0.30, is predictive of pharmacological and/or clinical efficacy.

In some embodiments, the PCA comprises selecting principle components using a modeling data set. In some embodiments, the modeling data set comprises five, six, seven, eight, nine, ten, or up to twenty-seven ³¹P-NMR spectra selected from the ³¹P-NMR spectra as shown in FIGS. 5C-5CC. In some embodiments, the modeling data set comprises the ³¹P-NMR spectra as shown in FIGS. 5C, 5L, 5X, 5H, 5P, 5I, 5M, 5T, 5S, and 5BB. In some embodiments, the principal components are selected to account for more than 90% variance of the modeling data set. In some embodiments, the ³¹P-NMR is performed in solution at about 14.1 T. In some embodiments, the SMAD7 ASO comprises a sequence selected from any one of SEQ ID NOs: 1-6 or a pharmaceutically acceptable salt thereof. In some embodiments, internucleotide linkages of the SMAD7 ASO are O,O-linked phosphorothioates. In some embodiments, the SMAD7 ASO has a sequence according to SEQ ID NO: 1 (5′-GTXGCCCCTICTCCCXGCAGC-3′) in which all internucleoside linkages are O,O-linked phosphorothioate linkages and X is 5-methyl 2′-deoxycytidine.

In some embodiments, provided here is a method of treating an inflammatory bowel disease in a subject in need thereof, comprising administering to the subject a SMAD7 ASO manufactured according a method disclosed herein or a SMAD7 ASO predicted to be pharmacologically and/or clinically efficacious according a method disclosed herein. In some embodiments, the inflammatory bowel disease is Crohn's disease. In some embodiments, the Crohn's disease is postoperative Crohn's disease.

These and other aspects and advantages and other aspects of invention described in the present application are illustrated by the following figures, detailed description, and claims.

### BRIEF DESCRIPTION OF THE DRAWINGS

In order to understand the invention and to demonstrate how it may be carried out in practice, embodiments are now described, by way of non-limiting example only, with reference to the accompanying drawings in which:

FIG. 1 is an illustration showing the structure of exemplary SMAD7 antisense oligonucleotides (ASOs).

FIGS. 2A-2E show Western blot images of whole cell lysates prepared from HCT-116 cells transfected with different batches of SMAD7 ASOs. FIG. 2A shows Western blots probed with SMAD7 (top panel) and β-actin loading control (bottom panel) of lysates prepared from HCT-116 cells transfected with either 0.5 μg/ml or 1 μg/ml of SMAD7 ASO (SEQ ID NO:1) Batches T, P, H, A, or J or Lipofectamine 3000 alone (negative control). FIG. 2B shows Western blots probed with SMAD7 (top panel) and f-actin loading control (bottom panel) of lysates prepared from HCT-116 cells transfected with 1 μg/ml of SMAD7 ASO (SEQ ID NO:1) Batches E, M, X, B, F, S, C, K, W. U, or A or Lipofectamine 3000 alone (negative control). FIG. 2C shows Western blots probed with SMAD7 (top panel) and (i-actin loading control (bottom panel) of lysates prepared from HCT-116 cells transfected with 1 μg/ml of SMAD7 ASO (SEQ ID NO:1) Batches I, V, N, O, Y, Z, D, G, Q, L, or A or Lipofectamine 3000 alone (negative control). FIG. 2D shows Western blots probed with SMAD7 (top panel) and p-actin loading control (bottom panel) of lysates prepared from HCT-116 cells transfected with 1 μg/ml of SMAD7 ASO (SEQ ID NO:1) Batches A, F, O, or S or Lipofectamine 3000 alone (negative control). FIG. 2E shows Western blots probed with SMAD7 (top panel) and β-actin loading control (bottom panel) of lysates prepared from HCT-116 cells transfected with 1 μg/ml of SMAD7 ASO (SEQ ID NO:1) Batches A or S20 or Lipofectamine 3000 alone (negative control).

FIG. 3A is a bar graph showing mean percentage difference in SMAD7 protein expression following transfection of HCT-116 cells with different SMAD7 ASO (SEQ ID NO:1) batches as compared to cells treated with Lipofectamine 3000 alone (negative control).

FIG. 3B is a bar graph showing mean Performance Score of SMAD7 ASO (SEQ ID NO:1) batches relative to Batch A (positive control).

FIG. 3C is a bar graph showing real-time PCR analysis of SMAD7 mRNA expression of HCT-116 cells transfected with SMAD7 ASO (SEQ ID NO:1) Batches A, X, K, or T, as compared to cells treated with Lipofectamine 3000 alone (negative control).

FIG. 4A is a bar graph showing mean changes in Crohn's disease activity index (CDAI) score in patients receiving different batches of SMAD7 ASO compared to baseline at the end of 4 weeks of daily treatment.

FIG. 4B is the superposed ³¹P-NMR spectra at 14.1 T in the region of 56.5-53.5 ppm of the SMAD7 ASO (SEQ ID NO:1) batches used to treat patients for the clinical performance plot in FIG. 4A.

FIG. 5A is the superposed ¹H-NMR spectra of 27 tested batches of SMAD7 ASO (SEQ ID NO:1). FIG. 5B is the superposed ³¹P-NMR spectra at 14.1 T in the region of 57-53 ppm of 27 tested batches of SMAD7 ASO (SEQ ID NO:1). FIGS. 5C, 5D, 5E, 5F, 5G, 5H, 5I, 5J, 5K, 5L, 5L, 5M, 5N, 5O, 5P, 5Q, 5R, 5S, 5T, 5U, 5V, 5W, 5X, 5Y, 5Z, 5AA, 5BB, and 5CC are ³¹P NMR spectra in the region of 56.5-53.5 ppm of SMAD7 ASO (SEQ ID NO:1) Batch A, B, C, D, E, F, G, H, I, J, K, L, M, N, O, P, Q, R, S, T, U, V, W, X, Y, Z, and S20, respectively. FIG. 5DD is superposed ³¹P-NMR spectra in the region of 56.5-53.5 ppm of SMAD7 ASO (SEQ ID NO:1) Batches A (solid line) and Y (dashed line). FIG. 5EE is superposed ³¹P-NMR spectra in the region of 56.5-53.5 ppm of SMAD7 ASO (SEQ ID NO:1) Batches P (solid line) and T (dashed line). FIG. 5FF is superposed ³¹P-NMR spectra in the region of 56.5-53.5 ppm of SMAD7 ASO (SEQ ID NO:1) Batches A, B, and D, which represent batches in three distinct areas in the PCA score plot presented in FIG. 7D.

FIG. 6 shows variable temperature circular dichroism spectra of different batches of SMAD7 ASO. Inflection points are indicated with grey circles.

FIG. 7A is superposed ³¹P-NMR spectra in the region of 57-53 ppm of SMAD7 ASO Batches A, F, G, J, K, N, Q, R, V, and Z, used as a training set in the principal component analysis (PCA). FIG. 7B is a Scree Plot generated with the ³¹P-NMR spectra of SMAD7 ASO batches used as a training set. FIG. 7C is a PCA score plot with the first two components of the ³¹P NMR spectra of the SMAD7 ASO batches used as the training set. FIG. 7D is a PCA score plot of the ³¹P-NMR spectra of the additional SMAD7 ASO batches projected onto the PCA plot generated with the training set, as displayed in FIG. 7C.

FIGS. 8A-8C show representative endoscopic images of the terminal ilea of Crohn's disease (CD) patients with no evidence of postoperative endoscopic recurrence (i0), with severe endoscopic recurrence (i4), and with established lesions (i.e., established CD) (FIG. 8A) and immunohistochemical staining of SMAD7-positive cells in CD patients at different disease stages (FIG. 8B and FIG. 8C). Insets 1-3 in FIG. 8C show higher magnification photomicrographs; inset 4 of FIG. 8C shows immunohistochemical staining with control IgG

FIGS. 9A-9C show the quantification of SMAD7-positive cells in the whole intestinal mucosa (FIG. 9A), epithelium (FIG. 9B), and lamina propria (FIG. 9C) of CD patients with no endoscopic recurrence (i0-i1), CD patients with endoscopic recurrence (i2-44), CD patients with established lesions (i.e., established CD), and control group patients. For each patient group, SMAD7-positive cells were manually counted in at least 5 high-power fields per section of 3 independent experiments. Data are presented as median and interquartile ranges.

FIGS. 10A-10C show the quantification of SMAD7-positive cells in the whole intestinal mucosa (FIG. 10A), epithelial (FIG. 10B), and lamina propria (FIG. 10C) samples from CD patients at 6 months after ileocolonic surgery, from CD patients at 12 months after ileocolonic surgery, from CD patients with established lesions at the time of ileocolonic surgery, and from control group patients. For each patient group, SMAD7-positive cells were manually counted in at least 5 high-power fields per section of 3 independent experiments. Data are presented as median and interquartile ranges.

FIG. 11A and FIG. 11B show the correlation between the number of lamina propria SMAD7-positive cells analyzed via immunohistochemistry with the percentage of IFN-γ-positive cells (FIG. 11A) and with the percentage of IL-17A-positive cells (FIG. 11B) analyzed via flow cytometry in CD patients at different stages of disease.

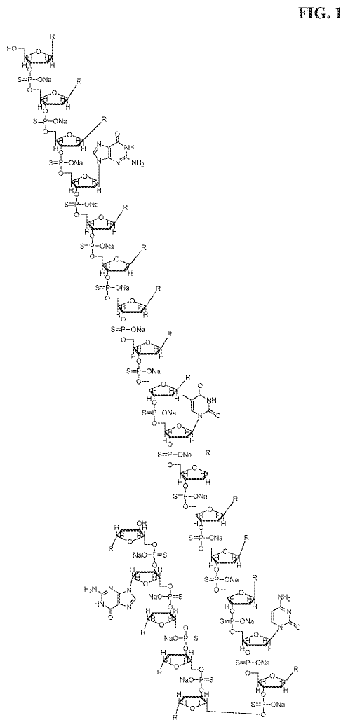

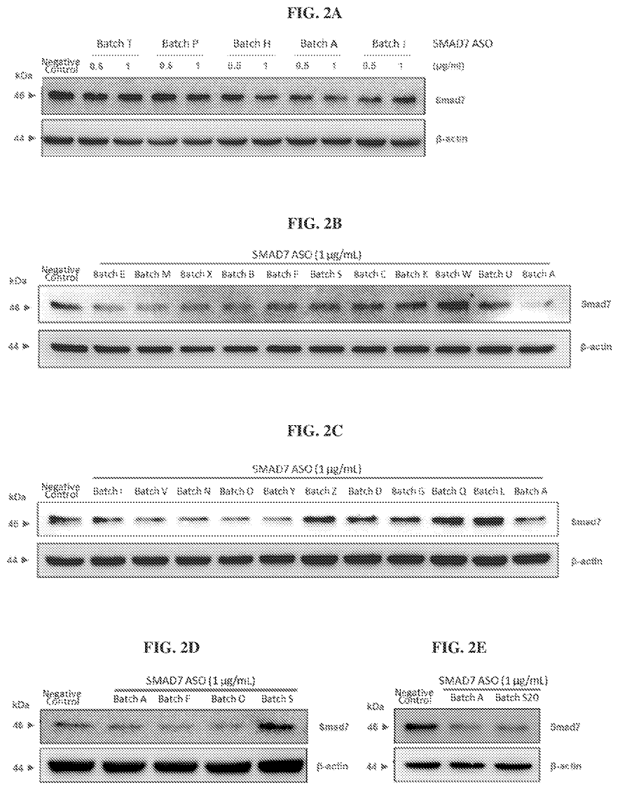

### DETAILED DESCRIPTION

Provided herein, inter alia, are compositions of an oligonucleotide, such as an antisense oligonucleotide (e.g., SMAD7 antisense oligonucleotide), comprising a plurality of diastereomers, and methods of predicting pharmacological and/or clinical efficacy, selecting, manufacturing, and using the compositions for treating, preventing, and managing a disease or disorder, such as an inflammatory bowel disease (e.g., Crohn's disease and ulcerative colitis). The methods of predicting pharmacological and/or clinical efficacy, selecting, manufacturing and using the compositions comprise obtaining, comparing, assessing, and/or characterizing spectroscopic profiles and/or performing chemometric analysis. Also provided here are methods of preventing or treating postoperative recurrence of Crohn's disease (CD) using a SMAD7 inhibitor (e.g., a SMAD7 antisense oligonucleotide). Also provided herein are methods of predicting postoperative recurrence of CD in a subject and identifying a subject at risk of postoperative recurrence of CD.

### Definitions

As used herein, “oligonucleotides” refers to short DNA or RNA molecules. Oligonucleotides can be composed of 2′-deoxyribonucleotides (oligodeoxyribonucleotides), which can be modified at the phosphate backbone or at the 2′ sugar position. In some embodiments, oligonucleotides of the disclosure have one or more phosphorothioate (PS) backbone modifications, where one or more of the non-bridging oxygen atoms in the phosphate backbone is replaced with a sulfur atom. In some embodiments, a “phosphorothioate oligonucleotide” or “PS oligonucleotide” has at least one internucleoside linkage that is an O,O-linked phosphorothioate linkage (i.e. a phosphorothioate linkage). In some embodiments, a PS oligonucleotide has all internucleoside linkages being O,O-linked phosphorothioate linkages (i.e. a phosphorothioate linkage).

“Antisense oligonucleotide,” (“ASO”) as used herein, refers to a short synthetic oligonucleotide sequence complementary to the messenger RNA (mRNA) that encodes a target protein (e.g., SMAD7). Without being bound to a particular theory, antisense oligonucleotide sequences can hybridize to a complementary region in an mRNA molecule thereby producing a double-stranded hybrid that can lead to the activation of ubiquitous catalytic enzymes, such as RNase H, which degrade DNA/RNA hybrid strands thus preventing protein translation. Without being bound by theory, an antisense oligonucleotide provided herein can hybridize to its target sequence as RNA or DNA. Thus, even if a DNA sequence is provided as a target, the corresponding RNA sequence (including uracil instead of thymine) is included as an ASO. “Phosphorothioate antisense oligonucleotides” or “PS ASOs” are antisense oligonucleotides that are modified to have a phosphorothioate backbone.

The present disclosure also provides for methods of treating inflammatory bowel disease (e.g., Crohn's disease, such as postoperative recurrence of Crohn's disease, and ulcerative colitis) via administering specific inhibitors of SMAD7. A “specific inhibitor,” as used herein, refers to an agent that has structural and/or functional properties that allow it to exclusively or with a high degree of selectivity act upon a molecular target. In some embodiments, a specific inhibitor of SMAD7 can reduce the expression and/or biological function of SMAD7 while having no, or limited effect on other molecules, e.g. other SMAD family proteins. In some embodiments, a specific inhibitor of SMAD7 may be a SMAD7 antisense oligonucleotide.

As used herein, “Mothers against decapentaplegic homolog 7” (“SMAD7,” also known as CRCS3, FLJ16482, MADH7, MADH8, MAD (mothers against decapentaplegic, Drosophila) homolog 7, MAD homolog 8. SMAD, mothers against DPP homolog 7, mothers against DPP homolog 8) means the human protein or any of the mRNA transcripts encoded by the gene identified by Entrez GeneID No. 4092 and allelic variants thereof.

As used herein, “SMAD7 antisense oligonucleotide” or “SMAD7 ASO” is understood to refer to an oligonucleotide comprising a nucleic acid sequence that is complementary to a nucleic acid sequence in an mRNA molecule transcribed from the SMAD7 gene. More specifically, such an oligonucleotide can be complementary to the nucleic acid sequence in the coding region of such an mRNA. In some embodiments, a SMAD7 antisense oligonucleotide possesses the inherent functional property of targeting the SMAD7 gene, its RNA or protein products, or another molecular entity whose activity or expression impinges upon the activity or expression of SMAD7 or its products either exclusively or with a high degree of specificity. In some embodiments, a SMAD7 antisense oligonucleotide can reduce the expression of SMAD7 when introduced into a cell (e.g., an immune cell, such as a PBMC, dendritic cell, or B-cell). In some embodiments, a SMAD7 antisense oligonucleotide can reduce expression of an mRNA transcribed from the gene. In some embodiments, a SMAD7 antisense oligonucleotide can reduce expression of a protein encoded by the gene. In some embodiments, a SMAD7 antisense oligonucleotide can reduce secretion of a protein encoded by the gene from the cell into which the SMAD7 antisense oligonucleotide was introduced. In some embodiments, a SMAD7 antisense oligonucleotide comprises the sequence of SEQ ID NOs: 1, 2, 3, 4, 5, or 6, or pharmaceutically acceptable salts thereof.

As used herein, “pharmacological efficacy” refers to the ability of a drug substance to produce a desired or intended therapeutic effect. A “pharmacologically efficacious” drug substance, therefore, is capable of producing a desired or intended therapeutic effect. As used herein, “clinical efficacy” refers to the ability of a drug substance to produce a desired or intending effect when administered to a subject. “Therapeutic effect” refers to the response(s) after a treatment of any kind, the results of which are judged to be useful or favorable. It is to be understood that the establishment of the efficacy of a drug substance is often done relative to other available drug substances, with which it will be compared. In some embodiments, pharmacological and/or clinical efficacy of a drug substance may be evaluated by means of presence, absence or degree of one or more symptoms associated with a disease or disorder (e.g., Crohn's disease and ulcerative colitis), analysis of tissue histology, biochemical assay, imaging methods such as magnetic resonance imaging, or other known methods. For instance, pharmacological and/or clinical efficacy of a SMAD7 antisense oligonucleotide may be evaluated by Crohn's Disease Activity Index (CDAI) scoring following administration of a SMAD7 antisense oligonucleotide to a Crohn's Disease patient. Biochemical assays that examine protein or RNA expression may also be used to evaluate pharmacological and/or clinical efficacy. One may also evaluate the presence or level of expression of useful biomarkers to evaluate the progression of the disease or disorder and thereby the pharmacological and/or clinical efficacy.

The terms “patient.” “individual,” and “subject” are used interchangeable and refer to any animal suffering from or diagnosed with a disease or disorder, such as an inflammatory bowel disease, e.g. Crohn's disease (such as postoperative recurrence of Crohn's disease) or ulcerative colitis, including, but not limited to, mammals, primates, and humans. In certain embodiments, the patient may be a non-human mammal such as, for example, a cat, a dog, or a horse. In a preferred embodiment, the patient is a human subject. None of the terms require the supervision of a medical professional.

The terms “disease,” “disorder,” and “condition” are used interchangeably herein.

“Treating,” includes any effect, e.g., lessening, reducing, modulating, preventing, or eliminating, that results in the improvement of a condition, disease, disorder, symptom, etc. “Treating” or “treatment” of a disease state includes: (1) inhibiting the disease state, i.e., arresting the development of the disease state or its clinical symptoms; (2) relieving the disease state. i.e., causing temporary or permanent regression of the disease state or its clinical symptoms; (3) reducing or lessening the symptoms of the disease state; or (4) preventing the disease state, e.g., causing the clinical symptoms of the disease state not to develop in a subject that may be exposed to or predisposed to the disease state, but does not yet experience or display symptoms of the disease state.

As used herein, “preventing” or “prevent” describe reducing or eliminating the onset of the symptoms or complications of the disease, condition or disorder. The term “preventing,” when used in relation to a condition, such as inflammation or the recurrence of Crohn's disease, is art-recognized, and refers to the ability of a formulation, composition, and/or device to reduce the frequency of, or delay the onset of, signs and/or symptoms of a medical condition in a subject relative to a subject which does not receive the formulation, composition, and/or device. Insofar as the methods of the present disclosure are directed to preventing disorders, it is understood that the term “prevent” does not require that the disease state be completely thwarted.

As used herein, “reduce,” or other forms of the word, such as “reducing” or “reduction,” refers to decreasing the incidence of an event or lessening a characteristic (e.g., inflammation or lesion). It is understood that this is typically in relation to some standard or expected value, in other words it is relative, but that it is not always necessary for the standard or relative value to be referred to.

As used herein, “ameliorating a symptom.” or other forms of the term such as “ameliorate a symptom,” refers to the mitigation of one or more symptoms of a disease or a disorder in a host and/or reduces, inhibits, or eliminates a particular symptom associated with the disease or disorder prior to and/or post-administration of a therapeutic agent.

As used herein, “manage,” “management,” “managing,” and the like generally refers to controlling the severity or manifestation of symptoms of a disease, or the means of treating the disease. Generally, management is used to obtain a desired pharmacological, physiological, and/or clinical effect. The effect may be therapeutic in terms of partially or completely curing a disease and/or adverse effect attributed to the disease or ensuring that a particular symptom or manifestation of the disease does not occur or reoccur in a patient or does not rise to an undesirable or intolerable level in a patient. The term “management” as used herein covers any management of a disease in a mammal, particularly a human, and includes: (a) inhibiting the disease, i.e. preventing the disease from increasing in severity or scope; (b) relieving the disease, i.e. causing partial or complete amelioration of the disease; or (c) preventing relapse of the disease. i.e. preventing the disease from returning to an active state following previous successful treatment of symptoms of the disease or treatment of the disease.

The term “Crohn's disease” (“CD”), as used herein, shall be given its ordinary meaning, as an inflammatory bowel disease that causes chronic inflammation of the gastrointestinal tract. Crohn's disease can affect any part of the gastrointestinal tract, but most often affects the small intestine and colon. Surgical intervention is often required in Crohn's disease patients.

Ins some embodiments, Crohn's disease as used herein may be postoperative recurrence of Crohn's disease. Postoperative recurrence of Crohn's disease may manifest by histologic or endoscopic findings or with clinical symptoms. “Histologic recurrence (HR)” is the presence of histological activity on mucosal biopsies obtained during ileocolonoscopy, which may be observed within one week of the surgery. Endoscopic evaluation is a valuable tool in determining postoperative recurrence and guiding medical management. The Rutgeert's scoring system is most widely used to grade “endoscopic recurrence (ER)”. The score is assessed by evaluating the ileum proximal to the anastomosis (neoterminal ileum): i0 indicates no evidence of lesions; i1, 5 or less aphthous lesions; i2 applies to more than 5 aphthous lesions with normal mucosa between the lesions, or skip areas of larger lesions, or lesions confined to the ileocolonic anastomotic lining; i3 indicates diffuse aphthous ileitis with diffusely inflamed mucosa; and i4 presents diffuse ileal inflammation with larger ulcers, nodules, or narrowing. “Radiographic recurrence” is the exhibition of symptoms observed with radiographic methods, such as contrast ultra-sonography and computed tomography (CT) or magnetic resonance (MR) enteroclysis. “Clinical recurrence” is defined using the Crohn's Disease Activity Index (CDAI), a tool used to quantify the symptoms of patients with Crohn's disease, as, e.g., CDAI>150 or CDAI>200.

Subjects in need of treatment by the methods described herein include subjects having Crohn's disease, such as postoperative recurrence of Crohn's disease. In some embodiments, the subject may have endoscopic recurrence of Crohn's disease. Signs of endoscopic recurrence include, but are not limited to, lesions (e.g., mucosal lesions), aphthae, ulcers, narrowing of the lumen in the neoterminal ileum, abscesses, fistulas, joint pain, diarrhea, stomach pain or cramping, fever, bloody stool, and anemia.

Throughout the description and claims of this specification the word “comprise” and other forms of the word, such as “comprising” and “comprises,” means including but not limited to, and is not intended to exclude, for example, other additives, components, integers, or steps.

“Optional” or “optionally” means that the subsequently described event or circumstance can or cannot occur, and that the description includes instances where the event or circumstance occurs and instances where it does not.

As used herein, the term “about” or “approximately,” when used in reference to a quantitative value, includes the recited quantitative value itself, unless specifically stated otherwise. As used herein, the term “about” or “approximately” refers to a ±10% variation from the recited quantitative value unless otherwise indicated or inferred from the context.

The terminology used herein is for the purpose of describing particular embodiments only, and is not intended to limit the scope of the present disclosure. As used throughout this disclosure, the singular forms “a,” “an,” and “the” include plural reference unless the context clearly dictates otherwise. Thus, for example, a reference to “a composition” includes a plurality of such compositions, as well as a single composition, and a reference to “a therapeutic agent” is a reference to one or more therapeutic and/or pharmaceutical agents and equivalents thereof known to those skilled in the art, and so forth. All percentages and ratios used herein, unless otherwise indicated, are by weight.

Unless otherwise defined, all technical and scientific terms used herein have the same meaning as commonly understood by one of ordinary skill in the art to which this disclosure belongs. In the case of conflict, the present specification will control. Although methods and materials similar or equivalent to those described herein can be used in the practice or testing of the present disclosure, suitable methods and materials are described below.

### Oligonucleotides of the Disclosure

Provided herein, inter alia, are pharmaceutical compositions comprising a plurality of diastereomers of a SMAD7 antisense oligonucleotide (ASO), wherein the plurality of diastereomers of a SMAD7 ASO has a phosphorus-31 nuclear magnetic resonance (31P-NMR) spectrum comprising: (a) one or more resonances between 54.8 to 55.5 ppm; and (b) a first principal component 1 (PC1) score and a second principal component 2 (PC2) score, wherein (i) the PC1 score is below about −0.20 or above about 0.25 and the PC2 score is below about 0.00; or (ii) the PC1 score is outside of the range of −0.32 to 0.31 and/or the PC2 score that is outside the range of 0.00 to 0.20.

In some embodiments, the SMAD7 ASO has a sequence at least 90% identical to SEQ ID NO: 1 (5′-GTXGCCCCTTCTCCCXGCAGC-3′), in which X is 5-methyl 2′-deoxycytidine. In some embodiments, the pharmaceutical compositions comprise a plurality of diastereomers of a SMAD7 ASO having a sequence at least 95% identical to SEQ ID NO: 1 (5′-GTXGCCCCTTCTCCCXGCAGC-3′), in which X is 5-methyl 2′-deoxycytidine. In some embodiments, the pharmaceutical compositions comprise a plurality of diastereomers of a SMAD7 ASO having a sequence according to SEQ ID NO: 1 (5′-GTXGCCCCTTCTCCCXGCAGC-3′), in which X is 5-methyl 2′-deoxycytidine.

In some embodiments, the SMAD7 ASO has a sequence at least 90% identical to SEQ ID NO: 2 (5′-GTXGCCCCTTCTCTCXGCAGC-3′) in which X is 5-methyl 2′-deoxycytidine. In some embodiments, the pharmaceutical compositions comprise a plurality of diastereomers of a SMAD7 ASO having a sequence at least 95% identical to SEQ ID NO: 2. In some embodiments, the pharmaceutical compositions comprise a plurality of diastereomers of a SMAD7 ASO having a sequence according to SEQ ID NO: 2.

In some embodiments, the SMAD7 ASO has a sequence at least 90% identical to SEQ ID NO: 3 (5′-GTXYCCCCTTCTCCCXYCAG-3′), in which X is a nucleotide including a nitrogenous base of cytosine, 5-methylcytosine, or a 2′-O-methylcytosine and Y is a nucleotide including a nitrogenous base of guanine, 5-methylguanine, or a 2′-O-methylguanine, provided that at least one of X and Y includes a methylated nitrogenous base. In some embodiments, the pharmaceutical compositions comprise a plurality of diastereomers of a SMAD7 ASO having a sequence at least 95% identical to SEQ ID NO: 3. In some embodiments, the pharmaceutical compositions comprise a plurality of diastereomers of a SMAD7 ASO having a sequence according to SEQ ID NO: 3.

In some embodiments, the SMAD7 ASO has a sequence at least 90% identical to SEQ ID NO: 4 (5′-GTXGCCCCTTCTCCCXGCAG-3′), in which X is 5-methyl 2′-deoxycytidine. In some embodiments, the pharmaceutical compositions comprise a plurality of diastereomers of a SMAD7 ASO having a sequence at least 95% identical to SEQ ID NO: 4. In some embodiments, the pharmaceutical compositions comprise a plurality of diastereomers of a SMAD7 ASO having a sequence according to SEQ ID NO: 4.

In some embodiments, the SMAD7 ASO has a sequence at least 90% identical to SEQ ID NO: 5 (5′-GTC*GCCCCTTCTCCCC*YCAGC-3′), in which C* is 5-methyl-2′-deoxycytidine and Y is a nucleotide including a nitrogenous base of guanine, 5-methylguanine, or a 2′-O-methylguanine. In some embodiments, the pharmaceutical compositions comprise a plurality of diastereomers of a SMAD7 ASO having a sequence at least 95% identical to SEQ ID NO: 5. In some embodiments, the pharmaceutical compositions comprise a plurality of diastereomers of a SMAD7 ASO having a sequence according to SEQ ID NO: 5.

In some embodiments, the SMAD7 ASO has a sequence at least 90% identical to SEQ ID NO: 6 (5′-GTC*GCCCCTTCTCTCC*YCAGC-3′), in which C* is 5-methyl-2′-deoxycytidine and Y is a nucleotide including a nitrogenous base of guanine, 5-methylguanine, or a 2′-O-methylguanine. In some embodiments, the pharmaceutical compositions comprise a plurality of diastereomers of a SMAD7 ASO having a sequence at least 95% identical to SEQ ID NO: 6. In some embodiments, the pharmaceutical compositions comprise a plurality of diastereomers of a SMAD7 ASO having a sequence according to SEQ ID NO: 6.

In some embodiments, the ASO is chemically modified. In some embodiments, the ASO is a phosphorothioate (PS) oligonucleotide wherein one of the non-bridging oxygen atoms in the phosphate backbone is replaced with a sulfur atom. In some embodiments, the ASO has at least one internucleoside linkage that is an O,O-linked phosphorothioate linkage (i.e., a phosphorothioate linkage). In some embodiments, the ASO has all internucleoside linkages being O,O-linked phosphorothioate linkages (i.e., a phosphorothioate linkage).

In some embodiments, contemplated compositions disclosed herein may include a pharmaceutically acceptable salt, e.g., a sodium salt of the antisense oligonucleotide of SEQ ID NO: 1, SEQ ID NO: 2, SEQ ID NO: 3, SEQ ID NO: 4, SEQ ID NO: 5, or SEQ ID NO: 6, that optionally may include 1 to 20 O,O-linked phosphorothioate internucleoside linkages (i.e. phosphorothioate bonds). In some embodiments, the contemplated antisense oligonucleotide is an antisense oligonucleotide comprising the free acid form, the salt form, or the anionic form without a counterion of SEQ ID NO: 1, SEQ ID NO: 2, SEQ ID NO: 3, SEQ ID NO: 4, SEQ ID NO: 5, or SEQ ID NO: 6, wherein each of the 20 internucleoside linkages is an O,O-linked phosphorothioate linkage. In some embodiments, the phosphorothioate backbone of SEQ ID NO: 1, SEQ ID NO: 2, SEQ ID NO: 3, SEQ ID NO: 4, SEQ ID NO: 5, or SEQ ID NO: 6, can be fully or partially protonated to form an acidic form of SEQ ID NO: 1, SEQ ID NO: 2, SEQ ID NO: 3. SEQ ID NO: 4, SEQ ID NO: 5, or SEQ ID NO: 6. Contemplated salts of oligonucleotides include those that are fully neutralized, e.g., each phosphorothioate linkage is associated with an ion such as Na⁺. In some embodiments, the salt of the antisense oligonucleotide of SEQ ID NO: 1, SEQ ID NO: 2. SEQ ID NO: 3, SEQ ID NO: 4, SEQ ID NO: 5, or SEQ ID NO: 6 is only partially neutralized, e.g., less than all phosphorothioate linkages are associated with an ion (e.g., less than 99%, less than 95%, less than 90%, less than 85%, less than 80%, less than 75%, less than 70%, less than 65%, less than 60%, less than 55%, less than 50%, less than 45%, less than 40%, less than 35%, less than 30%, less than 25%, less than 20%, less than 15%, less than 10%, less than 5%, less than 3%, or less than 1% are neutralized). Oligonucleotides may include naturally occurring nucleobases, sugars, and covalent internucleoside (backbone) linkages as well as non-naturally occurring portions. In varying embodiments, the antisense oligonucleotides of the present disclosure, for example, the antisense oligonucleotide of SEQ ID NO: 1, SEQ ID NO: 2, SEQ ID NO: 3. SEQ ID NO: 4, SEQ ID NO: 5, or SEQ ID NO: 6, include or may include nucleotides including deoxycytidine and/or 5-methyl 2′-deoxycytidine, including, but not limited to, 5-methyl-2′-deoxycytidine 5′-monophosphate and 5-methyl-2′-deoxycytidine 5′-monophosphorothioate.

In some embodiments, the pharmaceutical compositions comprising a plurality of diastereomers of SEQ ID NO: 1 comprise greater than 50 millimoles, greater than 100 millimoles, greater than 200 millimoles, greater than 300 millimoles, greater than 400 millimoles, greater than 500 millimoles, greater than 600 millimoles, greater than 700 millimoles, greater than 800 millimoles, greater than 900 millimoles, greater than 1 mole, greater than 1.5 moles, greater than 2 moles, greater than 2.5 moles, greater than 3 moles, greater than 4 moles, or greater than 5 moles of the plurality of diastereomers of SEQ ID NO: 1. In some embodiments, the pharmaceutical compositions comprising a plurality of diastereomers of SEQ ID NO: 1 comprise greater than 2 moles of the plurality of diastereomers of SEQ ID NO: 1. In some embodiments, the pharmaceutical composition comprises less than 5 moles, less than 4 moles, less than 3 moles, less than 2.5 moles, less than 2 moles, less than 1.5 moles, less than 1 moles, less than 900 millimoles, less than 850 millimoles, less than 800 millimoles, less than 750 millimoles, less than 700 millimoles, less than 650 millimoles, less than 600 millimoles, less than 550 millimoles, less than 500 millimoles, less than 450 millimoles, less than 400 millimoles, less than 350 millimoles, less than 300 millimoles, less than 250 millimoles, less than 200 millimoles, less than 150 millimoles, or less than 100 millimoles of the plurality of diastereomers of SEQ ID NO: 1. In some embodiments, the pharmaceutical composition comprises greater than 100 millimoles to about 5 moles of the plurality of diastereomers of SEQ ID NO: 1. In some embodiments, the pharmaceutical composition comprises greater than 100 millimoles to about 2 moles of the plurality of diastereomers of SEQ ID NO: 1. In some embodiments, the pharmaceutical composition comprises greater than 300 millimoles to about 5 moles of the plurality of diastereomers of SEQ ID NO: 1. In some embodiments, the pharmaceutical composition comprises greater than 300 millimoles to about 2 moles of the plurality of diastereomers of SEQ ID NO: 1. In some embodiments, the pharmaceutical composition comprises greater than 900 millimoles to about 5 moles of the plurality of diastereomers of SEQ ID NO: 1. In some embodiments, the pharmaceutical composition comprises greater than 900 millimoles to about 2 moles of the plurality of diastereomers of SEQ ID NO: 1. In some embodiments, the pharmaceutical composition comprises greater than 300 millimoles to about 900 millimoles of the plurality of diastereomers of SEQ ID NO: 1. In some embodiments, the pharmaceutical composition comprises greater than 300 millimoles to about 5 moles, greater than 300 millimoles to about 4 moles, greater than 300 millimoles to about 3 moles, greater than 300 millimoles to about 2.5 moles, greater than 300 millimoles to about 2 moles, greater than 300 millimoles to about 1.5 moles, greater than 300 millimoles to about 1 mole, greater than 300 millimoles to about 900 millimoles, greater than 300 millimoles to about 800 millimoles, greater than 300 millimoles to about 700 millimoles, greater than 300 millimoles to about 600 millimoles, greater than 300 millimoles to about 500 millimoles, greater than 300 millimoles to about 400 millimoles, greater than 400 millimoles to about 5 moles, greater than 400 millimoles to about 4 moles, greater than 400 millimoles to about 3 moles, greater than 400 millimoles to about 2.5 moles, greater than 400 millimoles to about 2 moles, greater than 400 millimoles to about 1.5 moles, greater than 400 millimoles to about 1 mole, greater than 400 millimoles to about 900 millimoles, greater than 400 millimoles to about 800 millimoles, greater than 400 millimoles to about 700 millimoles, greater than 400 millimoles to about 600 millimoles, greater than 400 millimoles to about 500 millimoles, greater than 500 millimoles to about 5 moles, greater than 500 millimoles to about 4 moles, greater than 500 millimoles to about 3 moles, greater than 500 millimoles to about 2.5 moles, greater than 500 millimoles to about 2 moles, greater than 500 millimoles to about 1.5 moles, greater than 500 millimoles to about 1 mole, greater than 500 millimoles to about 900 millimoles, greater than 500 millimoles to about 800 millimoles, greater than 500 millimoles to about 700 millimoles, greater than 500 millimoles to about 600 millimoles, greater than 600 millimoles to about 5 moles, greater than 600 millimoles to about 4 moles, greater than 600 millimoles to about 3 moles, greater than 600 millimoles to about 2.5 moles, greater than 600 millimoles to about 2 moles, greater than 600 millimoles to about 1.5 moles, greater than 600 millimoles to about 1 mole, greater than 600 millimoles to about 900 millimoles, greater than 600 millimoles to about 800 millimoles, greater than 600 millimoles to about 700 millimoles, greater than 700 millimoles to about 5 moles, greater than 700 millimoles to about 4 moles, greater than 700 millimoles to about 3 moles, greater than 700 millimoles to about 2.5 moles, greater than 700 millimoles to about 2 moles, greater than 700 millimoles to about 1.5 moles, greater than 700 millimoles to about 1 mole, greater than 700 millimoles to about 900 millimoles, greater than 700 millimoles to about 800 millimoles, greater than 800 millimoles to about 5 moles, greater than 800 millimoles to about 4 moles, greater than 800 millimoles to about 3 moles, greater than 800 millimoles to about 2.5 moles, greater than 800 millimoles to about 2 moles, greater than 800 millimoles to about 1.5 moles, greater than 800 millimoles to about 1 mole, greater than 800 millimoles to about 900 millimoles, greater than 900 millimoles to about 5 moles, greater than 900 millimoles to about 4 moles, greater than 900 millimoles to about 3 moles, greater than 900 millimoles to about 2.5 moles, greater than 900 millimoles to about 2 moles, greater than 900 millimoles to about 1.5 moles, greater than 900 millimoles to about 1 mole, greater than 1 mole to about 5 moles, greater than 1 mole to about 4 moles, greater than 1 mole to about 3 moles, greater than 1 mole to about 2.5 moles, greater than 1 mole to about 2 moles, greater than 1 mole to about 1.5 moles, greater than 1.5 moles to about 5 moles, greater than 1.5 moles to about 4 moles, greater than 1.5 moles to about 3 moles, greater than 1.5 moles to about 2.5 moles, greater than 1.5 moles to about 2 moles, greater than 2 moles to about 5 moles, greater than 2 moles to about 4 moles, greater than 2 moles to about 3 moles, greater than 2 moles to about 2.5 moles, greater than 2.5 moles to about 5 moles, greater than 2.5 moles to about 4 moles, greater than 2.5 moles to about 3 moles, greater than 3 moles to about 5 moles, greater than 3 moles to about 4 moles, or greater than 4 moles to about 5 moles of the plurality of diastereomers of SEQ ID NO: 1. In some embodiments, the pharmaceutical composition comprises about 1 moles, about 900 millimoles, about 850 millimoles, about 800 millimoles, about 750 millimoles, about 700 millimoles, about 650 millimoles, about 600 millimoles, about 550 millimoles, about 500 millimoles, about 450 millimoles, about 400 millimoles, about 350 millimoles, about 300 millimoles, about 250 millimoles, about 200 millimoles, about 150 millimoles, or about 100 millimoles of the plurality of diastereomers of SEQ ID NO: 1.

In some embodiments, an oligonucleotide targeting SMAD7 of the disclosure (e.g., SEQ ID NO: 1) or a pharmaceutical composition disclosed herein (e.g., a composition comprising a plurality of diastereomers of SEQ ID NO: 1) down-regulates SMAD7 expression. In some embodiments, the SMAD7 expression is SMAD7 protein and/or mRNA expression. In some embodiments, the SMAD7 protein expression is down-regulated in a cell by more than about 10%, more than about 15%, more than about 20%, more than about 25%, more than about 30%, more than about 35%, more than about 40%, more than about 45%, more than about 50%, more than about 55%, more than about 60%, or more than about 65% compared to an untreated cell. In some embodiments, the SMAD7 protein expression is down-regulated in a cell by more than about 40% compared to an untreated cell. In some embodiments, the down-regulation of SMAD7 protein expression in a cell may be determined as described in Example 2.

### Diastereomer Profile

In some embodiments, the plurality of diastereomers has a characterized diastereomeric profile. In some embodiments, the plurality of diastereomers has a spectroscopic profile indicative of pharmacological and/or clinical efficacy. The spectroscopic profile indicative of pharmacological and/or clinical efficacy is at least one of a ³¹P-NMR spectrum, a ¹H-NMR spectrum, a ¹³C-NMR spectrum, and a VTCD spectrum.

In some embodiments, the spectroscopic profile indicative of pharmacological and/or clinical efficacy is a ³¹P-NMR spectrum. In some embodiments, the ³¹P-NMR is performed at about 14.1 T. In some embodiments, the ³¹P-NMR is performed in solution.

In some embodiments, the ³¹P-NMR spectrum has one or more resonances between 54.8 to 55.8 ppm. In some embodiments, the ³¹P-NMR spectrum has one or more resonances between 54.8 to 55.5 ppm. In some embodiments, the ³¹P-NMR spectrum has two or more resonances between 54.8 to 55.5 ppm. In some embodiments, the ³¹P-NMR spectrum has two or more resonances between 54.8 to 55.5 ppm with different intensities. In some embodiments, the ³¹P-NMR spectrum has one or more resonances between 55.0 to 55.4 ppm. In some embodiments, the ³¹P-NMR spectrum has, independently at 54.8 ppm, 54.9 ppm, 55.0 ppm, 55.1 ppm, 55.2 ppm, 55.3 ppm, 55.4 ppm, and/or 55.5 ppm, one or more resonances. In some embodiments, the ³¹P-NMR spectrum has one or more resonances at about 54.8 ppm. In some embodiments, the ³¹P-NMR spectrum has one or more resonances at about 54.9 ppm. In some embodiments, the ³¹P-NMR spectrum has one or more resonances at about 55.0 ppm. In some embodiments, the ³¹P-NMR spectrum has one or more resonances at about 55.1 ppm. In some embodiments, the ³¹P-NMR spectrum has one or more resonances at about 55.2 ppm. In some embodiments, the ³¹P-NMR spectrum has one or more resonances at about 55.3 ppm. In some embodiments, the ³¹P-NMR spectrum has one or more resonances at about 55.4 ppm. In some embodiments, the ³¹P-NMR spectrum has one or more resonances at about 55.5 ppm. In some embodiments, the ³¹P-NMR spectrum has, independently at 54.8 ppm, 54.9 ppm, 55.0 ppm, 55.1 ppm, 55.2 ppm, 55.3 ppm, 55.4 ppm, and/or 55.5 ppm, two or more resonances. In some embodiments, the ³¹P-NMR spectrum has two or more resonances at about 54.8 ppm. In some embodiments, the ³¹P-NMR spectrum has two or more resonances at about 54.9 ppm. In some embodiments, the ³¹P-NMR spectrum has two or more resonances at about 55.0 ppm. In some embodiments, the ³¹P-NMR spectrum has two or more resonances at about 55.1 ppm. In some embodiments, the ³¹P-NMR spectrum has two or more resonances at about 55.2 ppm. In some embodiments, the ³¹P-NMR spectrum has two or more resonances at about 55.3 ppm. In some embodiments, the ³¹P-NMR spectrum has two or more resonances at about 55.4 ppm. In some embodiments, the ³¹P-NMR spectrum has two or more resonances at about 55.5 ppm.

In some embodiments, the ³¹P-NMR spectrum, when subjected to principal component analysis (PCA), comprises a first principal component 1 (PC1) score that is below about −0.20 or above about 0.25 and a second principal component 2 (PC2) score that is below about 0.00. In some embodiments, the ³¹P-NMR spectrum, when subjected to PCA, comprises a PC1 score is outside of the range of −0.32 to 0.31 and/or a PC2 score that is outside the range of 0.00 to 0.20. In some embodiments, the ³¹P-NMR spectrum, when subjected to PCA, comprises a PC1 score that is below about −0.20 and a PC2 score that is below about 0.00. In some embodiments, the ³¹P-NMR spectrum, when subjected to PCA, comprises a PC1 score that is above about 0.25 and a PC2 score that is below about 0.00. In some embodiments, the ³¹P-NMR spectrum, when subjected to PCA, comprises a PC1 score that is from about −0.47 to about −0.20 and a PC2 score that is from about −0.27 to about 0.00. In some embodiments, the ³¹P-NMR spectrum, when subjected to PCA, comprises a PC1 score that is from about 0.65 to about 0.9 and a PC2 score that is from about −0.47 to about 0.00. In some embodiments, the ³¹P-NMR spectrum, when subjected to PCA, comprises a PC1 score that is outside of the range of −0.32 to 0.30 when the PC2 score is above 0.00 and below 0.20. In some embodiments, the ³¹P-NMR spectrum, when subjected to PCA, comprises a PC2 score is outside the range of 0.00 to 0.20 when the PC1 is above −0.32 and below 0.30.

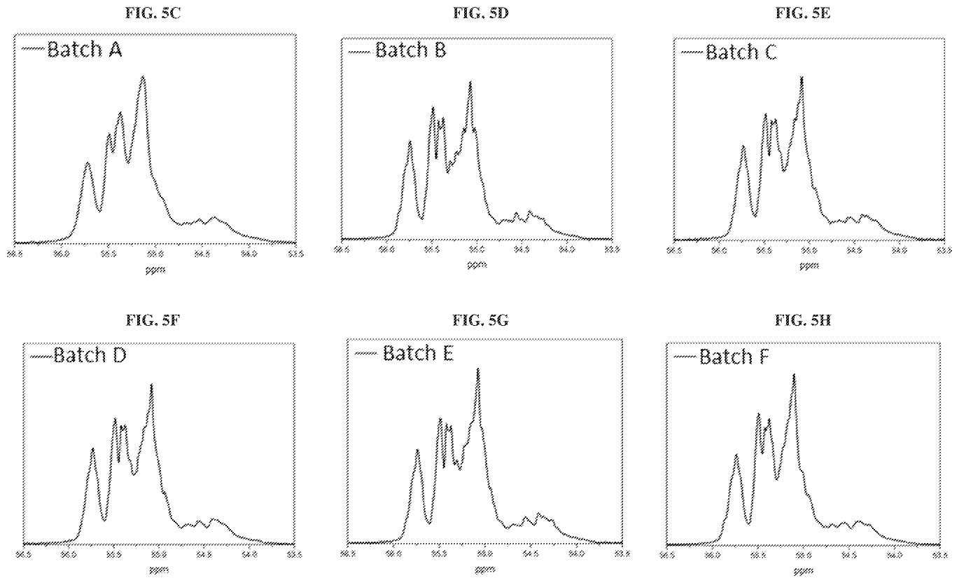

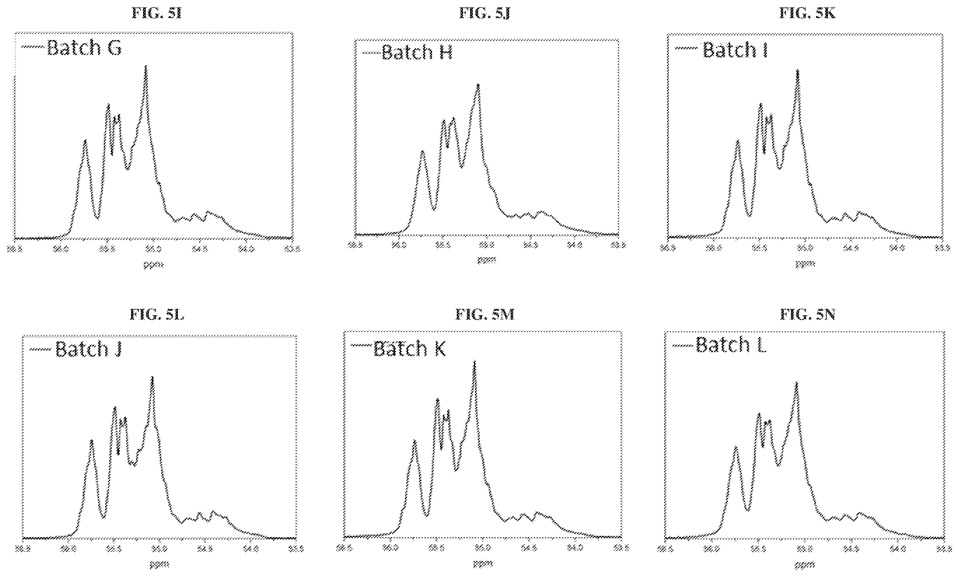

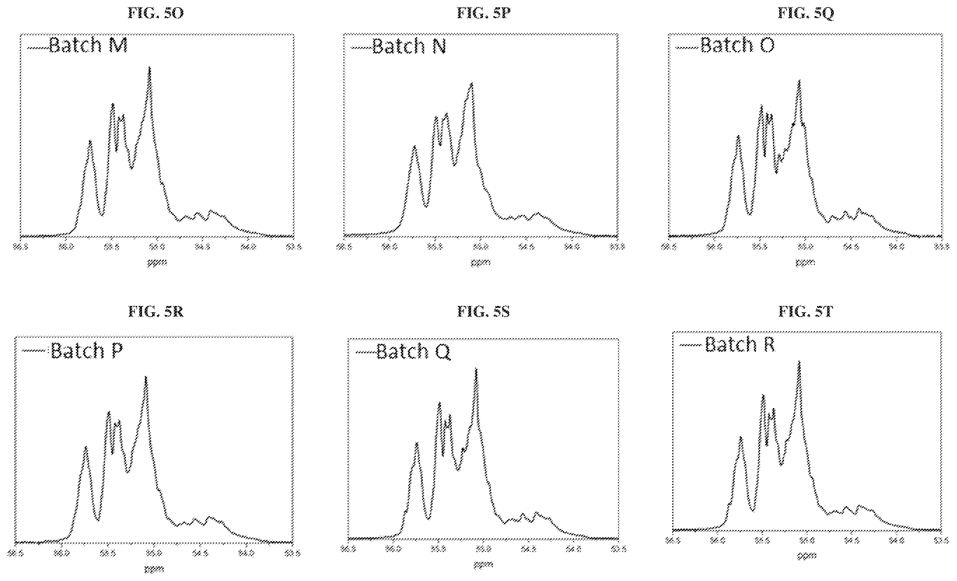

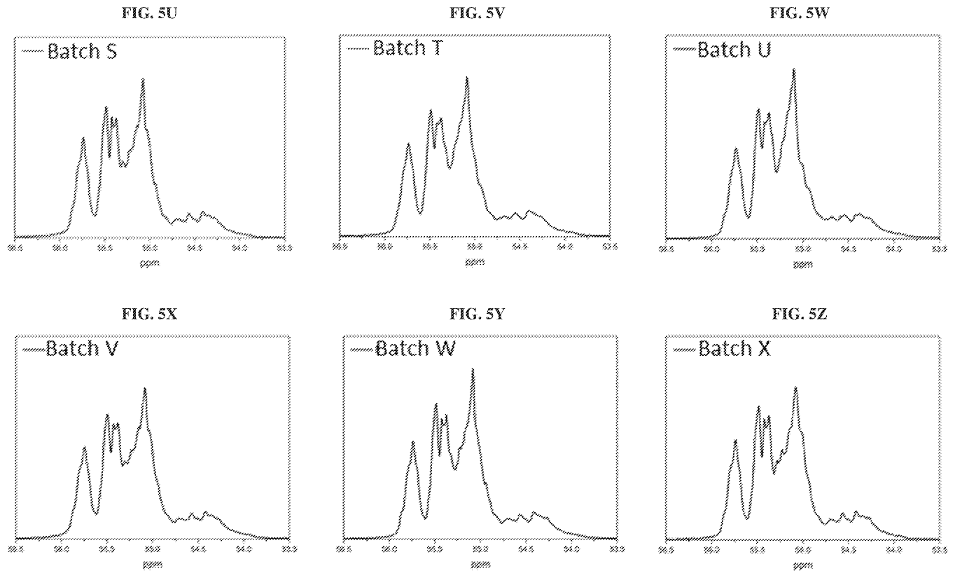

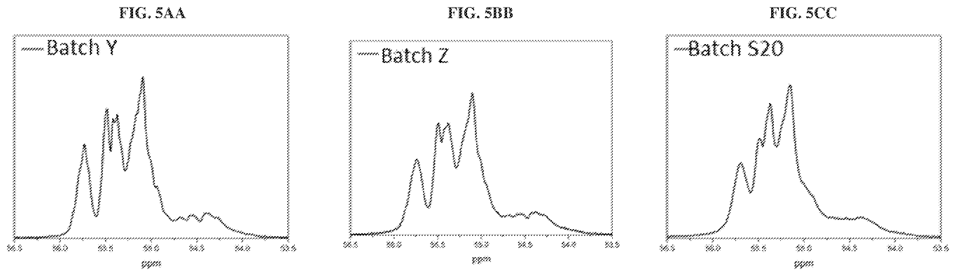

In some embodiments, the PCA comprises selecting principle components using a modeling data set. In some embodiments, the modeling data set comprises five, six, seven, eight, nine, ten, or up to twenty-seven ³¹P-NMR spectra, e.g., selected from the ³¹P-NMR spectra shown in FIGS. 5C-5CC. In some embodiments, the modeling data comprises ten ³¹P-NMR spectra, e.g., the ten ³¹P-NMR spectra shown in FIGS. 5C, 5L, 5X, 5H, 5P, 5I, 5M, 5T, 5S, and 5BB.

In some embodiments, the principal components are selected to account for more than 65%, more than 70%, more than 75%, more than 80%, more than 85%, more than 90%, more than 95%, or more than 99% variance of the modeling data set. In some embodiments, the principal components are selected to account for more than 90% variance of the modeling data set. In some embodiments, the PCA may be performed as described in Example 7.

In some embodiments, the spectroscopic profile indicative of pharmacological and/or clinical efficacy is a ¹H-NMR spectrum. In some embodiments, the spectroscopic profile indicative of pharmacological and/or clinical efficacy is a ¹³C-NMR spectrum.

In some embodiments, the spectroscopic profile indicative of pharmacological and/or clinical efficacy is at least a VTCD spectrum. In some embodiments, the VTCD spectrum has at least one inflection point between 40° C. to 55° C. In some embodiments, the VTCD spectrum has at least one inflection point between 42° C. to 48° C. In some embodiments, the VTCD spectrum has at least one inflection point between 44° C. to 46° C. In some embodiments, the VTCD spectrum has an inflection point at about 45° C.

### Methods of Treating an Inflammatory Bowel Disease of the Disclosure

Provided herein, in certain embodiments, are pharmaceutical compositions for use in treating an inflammatory bowel disease, comprising a plurality of diastereomers of a SMAD7 antisense oligonucleotide (e.g., an antisense oligonucleotide of SEQ ID NO: 1, 2, 3, 4, 5, or 6, or a pharmaceutically acceptable salt thereof), wherein the plurality of diastereomers has a ³¹P-NMR spectrum comprising: a) one or more resonances between 54.8 to 55.5 ppm; and b) (i) a first principal component 1 (PC1) score that is below about −0.20 or above about 0.25 and a second principal component 2 (PC2) score that is below about 0.00; or (ii) a PC1 score is outside of the range of −0.32 to 0.31 and/or a PC2 score that is outside the range of 0.00 to 0.20. In some embodiments, the SMAD7 antisense oligonucleotide comprises a sequence selected from any one of SEQ ID NOs: 1-6 or a pharmaceutically acceptable salt thereof. In some embodiments, the SMAD7 antisense oligonucleotide has a sequence according to SEQ ID NO: 1 (5′-GTXGCCCCTTCTCCCXGCAGC-3′) in which all internucleoside linkages are O,O-linked phosphorothioate linkages and X is 5-methyl 2′-deoxycytidine.

The plurality of diastereomers of a SMAD7 antisense oligonucleotide of the provided method has a spectroscopic profile indicative of pharmacological and/or clinical efficacy. In some embodiments, the spectroscopic profile indicative of pharmacological and/or clinical efficacy is a ³¹P-NMR spectrum comprising: a) one or more resonances between 54.8 to 55.5 ppm; and b) a first principal component 1 (PC1) score and a second principal component 2 (PC2) score as determined by principal component analysis (PCA), wherein (i) the PC1 score is below about −0.20 or above about 0.25 and the PC2 score is below about 0.00, or (ii) the PC1 score is outside of the range of −0.32 to 0.31 and/or the PC2 score that is outside the range of 0.00 to 0.20. In some embodiments, the ³¹P-NMR spectrum comprises: a) a PC1 score that is below about −0.20 and a PC2 score that is below about 0.00; b) a PC1 score above about 0.25 and a PC2 score that is below about 0.00; c) a PC1 score from about −0.47 to about −0.20 and a PC2 score that is from about −0.27 to about 0.00; and/or d) a PC1 score from about 0.65 to about 0.9 and a PC2 score that is from about −0.47 to about 0.00; d) a PC1 score that is outside of the range of −0.32 to 0.30 when the PC2 score is above 0.00 and below 0.20; and/or f) a PC2 score is outside the range of 0.00 to 0.20 when the PC1 is above −0.32 and below 0.30.

In some embodiments, the spectroscopic profile indicative of pharmacological and/or clinical efficacy is at least one of a ³¹P-NMR spectrum, a ¹H-NMR spectrum, a ¹³C-NMR spectrum, and a VTCD spectrum. In some embodiments, the ³¹P-NMR spectrum has one or more resonances between 54.8 to 55.8 ppm. In some embodiments, the ³¹P-NMR spectrum has one or more resonances between 54.8 to 55.5 ppm. In some embodiments, the ³¹P-NMR spectrum has two or more resonances between 54.8 to 55.5 ppm. In some embodiments, the ³¹P-NMR spectrum has two or more resonances between 54.8 to 55.5 ppm with different intensities. In some embodiments, the ³¹P-NMR spectrum has one or more resonances between 55.0 to 55.4 ppm In some embodiments, the ³¹P-NMR spectrum has, independently at 54.8 ppm, 54.9 ppm. 55.0 ppm, 55.1 ppm, 55.2 ppm, 55.3 ppm, 55.4 ppm, and/or 55.5 ppm, one or more resonances. In some embodiments, the ³¹P-NMR spectrum has one or more resonances at about 54.8 ppm. In some embodiments, the ³¹P-NMR spectrum has one or more resonances at about 54.9 ppm. In some embodiments, the ³¹P-NMR spectrum has one or more resonances at about 55.0 ppm. In some embodiments, the ³¹P-NMR spectrum has one or more resonances at about 55.1 ppm. In some embodiments, the ³¹P-NMR spectrum has one or more resonances at about 55.2 ppm. In some embodiments, the ³¹P-NMR spectrum has one or more resonances at about 55.3 ppm. In some embodiments, the ³¹P-NMR spectrum has one or more resonances at about 55.4 ppm. In some embodiments, the ³¹P-NMR spectrum has one or more resonances at about 55.5 ppm. In some embodiments, the ³¹P-NMR spectrum has, independently at 54.8 ppm, 54.9 ppm, 55.0 ppm, 55.1 ppm, 55.2 ppm, 55.3 ppm, 55.4 ppm, and/or 55.5 ppm, two or more resonances. In some embodiments, the ³¹P-NMR spectrum has two or more resonances at about 54.8 ppm. In some embodiments, the ³¹P-NMR spectrum has two or more resonances at about 54.9 ppm. In some embodiments, the ³¹P-NMR spectrum has two or more resonances at about 55.0 ppm. In some embodiments, the ³¹P-NMR spectrum has two or more resonances at about 55.1 ppm. In some embodiments, the ³¹P-NMR spectrum has two or more resonances at about 55.2 ppm. In some embodiments, the ³¹P-NMR spectrum has two or more resonances at about 55.3 ppm. In some embodiments, the ³¹P-NMR spectrum has two or more resonances at about 55.4 ppm. In some embodiments, the ³¹P-NMR spectrum has two or more resonances at about 55.5 ppm. In some embodiments, the ³¹P-NMR spectrum, when subjected to PCA, comprises a PC1 score that is below about −0.20 or above about 0.25 and a PC2 score that is below about 0.00. In some embodiments, the ³¹P-NMR spectrum, when subjected to PCA, comprises a PC1 score is outside of the range of −0.32 to 0.31 and/or a PC2 score that is outside the range of 0.00 to 0.20. In some embodiments, the ³¹P-NMR spectrum, when subjected to PCA, comprises a PC1 score that is below about −0.20 and a PC2 score that is below about 0.00. In some embodiments, the ³¹P-NMR spectrum, when subjected to PCA, comprises a PC1 score that is above about 0.25 and a PC2 score that is below about 0.00. In some embodiments, the ³¹P-NMR spectrum, when subjected to PCA, comprises a PC1 score that is from about −0.47 to about −0.20 and a PC2 score that is from about −0.27 to about 0.00. In some embodiments, the ³¹P-NMR spectrum, when subjected to PCA, comprises a PC1 score that is from about 0.65 to about 0.9 and a PC2 score that is from about −0.47 to about 0.00. In some embodiments, the ³¹P-NMR spectrum, when subjected to PCA, comprises a PC1 score that is outside of the range of −0.32 to 0.30 when the PC2 score is above 0.00 and below 0.20. In some embodiments, the ³¹P-NMR spectrum, when subjected to PCA, comprises a PC2 score is outside the range of 0.00 to 0.20 when the PC1 is above −0.32 and below 0.30. In some embodiments, the PCA may be performed as described in Example 7. In some embodiments, the PCA comprises selecting principle components using a modeling data set. In some embodiments, the modeling data set comprises five, six, seven, eight, nine, ten, or up to twenty-seven ³¹P-NMR spectra, e.g., selected from the ³¹P-NMR spectra shown in FIGS. 5C-5CC. In some embodiments, the modeling data comprises ten ³¹P-NMR spectra. e.g., the ten ³¹P-NMR spectra shown in FIGS. 5C, 5L, 5X, 5H, 5P, 5I, 5M, 5T, 5S, and 5BB. In some embodiments, the principal components are selected to account for more than 65%, more than 70%, more than 75%, more than 80%, more than 85%, more than 90%, more than 95%, or more than 99% variance of the modeling data set. In some embodiments, the principal components are selected to account for more than 90% variance of the modeling data set. In some embodiments, the VTCD spectrum has at least one inflection point between 40° C. to 55° C. In some embodiments, the VTCD spectrum has at least one inflection point between 42° C. to 48° C. In some embodiments, the VTCD spectrum has at least one inflection point between 44° C. to 46° C. In some embodiments, the VTCD spectrum has an inflection point at about 45° C.

### Inflammatory Bowel Disease

Disclosed herein, in certain embodiments, are methods of treating, preventing, and/or ameliorating an inflammatory bowel disease or symptoms thereof in a subject in need thereof, the method comprising administering to the subject a pharmaceutical composition comprising a plurality of diastereomers of SMAD7 antisense oligonucleotides (e.g., an antisense oligonucleotide of SEQ ID NO: 1, 2, 3, 4, 5, or 6, or a pharmaceutically acceptable salt thereof), wherein the plurality of diastereomers has a ³¹P-NMR spectrum comprising: a) one or more resonances between 54.8 to 55.5 ppm; and b) (i) a PC1 score that is below about −0.20 or above about 0.25 and a PC2 score that is below about 0.00; or (ii) a PC1 score is outside of the range of −0.32 to 0.31 and/or a PC2 score that is outside the range of 0.00 to 0.20. In some embodiments, provided herein are methods of treating an inflammatory bowel disease, comprising administering a pharmaceutical composition comprising a plurality of diastereomers of SMAD7 antisense oligonucleotides, wherein the plurality of diastereomers has a ³¹P-NMR spectrum comprising: a) one or more resonances between 54.8 to 55.5 ppm; and b) (i) a PC1 score that is below about −0.20 or above about 0.25 and a PC2 score that is below about 0.00; or (ii) a PC1 score is outside of the range of −0.32 to 0.31 and/or a PC2 score that is outside the range of 0.00 to 0.20.

In some embodiments, an inflammatory bowel disease may be Cohn's disease or ulcerative colitis. In some embodiments, an inflammatory bowel disease is Crohn's disease or ulcerative colitis. In some embodiments, an inflammatory bowel disease is Crohn's disease. In some embodiments, an inflammatory bowel disease is ulcerative colitis. In some embodiments, a provided method reduces or alleviates one or more symptoms associated with an inflammatory bowel disease, including but not limited to: abdominal pain, diarrhea, rectal bleeding, severe internal cramps/muscle spasms in the region of the pelvis, weight loss, anemia. In some embodiments, a provided method reduces a Crohn's disease activity index (CDAI) score in a subject.

### Postoperative Recurrence of Crohn's Disease (CD)

Disclosed herein, in certain embodiments, are methods of treating, preventing, and/or ameliorating postoperative CD or symptoms thereof in a subject in need thereof, the method comprising administering to the subject a pharmaceutical composition comprising a plurality of diastereomers of SMAD7 antisense oligonucleotides (e.g., an antisense oligonucleotide of SEQ ID NO: 1, 2, 3, 4, 5, or 6, or a pharmaceutically acceptable salt thereof), wherein the plurality of diastereomers has a ³¹P-NMR spectrum comprising: a) one or more resonances between 54.8 to 55.5 ppm; and b) (i) a PC1 score that is below about −0.20 or above about 0.25 and a PC2 score that is below about 0.00; or (ii) a PC1 score is outside of the range of −0.32 to 0.31 and/or a PC2 score that is outside the range of 0.00 to 0.20.

SMAD7 expression may be upregulated in postoperative CD patients. In some embodiments, a postoperative CD patient may be at any disease phase, e.g., early phase of postoperative CD or recurrence phase of postoperative CD. In various embodiments, early phase of postoperative CD may be any disease phase of CD before recurrence phase of postoperative CD. In some embodiments, early phase of postoperative CD is characterized by the massive infiltration of immune cells secreting high levels of inflammatory cytokines (e.g., Tₕ1 cytokines). In some embodiments, early phase CD (e.g., early phase of postoperative CD) is a stage before the appearance of endoscopic recurrence. Accordingly, in certain embodiments, early phase CD patients may be postoperative CD patients without endoscopic recurrence (e.g., a Rutgeerts score of i0 or i1). In some embodiments, the number of SMAD7-expressing cells in the whole intestinal mucosa, mucosal epithelium and/or lamina propria is greater than the number of cells in later stages of the disease (e.g., established phase CD). As described herein, patients with established phase CD (e.g., recurrence phase of postoperative CD) are patients having chronically active disease. In some embodiments, patients (e.g., postoperative CD patients) with established phase CD may be poorly responsive to medical treatment. In some embodiments, patients (e.g., postoperative CD patients) with established phase CD may benefit from surgical intervention. In certain embodiments, established phase CD can be determined using the Crohn's Disease Activity Index (CDAI) (e.g., CDAI≥150, CDAI≥200, or CDAI≥250) or the Lewis score (LS) (e.g., LS≥700, LS≥750, or LS≥790).

Upregulated SMAD7 expression in a postoperative CD patient may be indicative of postoperative recurrence of CD and/or of the patient being at risk for the recurrence of CD. In some embodiments, the recurrence of CD may be endoscopic recurrence, histological recurrence, radiographic recurrence, clinical recurrence, or a combination thereof. In some embodiments, the recurrence of CD may be endoscopic recurrence. In some embodiments, the endoscopic recurrence may be characterized by one or more signs selected from a group consisting of: lesions (e.g., mucosal lesions), aphthae, ulcers, narrowing of the lumen in the neoterminal ileum, abscesses, fistulas, joint pain, diarrhea, stomach pain or cramping, fever, bloody stool, anemia, and combinations thereof.

The expression of SMAD7 may be determined by methods known in the art for measuring the expression level of SMAD7 mRNA or SMAD7 protein. For example, SMAD7 protein expression level may be quantified by, e.g., immunohistochemistry (IHC), enzyme-linked immunosorbent assay (ELISA), protein immunoprecipitation, immunoelectrophoresis, western blot, protein immunostaining, and spectrometry methods (e.g., chromatography, mass spectrometry). SMAD7 gene expression level may be quantified by, e.g., real-time PCR (quantitative PCR, qPCR), microarray, next-generation sequencing (RNA-Seq), northern blotting, and serial analysis of gene expression (SAGE).

To determine whether SMAD7 expression is upregulated, the expression level of SMAD7 may be compared to a control value. In some embodiments, the control value may be determined by measuring the expression level of SMAD7 in a CD patient prior to a surgical treatment or during a surgical treatment. In some embodiments, the control value may be determined by measuring the expression level of SMAD7 in a subject without CD. In some embodiments, the SMAD7 expression level is at least about 10%, at least about 20%, at least about 30%, at least about 40%, at least about 60%, or at least about 80% elevated as compared to the known control level.

In some embodiments, the upregulation of SMAD7 expression is statistically significant. In some embodiments, the elevation of the SMAD7 expression level in mucosa (e.g., ileal mucosa) of a CD patient as compared to that of a subject without CD is statistically significant. The CD patient may be a postoperative CD patient. In some embodiments, the elevation of the SMAD7 expression level in mucosa (e.g., ileal mucosa) of a postoperative CD patient as compared to that of an established CD patient is statistically significant. Statistical significance can be determined using a standard statistical test, for example, Student's T Test or Mann-Whitney U test.

In some embodiments, the present disclosure provides a method of preventing or treating postoperative recurrence of CD. In some embodiments, the present disclosure provides a method for preventing or treating postoperative recurrence of CD by inhibiting SMAD7. In some embodiments, inhibiting SMAD7 may comprise administering to the subject a pharmaceutical composition comprising a SMAD7 antisense oligonucleotide. In some embodiments, the SMAD7 ASO comprises a nucleobase sequence selected from any one of SEQ ID NOs: 1-6 or a pharmaceutically acceptable salt thereof. In some embodiments, the pharmaceutical composition comprising a plurality of diastereomers of SMAD7 antisense oligonucleotides (e.g., an antisense oligonucleotide of SEQ ID NO: 1, 2, 3, 4, 5, or 6, or a pharmaceutically acceptable salt thereof).

In some embodiments, the pharmaceutical composition may be administered orally. In some embodiments, the pharmaceutical composition may be administered immediately after, about 1 hour after, about 3 hours after, about 6 hours after, about 12 hours after, about 1 day after, about 3 days after, about 1 week after, about 2 weeks after, about 1 month after, about 2 months after, about 3 months after, about 4 months after, about 5 months after, about 6 months after, about 7 months after, about 8 months after, about 9 months after, about 10 months after, about 11 months after, or about 12 months after a surgical treatment for CD the subject receive.

In some embodiments, the present disclosure provides methods of predicting postoperative recurrence of CD in a subject having CD, comprising determining the level of SMAD7 in a sample from the subject, wherein an elevated SMAD7 level relative to a known control level is predictive of the recurrence of CD. Also provided herein are methods of identifying a subject at risk of postoperative recurrence of CD, comprising determining the level of SMAD7 in a sample from the subject, wherein an elevated SMAD7 level relative to a known control level identifies the subject as being at risk for the recurrence of CD. In some embodiments, the known control level of a provided method is the level of SMAD7 in a sample collected from the subject prior to the surgical treatment or during the surgical treatment. In some other embodiments, the known control level is the level of SMAD7 in a healthy subject without CD. In some embodiments, a provided method comprises, if the SMAD7 level is elevated relative to the known control level, administering to the subject a pharmaceutical composition comprising a SMAD7 ASO. In some embodiments, a provided method comprises, if the SMAD7 level is not elevated relative to the known control level, then determining the level of SMAD7 in a second sample from the subject. In some embodiments, a provided method comprises monitoring SMAD7 level in the subject for a predetermined period of time (e.g., about 1 hour, about 3 hours, about 6 hours, about 12 hours, about 1 day, about 3 days, about 1 week, about 2 weeks, about 1 month, about 2 months, about 3 months, about 4 months, about 5 months, about 6 months, about 7 months, about 8 months, about 9 months, about 10 months, about 11 months, or about 12 months after a surgical treatment for CD).

In some embodiments, the subject of a provided method may have received at least one surgical treatment for CD. In some embodiments, the surgical treatment is selected from a group consisting of: bowel resection, ileocolonic resection, colectomy, proctocolectomy, strictureplasty, ileostomy, anal fistulotomy, and combinations thereof. In some embodiments, the surgical treatment is ileocolonic resection.

In some embodiments, the subject of a provided method may exhibit no sign of postoperative recurrence. In some embodiments, the subject exhibits at least one sign of postoperative recurrence. In some embodiments, the postoperative recurrence is selected from the group consisting of: endoscopic recurrence, histological recurrence, radiographic recurrence, clinical recurrence, and combinations thereof. In some embodiments, the subject exhibits at least one sign of endoscopic recurrence. In some embodiments, the sign of postoperative recurrence may be selected from a group consisting of: lesions (e.g., mucosal lesions), aphthae, ulcers, narrowing of the lumen in the neoterminal ileum, abscesses, fistulas, joint pain, diarrhea, stomach pain or cramping, fever, bloody stool, anemia, and combinations thereof. In some embodiments, the sign of postoperative recurrence may be lesions, such as mucosal lesions.

In some embodiments, at least one sample from the subject of a provided method may have an elevated SMAD7 level relative to a known control level. In some embodiments, the known control level of a provided method is the level of SMAD7 in a sample collected from the subject prior to the surgical treatment or during the surgical treatment. In some other embodiments, the known control level is the level of SMAD7 in a healthy subject without CD. In some embodiments, the SMAD7 level may be a SMAD7 mRNA level or SMAD7 protein level. In some embodiments, the SMAD7 level is at least about 10%, at least about 20%, at least about 30%, at least about 40%, at least about 60%, or at least about 80% elevated as compared to the known control level. In some embodiments, a sample from the subject of a provided method may be a tissue sample, such as a skin or mucosal tissue sample. In some embodiments, the sample may be a mucosal tissue sample. In some embodiments, a tissue sample from the subject (e.g., a tissue sample from the small intestine, large intestine, and/or neoterminal ileum of the subject) may be used as a source of cells, a source of RNA, a source of protein, or a source of thin sections for measuring the amount of SMAD7-positive cells in the sample, e.g., using immunohistochemistry (IHC) or flow cytometry. The tissue sample may be obtained using conventional biopsy instruments and procedures, such as endoscopic biopsy, excisional biopsy, or incisional biopsy. The tissue sample may be in any form sufficient for cell sorting, RNA extraction, protein extraction, or preparation of thin sections. Accordingly, the tissue sample may be fresh, preserved through suitable cryogenic techniques, or preserved through non-cryogenic techniques. An exemplary standard process for handling clinical biopsy specimens is to fix the tissue sample in formalin and then embed it in paraffin. Samples in this form are commonly known as formalin-fixed, paraffin-embedded (FFPE) tissue. Suitable techniques of tissue preparation for subsequent analysis are well-known to those of skill in the art. In some embodiments, a sample from the subject may be collected immediately after, about 1 hour after, about 3 hours after, about 6 hours after, about 12 hours after, about 1 day after, about 3 days after, about 1 week after, about 2 weeks after, about 1 month after, about 2 months after, about 3 months after, about 4 months after, about 5 months after, about 6 months after, about 7 months after, about 8 months after, about 9 months after, about 10 months after, about 11 months after, or about 12 months after a surgical treatment for CD.

### Spectroscopic Techniques of the Disclosure

In some embodiments, the present disclosure relates to assessing the spectroscopic profile of oligonucleotides. The spectroscopic profile may be obtained via one or more various spectroscopic methods, e.g., circular dichroism (e.g., variable temperature circular dichroism), fluorescence spectroscopy, mass spectroscopy, nuclear magnetic resonance (NMR) spectroscopy (e.g., Phosphorus-31 NMR, ¹H-NMR, ¹³C-NMR), Raman spectroscopy, ultraviolet-visible spectroscopy, and X-ray photoelectron spectroscopy. In some embodiments, the present disclosure provides methods of evaluating diastereomeric profile an oligonucleotide by assessing its spectroscopic profile. In some embodiments, a spectroscopic profile of an ASO is obtained, wherein “obtaining” refers to performing a spectroscopic analysis and acquiring data of at least one of ³¹P-NMR, ¹H-NMR, ¹³C-NMR, and VTCD, and/or acquiring spectroscopic data of at least one of a ³¹P-NMR analysis, a ¹H-NMR analysis, a ¹³C-NMR analysis, and a VTCD analysis that has already been performed. The present disclosure further provides a method of predicting pharmacological efficacy of an oligonucleotide by assessing its spectroscopic profile. In some embodiments, provided herein is a method of selecting a pharmacologically and/or clinically efficacious oligonucleotide by assessing the spectroscopic profile of the oligonucleotide.

### Phosphorus-31 Nuclear Magnetic Resonance (³¹P-NMR) Spectroscopy

³¹P-NMR spectroscopy is a common analytical chemistry technique that uses nuclear magnetic resonance to study phosphorus-containing molecules, such as probing the structure and dynamics of DNA and DNA fragments. ³¹P-NMR can be very sensitive, as it has a large resonance range, providing specific information of chemical environments of different phosphorus atoms present in a molecule. In addition to the primary information (resonance of the individual ³¹P atom, related to the chemical environment), the fine structure of each peak can provide information on the local conformation of the molecule (via measuring one-bond and three-bond coupling constants).

Phosphorothioate (PS) oligonucleotides have one or more chiral PS centers at the phosphor atom(s) of the phosphorothioate linkage(s). Such a PS oligonucleotide is a mixture of diastereomers. For example, a PS antisense oligonucleotide (ASO) that is a 21-mer manifolds 2²⁰ possible diastereomers at the phosphorus atoms.

When subjecting a PS ASO to ³¹P-NMR spectroscopy (e.g., solution ³¹P-NMR spectroscopy), each diastereomer, bearing 20 phosphorus atoms, is expected to display up to 20 more or less resolved ³¹P NMR lines. Each ³¹P atom is to be found in a similar chemical environment, and the resonance dispersion is modest. However, secondary effects resulting from the different stereochemistry of diastereomers may be probed by examining the ³¹P resonance in high resolution spectra.

In some embodiments, the present disclosure relates to assessing a solution ³¹P-NMR of an oligonucleotide. In some embodiments, the present disclosure provides a method for assessing the diastereomeric profile of an oligonucleotide using solution ³¹P-NMR spectroscopy.

In some embodiments. “resonance.” “signal,” “chemical shift.” “peak,” and “component” are used interchangeably in relation to ³¹P-NMR spectra.

### Proton Nuclear Magnetic Resonance (¹H-NMR) Spectroscopy

¹H-NMR is the application of nuclear magnetic resonance in NMR spectroscopy with respect to hydrogen-1 nuclei within the molecules of a substance, in order to determine the structure of its molecules.

In some embodiments. PS linkages of an oligonucleotide impact the overall stereochemistry and dynamics of an oligonucleotide, and thereby the ¹H-NMR spectrum of the oligonucleotide. In some embodiments, the present disclosure provides a method for assessing the diastereomeric profile of an oligonucleotide using ¹H-NMR spectroscopy.

### Carbon-13 Nuclear Magnetic Resonance (¹³C-NMR) Spectroscopy

¹³C-NMR is the application of nuclear magnetic resonance spectroscopy to carbon. ¹³C-NMR detects only the ¹³C isotope of carbon, whose natural abundance is only 1.1% with respect to hydrogen-1 nuclei within the molecules of a substance. ¹³C resonances follow the same principles as those of ¹H, while the typical range of resonances is much larger than for ¹H. ¹³C-NMR allows the identification of carbon atoms in an organic molecule and is an important tool in chemical structure elucidation, e.g., stereochemical assignment.

In some embodiments, the present disclosure provides a method for assessing the diastereomeric profile of an oligonucleotide using ¹³C-NMR spectroscopy.

### Variable Temperature Circular Dichroism

Circular dichroism (CD), such as variable temperature CD (VTCD), is a useful technique for examining the overall structure of an oligonucleotide. Owing to the different overall stereochemistry, various diastereomeric mixtures of an oligonucleotide may manifest different circular dichroism spectra, as a function of temperature.

In some embodiments, provided herein is a method for assessing the diastereomeric profile of an oligonucleotide using variable temperature circular dichroism (VTCD).

### Chemometric Methods of the Disclosure

Chemometrics is the science of extracting information from chemical systems using mathematical and statistical methods or other data driven means. Chemometrics uses methods frequently employed in core data-analytic disciplines such as multivariate statistics, applied mathematics, and computer science, in order to address problems in. e.g., chemistry, biochemistry, medicine, and biology.

Spectroscopic techniques produce profiles containing a high amount of information, which can profitably be exploited through the use of chemometric methods. For example, the application of chemometric methods to NMR spectra has been reported in relation to the classification, diagnosis, and prognosis of osteoarthritis (WO 2002/085195).

In some embodiments, the present disclosure relates to subjecting the spectroscopic profile of oligonucleotides to chemometric analysis. In some embodiments, the present disclosure provides chemometric methods for evaluating the diastereomeric profile of an oligonucleotide, predicting pharmacological efficacy of an oligonucleotide, and/or selecting a pharmacologically and/or clinically efficacious oligonucleotide by assessing the spectroscopic profile of the oligonucleotide.

Multivariate analysis is a critical facet in chemometrics. Data from spectroscopic experiments are highly multivariate. The structure of these data was found to be conducive to using techniques such as principal components analysis (PCA) and partial least-squares (PLS). PCA is performed by either of the following 2 steps: 1) calculating the data covariance (or correlation) matrix of the original data, or 2) performing eigenvalue decomposition on the covariance matrix, or by singular value decomposition (SVD) of a design matrix.

In some embodiments, the present disclosure relates to subjecting the spectroscopic profile (e.g., the ³¹P-NMR spectra) to PCA, as described in Example 7.

### Multivariate Calibration

Many spectroscopic problems and applications of chemometrics involve calibration. The objective is to develop models (e.g., multivariate regression models) which can be used to predict properties of interest based on measured properties of the spectra. Examples include the development of multivariate models relating spectral responses of a diastereomeric profile to pharmacological and/or clinical efficacy. The process requires a calibration or training data set (reference spectroscopic profiles), which includes reference values for the spectroscopic characteristics of interest for prediction, and the measured attributes that correspond to these properties. For example, one can assemble data from a number of samples that are different mixtures of diastereoisomers of an oligonucleotide, including ³¹P-NMR spectra for each sample and corresponding pharmacological and/or clinical efficacy of that sample. Multivariate calibration techniques, for example, but not limited to, partial-least squares regression, and principal component regression, are then used to construct a mathematical model that relates the spectrum to the pharmacological efficacy, and such a model can be used to efficiently predict the pharmacological and/or clinical efficacy of new samples.

### Classification, Pattern Recognition, Clustering

Supervised multivariate classification techniques are closely related to multivariate calibration techniques in that a calibration or training set is used to develop a mathematical model capable of classifying future samples. The techniques employed in chemometrics are similar to those used in other fields including, but not limited to, multivariate discriminant analysis, logistic regression, neural networks, and regression/classification trees. The use of rank reduction techniques in conjunction with these conventional classification methods is routine in chemometrics, for example discriminant analysis on principal components or partial least squares scores.

In some embodiments, the present disclosure relates to applying pattern recognition to NMR spectroscopy. One of the advantages relates to the intrinsic accuracy in a very complex matrix of potential interferences. In some embodiments, the present disclosure relates to employing multivariate regression modeling (“MRM”) as a means of con-elating spectral data with known compositional changes.

### Pharmaceutical Compositions and Routes of Administration

Pharmaceutical compositions containing an oligonucleotide, such as a SMAD7 ASO described herein, can be presented in a dosage unit form and can be prepared by any suitable method. A pharmaceutical composition should be formulated to be compatible with its intended route of administration. Useful formulations can be prepared by methods well known in the pharmaceutical art. For example, see Remington's Pharmaceutical Sciences, 18th ed. (Mack Publishing Company, 1990).

Pharmaceutical formulations preferably are sterile. Sterilization can be accomplished, for example, by filtration through sterile filtration membranes. Where the composition is lyophilized, filter sterilization can be conducted prior to or following lyophilization and reconstitution.

In some embodiments contemplated herein are compositions suitable for oral delivery (e.g., capsules, tablets, caplets, pills, troches, lozenges, powders, and granules) of an oligonucleotide. Contemplated oligonucleotides, such as SMAD7 antisense nucleotides of SEQ ID NO: 1, 2, 3, 4, 5, or 6, or pharmaceutically acceptable salts thereof may be administered orally.

The formulation of an oligonucleotide (e.g., an oligonucleotide of SEQ ID NO: 1, 2, 3, 4, 5, or 6, or a pharmaceutically acceptable salt thereof) may be an oral pharmaceutical composition. In some embodiments, the oral pharmaceutical composition includes an oligonucleotide (e.g., SEQ ID NO: 1 or a pharmaceutically acceptable salt thereof). In some embodiments, the oral pharmaceutical composition includes a SMAD7 antisense oligonucleotide (e.g., an oligonucleotide of SEQ ID NO: 1, 2, 3, 4, 5, or 6, or a pharmaceutically acceptable salt thereof).

The pharmaceutical formulation of an oligonucleotide (e.g., an oligonucleotide of SEQ ID NO: 1, 2, 3, 4, 5, or 6, or a pharmaceutically acceptable salt thereof) may be a tablet or capsule.

The tablet of an oligonucleotide (e.g., an oligonucleotide of SEQ ID NO: 1, 2, 3, 4, 5, or 6, or a pharmaceutically acceptable salt thereof) may be formulated as a minitablet or a microtablet. The tablet of an oligonucleotide (e.g., an oligonucleotide of SEQ ID NO: 1, 2, 3, 4, 5, or 6, or a pharmaceutically acceptable salt thereof) may include minitablets, microtablets, or granulates. The capsule of an oligonucleotide (e.g., an oligonucleotide of SEQ ID NO: 1, 2, 3, 4, 5, or 6, or a pharmaceutically acceptable salt thereof) may include minitablets, microtablets, or granulates. The oral pharmaceutical composition may be enteric-coated. In certain embodiments, the oral pharmaceutical composition (tablet or capsule) of an oligonucleotide (e.g., an oligonucleotide of SEQ ID NO: 1 or a pharmaceutically acceptable salt thereof) is enteric-coated or includes enteric-coated micropellets, microtablets, minitablets, or granulates. In certain embodiments, the oral pharmaceutical composition (tablet or capsule) of an oligonucleotide (e.g., an oligonucleotide of SEQ ID NO: 1, 2, 3, 4, 5, or 6, or a pharmaceutically acceptable salt thereof) is enteric-coated or includes enteric-coated micropellets, microtablets, minitablets, or granulates.

In certain embodiments, the oral pharmaceutical composition of an oligonucleotide (e.g., an oligonucleotide of SEQ ID NO: 1, 2, 3, 4, 5, or 6, or a pharmaceutically acceptable salt thereof) is not enteric-coated. In certain embodiments, the oral pharmaceutical composition of an oligonucleotide (e.g., an oligonucleotide of SEQ ID NO: 1 or a pharmaceutically acceptable salt thereof) is not enteric-coated. In certain embodiments, the oral pharmaceutical composition of an oligonucleotide (e.g., an oligonucleotide of SEQ ID NO: 1, 2, 3, 4, 5, or 6, or a pharmaceutically acceptable salt thereof) is not enteric-coated.

In some embodiments, the compositions of an oligonucleotide (i.e., a therapy including an oligonucleotide (e.g., an oligonucleotide of SEQ ID NO: 1, 2, 3, 4, 5, or 6, or a pharmaceutically acceptable salt thereof)) may be suitable for oral delivery of an oligonucleotide, e.g., tablets, that include an enteric coating, e.g., a gastro-resistant coating, such that the compositions may deliver the compound to, e.g., the gastrointestinal tract of a patient. Such administration may result in a topical effect, for example, by substantially topically applying the antisense compound directly to an affected portion of the gastrointestinal tract of a subject. Such administration, may, in some embodiments, substantially avoid unwanted systemic absorption of the antisense compound.

For example, an oral dosage form (e.g., tablet) for oral administration may include granules (e.g., an oral dosage form at least partially formed from granules) that include a disclosed antisense compound (e.g., an oligonucleotide of SEQ ID NO: 1, 2, 3, 4, 5, or 6, or a pharmaceutically acceptable salt thereof) and pharmaceutically acceptable excipients. Such an oral dosage form, e.g., a tablet, may be coated with an enteric coating. Contemplated oral dosage forms, e.g., tablets, may include pharmaceutically acceptable excipients such as fillers, binders, disintegrants, and/or lubricants, as well as coloring agents, release agents, coating agents, sweetening, flavoring such as wintergreen, orange, xylitol, sorbitol, fructose, and maltodextrin, and perfuming agents, preservatives and/or antioxidants.

In some embodiments, contemplated oral dosage forms of the pharmaceutical formulations include an intra-granular phase that includes a contemplated antisense oligonucleotide (e.g., an antisense oligonucleotide of SEQ ID NO: 1, 2, 3, 4, 5, or 6, or a pharmaceutically acceptable salt thereof) and a pharmaceutically acceptable filler. For example, an oligonucleotide (e.g., an oligonucleotide of SEQ ID NO: 1, 2, 3, 4, 5, or 6, or a pharmaceutically acceptable salt thereof) and a filler may be blended together, with optionally other excipients, and formed into granules. In some embodiments, the intragranular phase may be formed using wet granulation, e.g., a liquid (e.g., water) is added to the blended antisense compound and filler, and then the combination is dried, milled and/or sieved to produce granules. Other processes in the art may be used to achieve an intragranular phase.

In some embodiments, contemplated oral dosage forms of the formulations include an extra-granular phase, which may include one or more pharmaceutically acceptable excipients, and which may be blended with the intragranular phase to form a disclosed formulation.

An oligonucleotide (e.g., an oligonucleotide of SEQ ID NO: 1, 2, 3, 4, 5, or 6, or a pharmaceutically acceptable salt thereof) formulation (e.g., oral dosage form) may include an intragranular phase that includes a filler. Exemplary fillers include, but are not limited to, cellulose, gelatin, calcium phosphate, lactose, sucrose, glucose, mannitol, sorbitol, microcrystalline cellulose, pectin, polyacrylates, dextrose, cellulose acetate, hydroxypropylmethyl cellulose, partially pregelatinized starch, calcium carbonate, and others including combinations thereof.

In some embodiments, an oligonucleotide (e.g., an oligonucleotide of SEQ ID NO: 1, 2, 3, 4, 5, or 6, or a pharmaceutically acceptable salt thereof) formulation (e.g., oral dosage form) may include an intragranular phase and/or an extra-granular phase that includes a binder, which may generally function to hold the ingredients of the pharmaceutical formulation together. Exemplary binders include, but are not limited to, the following: starches, sugars, cellulose or modified cellulose such as hydroxypropyl cellulose, lactose, pregelatinized maize starch, polyvinyl pyrrolidone, hydroxypropyl cellulose, hydroxypropylmethyl cellulose, low substituted hydroxypropyl cellulose, sodium carboxymethyl cellulose, methyl cellulose, ethyl cellulose, sugar alcohols and others including combinations thereof.

Contemplated oligonucleotide (e.g., an oligonucleotide of SEQ ID NO: 1, 2, 3, 4, 5, or 6, or a pharmaceutically acceptable salt thereof) formulations (e.g., oral dosage form), e.g., that include an intragranular phase and/or an extra-granular phase, may include a disintegrant such as but not limited to, starch, cellulose, crosslinked polyvinyl pyrrolidone, sodium starch glycolate, sodium carboxymethyl cellulose, alginates, corn starch, crosmellose sodium, crosslinked carboxymethyl cellulose, low substituted hydroxypropyl cellulose, acacia, and others including combinations thereof. For example, an intragranular phase and/or an extra-granular phase may include a disintegrant.

In certain embodiments, a contemplated oligonucleotide (e.g., an oligonucleotide of SEQ ID NO: 1, 2, 3, 4, 5, or 6, or a pharmaceutically acceptable salt thereof) formulation includes an intra-granular phase including a disclosed antisense oligonucleotide and excipients chosen from: mannitol, microcrystalline cellulose, hydroxypropylmethyl cellulose, and sodium starch glycolate or combinations thereof, and an extra-granular phase including one or more of: microcrystalline cellulose, sodium starch glycolate, and magnesium stearate or mixtures thereof.

In certain embodiments, a contemplated oligonucleotide (e.g., an oligonucleotide of SEQ ID NO: 1, 2, 3, 4, 5, or 6, or a pharmaceutically acceptable salt thereof) formulation may include a lubricant, e.g., an extra-granular phase may contain a lubricant. Lubricants include but are not limited to talc, silica, fats, stearin, magnesium stearate, calcium phosphate, silicone dioxide, calcium silicate, calcium phosphate, colloidal silicon dioxide, metallic stearates, hydrogenated vegetable oil, corn starch, sodium benzoate, polyethylene glycols, sodium acetate, calcium stearate, sodium lauryl sulfate, sodium chloride, magnesium lauryl sulfate, talc, and stearic acid.

In certain embodiments, pharmaceutical formulations of the present disclosure include an enteric coating. Generally, enteric coatings create a barrier for the oral medication that controls the location at which the drug is absorbed along the digestive tract. Enteric coatings may include a polymer that disintegrates at different rates according to pH. Enteric coatings may include, for example, cellulose acetate phthalate, methyl acrylate-methacrylic acid copolymers, cellulose acetate succinate, hydroxylpropylmethyl cellulose phthalate, methyl methacrylate-methacrylic acid copolymers, ethylacrylate-methacrylic acid copolymers, methacrylic acid copolymer type C, polyvinyl acetate-phthalate, and cellulose acetate phthalate.

In certain embodiments, the enteric coating includes an anionic, cationic, or neutral copolymer based on methacrylic acid, methacrylic/acrylic esters or their derivatives. In certain embodiments, the enteric coating includes an ethylacrylate-methacrylic acid copolymer. Commercially available enteric coatings include Opadry® AMB, ethylacrylate-methacrylic acid copolymers (e.g., Acryl-EZE®), dimethylaminoethyl methacrylate-butyl methacrylate-methyl methacrylate copolymer (2:1:1), or poly(methacrylic acid-co-methyl-methacrylate) 1:1 and poly(methacrylic acid-co-methyl-methacrylate) 1:2 copolymers (e.g., Eudragit®) grades. In some embodiments, the enteric coating makes up about 5% to about 10%, about 5% to about 20%, about 8 to about 15%, about 8% to about 18%, about 10% to about 12%, or about 12% to about 16%, of a contemplated pharmaceutical composition (e.g., a tablet) by weight. In some embodiments, the enteric coating comprises an ethylacrylate-methacrylic acid copolymer that makes up about 8 to about 15% of the pharmaceutical composition (e.g., tablet) by weight. In some embodiments, the enteric coating comprises an ethylacrylate-methacrylic acid copolymer that makes up about 12% of the pharmaceutical composition (e.g., tablet) by weight. In some embodiments, the enteric coating comprises an ethylacrylate-methacrylic acid copolymer that makes up about 10% of the pharmaceutical composition (e.g., tablet) by weight.

In some embodiments, an oligonucleotide (e.g., an antisense oligonucleotide of SEQ ID NO: 1, 2, 3, 4, 5, or 6, or a pharmaceutically acceptable salt thereof) in the form of a tablet is provided that includes about 0.5% to about 70%, e.g., about 0.5% to about 30%, about 1% to about 20%, or about 5% to about 30% by weight of an antisense oligonucleotide or a pharmaceutically acceptable salt thereof. Such a tablet may include, in some embodiments, about 0.5% to about 60% by weight of mannitol, e.g., about 20% to about 50% by weight mannitol. e.g., about 40% or about 28% by weight mannitol; and/or about 20% to about 40% by weight of microcrystalline cellulose, or about 10% to about 30% by weight of microcrystalline cellulose. In some embodiments, a contemplated tablet may include an intragranular phase that includes about 30% to about 60%, about 45% to about 65% by weight, or alternatively, about 5% to about 10% by weight of an oligonucleotide of SEQ ID NO: 1, 2, 3, 4, 5, or 6, or a pharmaceutically acceptable salt thereof, about 30% to about 50%, or alternatively, about 5% to about 15% by weight mannitol, about 5% to about 15% microcrystalline cellulose, about 0% to about 4%, or about 1% to about 7% hydroxypropyl methylcellulose, and about 0% to about 4%, e.g., about 2% to about 4% sodium starch glycolate by weight. In some embodiments, a contemplated tablet may include about 5% to about 10% or about 10% to about 30% by weight of an oligonucleotide of SEQ ID NO: 1, about 20% to about 50% by weight mannitol, about 10% to about 30% by weight microcrystalline cellulose, about 0.5% to about 10% by weight hydroxypropyl methylcellulose, and about 0.5% to about 10% by weight sodium starch glycolate by weight.

Exemplary oligonucleotide formulations include dosage forms that include or consist essentially of about 10 mg to about 500 mg of an oligonucleotide of SEQ ID NO: 1, 2, 3, 4, 5, or 6, or a pharmaceutically acceptable salt thereof, for example, tablets that include about 10 mg, about 15 mg, about 20 mg, about 25 mg, about 30 mg, about 35 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, or about 250 mg of an oligonucleotide of SEQ ID NO: 1, 2, 3, 4, 5, or 6, or a pharmaceutically acceptable salt thereof, are contemplated herein. In some embodiments, pharmaceutical compositions described herein are formulated as tablets comprising about 10 mg, about 15 mg, about 20 mg, about 25 mg, about 30 mg, about 35 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, or about 250 mg of an oligonucleotide of SEQ ID NO: 1. In some embodiments, pharmaceutical compositions described herein are formulated as tablets comprising about 40 mg of an oligonucleotide of SEQ ID NO: 1. In some embodiments, pharmaceutical compositions described herein are formulated as tablets comprising about 160 mg of an oligonucleotide of SEQ ID NO: 1. In certain embodiments, pharmaceutical compositions described herein are formulated as tablets for oral use, including: about 0.5% to about 30% by weight of the oligonucleotide; about 20% to about 50% by weight mannitol; and about 10% to about 30% by weight microcrystalline cellulose.

In some embodiments, a pharmaceutically acceptable tablet for oral administration is provided that includes about 5% to about 30% by weight of an oligonucleotide of SEQ ID NO: 1, 20% to about 50% by weight of mannitol, about 10% to about 30% by weight of microcrystalline cellulose, about 0.5% to about 10% by weight of hydroxypropyl methylcellulose, and about 0.5% to about 10% by weight of sodium starch glycolate, about 0.5% to about 10% by weight of magnesium stearate, about 0.5% to about 10% by weight Opadry® AMB, and about 5% to about 20% by weight Acryl-EZE®. In some embodiments, a pharmaceutically acceptable tablet for oral administration is provided that includes about 8.5% by weight of an oligonucleotide of SEQ ID NO: 1, 40% by weight of mannitol, about 25% by weight of microcrystalline cellulose, about 5% by weight of hydroxypropyl methylcellulose, about 4% by weight of sodium starch glycolate, about 0.4% by weight of magnesium stearate, about 4% by weight Opadry® AMB, and about 10% to about 15% by weight Acryl-EZE®. In some embodiments, a pharmaceutically acceptable tablet for oral administration is provided that includes about 23% by weight of an oligonucleotide of SEQ ID NO: 1, 28% by weight of mannitol, about 25% by weight of microcrystalline cellulose, about 5% by weight of hydroxypropyl methylcellulose, about 4% by weight of sodium starch glycolate, about 0.4% by weight of magnesium stearate, about 4% by weight Opadry® AMB, and about 7% to about 12% by weight Acryl-EZE®. In some embodiments, a pharmaceutically acceptable tablet for oral administration may be formulated according to Table 0-1 and Table 0-2.

TABLE 0-1

| Component | Amount (mg) |
|---|---|
| SEQ ID NO: 1 | 40 |
| Mannitol | 195.3 |
| Microcrystalline Cellulose | 118.7 |
| Hydroxypropyl | 24 |
| methylcellulose | |
| Sodium starch glycolate | 20 |
| Magnesium stearate | 2 |
| Opadry AMB White | 20 |
| Acryl−EZE clear | 58.8 |
| Total Tablet Weight | 478.8 |

TABLE 0-2

| Component | Amount (mg) |
|---|---|
| SEQ ID NO: 1 | 160 |
| Mannitol | 193 |
| Microcrystalline Cellulose | 178 |
| Hydroxypropyl | 36 |
| methylcellulose | |
| Sodium starch glycolate | 30 |
| Magnesium stearate | 3 |
| Opadry AMB White | 30 |
| Acryl-EZE clear | 69.3 |
| Total Tablet Weight | 699.3 |

In an exemplary embodiment of the disclosure, a pharmaceutically acceptable tablet for oral administration is provided that includes an intra-granular phase that may include about 50% by weight of an oligonucleotide of SEQ ID NO: 1, 2, 3, 4, 5, or 6, or a pharmaceutically acceptable salt thereof, about 11.5% by weight mannitol, about 10% by weight microcrystalline cellulose, about 3% by weight hydroxypropyl methylcellulose, and about 2.5% by weight sodium starch glycolate; and an extra-granular phase that may include about 20% by weight microcrystalline cellulose, about 2.5% by weight sodium starch glycolate, and about 0.5% by weight magnesium stearate. The tablet may also include an enteric coating.

In another exemplary embodiment, a pharmaceutically acceptable tablet for oral administration is provided that includes or consists essentially of: an intra-granular phase that may include about 5% to about 10%, e.g., about 8% by weight of an oligonucleotide of SEQ ID NO: 1, 2, 3, 4, 5, or 6, or a pharmaceutically acceptable salt thereof (e.g., a sodium salt), about 40% by weight mannitol, about 8% by weight microcrystalline cellulose, about 5% by weight hydroxypropyl methylcellulose, and about 2% by weight sodium starch glycolate; and an extra-granular phase that may include about 17% by weight microcrystalline cellulose, about 2% by weight sodium starch glycolate, and about 0.4% by weight magnesium stearate.

Contemplated tablets may also include an enteric coating. e.g., a disclosed tablet may include about 13%, about 14%, about 15%, about 16%, or about 17% by weight of an enteric coating, e.g., ethylacrylate-methacrylic acid copolymers (e.g., AcrylEZE®).

For example, the oligonucleotide may be in the form of a pharmaceutically acceptable tablet for oral use including an intra-granular phase and extra-granular phase, in which for example, the intra-granular phase includes about 5% to about 10%, by weight (for example about 8% by weight) of an oligonucleotide represented by SEQ ID NO: 1, 2, 3, 4, 5, or 6, or a pharmaceutically acceptable salt thereof, about 40% by weight mannitol, about 8% by weight microcrystalline cellulose, about 5% by weight hydroxypropyl methylcellulose, and about 2% by weight sodium starch glycolate, and for example, the extra-granular phase includes about 17% by weight microcrystalline cellulose, about 2% by weight sodium starch glycolate, and about 0.4% by weight magnesium stearate, where the tablet may further include an enteric coating.

Contemplated formulations, e.g., tablets, in some embodiments, when orally administered to the patient may result in minimal plasma concentration of the antisense oligonucleotide in the patient. In another embodiment, contemplated formulations, when orally administered to a patient, topically deliver to the terminal ileum and/or right colon of a patient, e.g., to an affected or diseased intestinal site of a patient. Formulations suitable for oral administration may be in the form of capsules, cachets, pills, tablets, lozenges (using. e.g., a flavored basis such as sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia), each containing a predetermined amount of a subject composition thereof as an active ingredient. Compositions of the present disclosure may also be administered as a bolus, electuary, or paste.

In certain embodiments, a pharmaceutical oral dosage form (e.g., tablet formulation) of an oligonucleotide may include an intra-granular phase, where the intra-granular phase includes an antisense oligonucleotide such as an oligonucleotide of SEQ ID NO: 1, 2, 3, 4, 5, or 6, or a pharmaceutically acceptable salt thereof (e.g., a sodium salt), and a pharmaceutically acceptable filler, and which may also include an extra-granular phase, that may include a pharmaceutically acceptable excipient such as a disintegrant. The extra-granular phase may include components chosen from microcrystalline cellulose, magnesium stearate, and mixtures thereof. The pharmaceutical composition may also include an enteric coating of about 12% to 16% by weight of the tablet.

In some embodiments, a pharmaceutically acceptable tablet for oral use comprises about 0.5% to about 10% by weight of an antisense oligonucleotide, e.g., an oligonucleotide of SEQ ID NO: 1, 2, 3, 4, 5, or 6, or a pharmaceutically acceptable salt thereof, about 30% to 50% by weight mannitol, about 10% to 30% by weight microcrystalline cellulose, and an enteric coating including an ethylacrylate-methacrylic acid copolymer.

In some embodiments, a pharmaceutically acceptable tablet for oral use comprises an intra-granular phase, including about 5% to about 10% by weight of an antisense oligonucleotide, e.g., an oligonucleotide of SEQ ID NO: 1, 2, 3, 4, 5, or 6, or a pharmaceutically acceptable salt thereof, about 40% by weight mannitol, about 8% by weight microcrystalline cellulose, about 5% by weight hydropropylmethyl cellulose, and about 2% by weight sodium starch glycolate; an extra-granular phase including about 17% by weight microcrystalline cellulose, about 2% by weight sodium starch glycolate, about 0.4% by weight magnesium stearate; and an enteric coating over the tablet including an ethylacrylate-methacrylic acid copolymer.

In some embodiments, the pharmaceutical composition comprises an enteric coating including about 13%, about 15%, about 16%, about 17% or about 18% by weight, e.g., AcyrlEZE® (see, e.g., PCT Publication No. WO2010/054826).

The rate at which point the coating dissolves and the active ingredient is released is its dissolution rate. In an embodiment, a contemplated tablet may have a dissolution profile, e.g., when tested in a USP/EP Type 2 apparatus (paddle) at 100 rpm and 37° C. in a phosphate buffer with a pH of 7.2, of about 50% to about 100% of the oligonucleotide releasing after about 120 minutes to about 240 minutes, for example after 180 minutes. In another embodiment, a contemplated tablet may have a dissolution profile, e.g., when tested in a USP/EP Type 2 apparatus (paddle) at 100 rpm and 37° C. in diluted HCl with a pH of 1.0, where substantially none of the oligonucleotide is released after 120 minutes. A contemplated tablet, in another embodiment, may have a dissolution profile, e.g. when tested in USP/EP Type 2 apparatus (paddle) at 100 rpm and 37° C. in a phosphate buffer with a pH of 6.6, of about 10% to about 30%, or not more than about 50%, of the oligonucleotide releasing after 30 minutes.

In some embodiments, disclosed formulations. e.g. tablets, when orally administered to the patient results in minimal plasma concentration of the oligonucleotide in the patient. In some embodiments, disclosed formulations, when orally administered to a patient, topically deliver to the colon or rectum of a patient. e.g., to an affected or diseased site of a patient.

### Dosage Regimen

The formulation of an oligonucleotide (e.g., an antisense oligonucleotide of SEQ ID NO: 1, 2, 3, 4, 5, or 6) or a pharmaceutically acceptable salt thereof, provided in the present disclosure may be administered or is suitable for administration before and/or after symptoms of an inflammatory bowel disease (e.g., Crohn's disease and ulcerative colitis) is developed.

The formulation of the present disclosure (including e.g., SEQ ID NO: 1, 2, 3, 4, 5, or 6 or a pharmaceutically acceptable salt thereof) may be administered or is suitable for administration about every 6 hours, about every 12 hours, about every 24 hours, about every 48 hours, about every 72 hours, every day, two-times a week, once in 2 weeks, or once a month.

Dosing frequency can vary, depending on factors such as mute of administration, dosage amount and the disease being treated. Exemplary dosing frequencies are once per day, once per week and once every two weeks. In certain embodiments, dosing is once per day for 7 days.

In certain embodiments, formulations include dosage forms that include or consist essentially of about 35 mg to about 500 mg of an oligonucleotide (e.g., an oligonucleotide of SEQ ID NO: 1, 2, 3, 4, 5, or 6 or a pharmaceutically acceptable salt thereof). For example, formulations that include about 35 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, or about 250 mg of an oligonucleotide (e.g., an oligonucleotide of SEQ ID NO: 1, 2, 3, 4, 5, or 6 or a pharmaceutically acceptable salt thereof) are contemplated herein. In certain embodiments, a formulation may include about 40 mg, about 80 mg, or about 160 mg of an oligonucleotide such as an oligonucleotide of SEQ ID NO: 1, 2, 3, 4, 5, or 6. In certain embodiments, a formulation may include at least about 100 μg of an oligonucleotide such as an oligonucleotide of SEQ ID NO: 1, 2, 3, 4, 5, or 6. For example, formulations may include about 0.1 mg, about 0.2 mg, about 0.3 mg, about 0.4 mg, about 0.5 mg, about 1 mg, about 5 mg, about 10 mg, about 15 mg, about 20 mg, or about 25 mg of an oligonucleotide such as an oligonucleotide of SEQ ID NO: 1, 2, 3, 4, 5, or 6.

The amount administered will depend on variables such as the type and extent of disease or indication to be treated, the overall health and size of the patient, the in vivo potency of the oligonucleotide, the pharmaceutical formulation, and the route of administration. The initial dosage can be increased beyond the upper level in order to rapidly achieve the desired blood-level or tissue level. Alternatively, the initial dosage can be smaller than the optimum, and the dosage may be progressively increased during the course of treatment. Human dosage can be optimized, e.g., in a conventional Phase I dose escalation study designed to run from 40 mg to 160 mg.

In some embodiments, a patient having an inflammatory bowel disease. e.g., Crohn's disease or ulcerative colitis, will be administered an initial dose of an SMAD7 antisense oligonucleotide, for instance, a SMAD7 antisense oligonucleotide of SEQ ID NO: 1. As used herein, “initial dose” refers to a dose of an SMAD7 antisense oligonucleotide administered to a patient having an inflammatory bowel disease, e.g., Crohn's disease or ulcerative colitis, in a series of doses. A series of doses may include one or more doses. For instance, a series of doses may include a single dose of an SMAD7 antisense oligonucleotide or more than a single dose of an SMAD7 antisense oligonucleotide. An initial dose may be a dose of an SMAD7 antisense oligonucleotide administered to a patient prior to any later dose administered to the patient. For instance, an initial dose may be, but is not limited to, the first dose of an SMAD7 antisense oligonucleotide administered to a treatment-naïve patient. An initial dose may also be a first dose in any treatment cycle of the SMAD7 antisense oligonucleotide. For example, an initial dose may be the first dose of a first treatment cycle, of a second treatment cycle, or of any subsequent treatment cycles.

In some embodiments, a patient having an inflammatory bowel disease, e.g., Crohn's disease or ulcerative colitis, may be administered a subsequent dose of a SMAD7 antisense oligonucleotide, for instance, a SMAD7 antisense oligonucleotide of SEQ ID NO: 1. As used herein, “subsequent dose” refers to a dose of an SMAD7 antisense oligonucleotide administered to a patient having inflammatory bowel disease, e.g., Crohn's disease or ulcerative colitis, after administration of a prior dose, for example, an initial dose. Thus, a subsequent dose may be administered to a patient having an inflammatory bowel disease, e.g., Crohn's disease or ulcerative colitis, in a series of doses including two or more doses. Furthermore, in some instances, the amount of a subsequent dose may be calibrated with respect to an initial dose or a prior dose, such that a subsequent dose is greater, equal to, or lesser than a prior dose. A subsequent dose may be a dose administered to a patient having an inflammatory bowel disease, e.g., Crohn's disease or ulcerative colitis, after a first dose, for instance, an initial dose, of an SMAD7 antisense oligonucleotide administered to the patient. A subsequent dose may also be a dose administered after a prior dose of an SMAD7 antisense oligonucleotide administered to a patient having an inflammatory bowel disease. e.g., Crohn's disease or ulcerative colitis, for instance, a dose administered after a prior dose in the same round of treatment or a different round of treatment, for instance, a previous round of treatment. A subsequent dose may be a subsequent dose with respect to any prior dose, for instance, a prior dose immediately preceding the subsequent dose or a prior dose followed by one or more doses administered prior to administration of the subsequent dose.

In some embodiments, the patient having an inflammatory bowel disease, e.g., Crohn's disease has at least about a 5%, about a 10%, about a 20%, about a 30%, about a 40% or even about a 50% or more reduction in the Crohn's Disease Activity Index (CDAI) after administering a SMAD7 antisense oligonucleotide, e.g., a SMAD7 antisense oligonucleotide of SEQ ID NO: 1, 2, 3, 4, 5, or 6 or a pharmaceutically acceptable salt thereof, after e.g., 1 day, 2 days, 1 week, 1 month, or 6 months, or more. Administering a SMAD7 antisense oligonucleotide may be on, e.g., at least a daily basis. The delay of clinical manifestation of an inflammatory bowel disease, e.g., Crohn's disease or ulcerative colitis, in a patient as a consequence of administering a SMAD7 antisense oligonucleotide may be at least, e.g., 6 months, 1 year, 18 months or even 2 years or more as compared to a patient who is not administered a SMAD7 antisense oligonucleotide.

The composition or formulation or method the present disclosure provides administration of an oligonucleotide (e.g., an oligonucleotide of SEQ ID NO: 1, 2, 3, 4, 5, or 6) or a pharmaceutically acceptable salt thereof, to a subject who is refractory to a first therapy.

In some embodiments, a subject who is refractory to the first treatment is treated with an oligonucleotide (e.g., an antisense oligonucleotide of SEQ ID NO: 1, 2, 3, 4, 5, or 6) or a pharmaceutically acceptable salt thereof, concurrently or subsequent to the first therapy.

In some embodiments, methods provided herein may further include administering at least one other agent that is directed to treatment of diseases and disorders disclosed herein (e.g., Crohn's disease or ulcerative colitis). In certain embodiments, contemplated other agents may be co-administered (e.g., sequentially or simultaneously).

Agents contemplated include immunosuppressive agents including glucocorticoids, cytostatics, antibodies, agents acting on immunophilins, interferons, opioids, TNF binding proteins, mycophenolate, and small biological agents. For example, contemplated immunosuppressive agents include, but are not limited to: tacrolimus, cyclosporine, pimecrolimus, sirolimus, everolimus, mycophenolic acid, fingolimod, dexamethasone, fludarabine, cyclophosphamide, methotrexate, azathioprine, leflunomide, teriflunomide, anakinra, anti-thymocyte globulin, anti-lymphocyte globulin, muromonab-CD3, afutuzumab, rituximab, teplizumab, efalizumab, daclizumab, basiliximab, adalimumab, infliximab, and etanercept.

In some embodiments, other contemplated agents may be Toll-like receptor (TLR) modulators or TLR pathway modulators. In some embodiments, the TLR modulator may modulate the activity of TLR3, TLR7, TLR8, and/or TLR9. In some embodiments, the TLR pathway modulator may modulate the activity of one or more components of a TLR pathway, including, but not limited to, C—C motif chemokine receptor 7 (CCR7), cluster of differentiation (CD) 80, CD83, CD86, CD69, epithelial growth factor receptor (EGFR), glycoprotein A repetitions predominant (GARP), interleukin (IL)-1-β, IL-2, IL-10Rα, IL-18, IL-23p19, macrophage inflammatory protein-1 alpha (MIP-la), phospho-histone H3, phospho-p38 mitogen-activated protein MAP kinase, phospho-zeta-chain-associated protein kinase 70 (phospho-ZAP70), receptor activator of nuclear factor kappa-B ligand (RANKL), SLAM family member (SLAMF7), tissue plasminogen activator (tPA), and urokinase receptor (uPAR). Exemplary TLR modulators include, but are not limited to, BL-7040 (ODN7040), CYT003, CYT003-QhG10, AZD1419, DIMS0150 (ODN150), E6446. CpG ODN2088. IMO-8400, IMO-3100. CL075, VTX-2337, ODN2006, and naltrexone. In some embodiments, a TLR modulator or a TLR pathway modulator may be an agonist. In some other embodiments, a TLR modulator or a TLR pathway modulator may be an antagonist.

### EXAMPLES

The disclosure is further illustrated by the following examples. The examples are provided for illustrative purposes only, and are not to be construed as limiting the scope or content of the disclosure in any way.

### Example 1: Preparation of SMAD7 Antisense Oligonucleotides

27 batches—Batches A-Z and S20—of a SMAD7 antisense oligonucleotide (ASO) (SEQ ID NO: 1) were manufactured following solid-phase, non-chirality controlled synthetic protocols, at different scales and under different conditions.

Each batch of the SMAD7 ASO resulting from the non-chirality controlled synthesis is a mixture of diastereomers with unknown individual levels of up to 2′° (i.e., 1,048,576) possible different diastereomers.

### Example 2: In Vitro Activity of SMAD7 Antisense Oligonucleotide Diastereomeric Mixtures

To assess in vitro pharmacological activity of SMAD7 ASO with different diastereomeric profiles, the capability of different batches of SMAD7 ASO (Batches A-Q, S-Z, and S20) to down-regulate SMAD7 expression was investigated in the HCT-116 cell line.

SMAD7 ASO batches in powder form were dissolved in phosphate buffered saline (PBS) and concentration determined using reverse phase high-performance liquid chromatography (HPLC).

Human colorectal carcinoma cell line HCT-116 (American Type Culture Collection, ATCC) were maintained in McCoy's 5A (Lonza) supplemented with 10% fetal bovine serum (FBS) (Euroclone) and 1% penicillin/streptomycin (P/S) (Lonza), in a 37° C., 5% CO₂, fully humidified incubator.

Single-cell suspensions were plated at 2×10⁵ cells/mL/well in 6-well culture dishes and allowed to adhere overnight. On the following day, cells were washed twice with PBS (Lonza) and then transfected with the test items—SMAD7 ASO Batches A-Q, S-Z, and S20—at 0.5 or 1 μg/mL in PBS, using Opti-MEM I transfection medium and Lipofectamine 3000 reagent (Life Technologies) according to the manufacturer's instructions. Transfected cells were then washed with PBS and cultured with McCoy's 5A supplemented with 10% FBS and antibiotics. Cells transfected with Batch A were used as reference (positive control). Cells untransfected but treated with transfection medium and Lipofectamine 3000 were used as negative control.

After 24 hours, cells were then washed with PBS and total proteins extracted using the following lysis buffer: 10 mmol/L HEPES, 1 mmol/L EDTA, 60 mmol/L KCl, 0.2% Igepal CA-630, 1 mmol/L sodium fluoride, 10 μg/ml aprotinin, 10 μg/ml leupeptin, 1 mmol/L DTT, and 1 mmol/L PMSF (Sigma-Aldrich). The lysates were separated on a 10% SDS-PAGE gel. Blots were then incubated with a mouse anti-human monoclonal antibody against the target protein at 0.5 μg/mL (R&D systems) followed by a rabbit anti-mouse antibody conjugated to horseradish peroxidase (1:20000 dilution, Dako). After analysis of the target protein SMAD7, each blot was stripped and incubated with a mouse-anti-human monoclonal β-actin antibody (Sigma-Aldrich) to confirm equal loading of the lanes. Computer-assisted scanning densitometry (Image Lab Software. Bio-Rad Laboratories) was used to analyze the intensity of the immunoreactive bands and, thereby, quantify the SMAD7/3-actin protein level ratio.

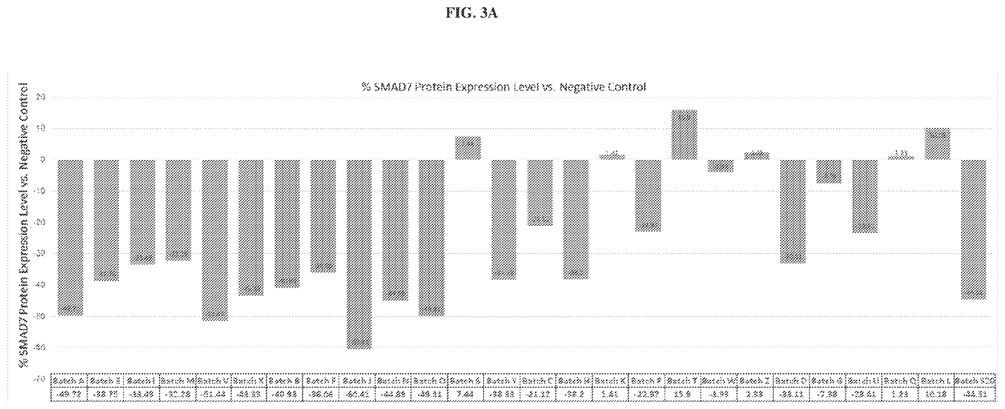

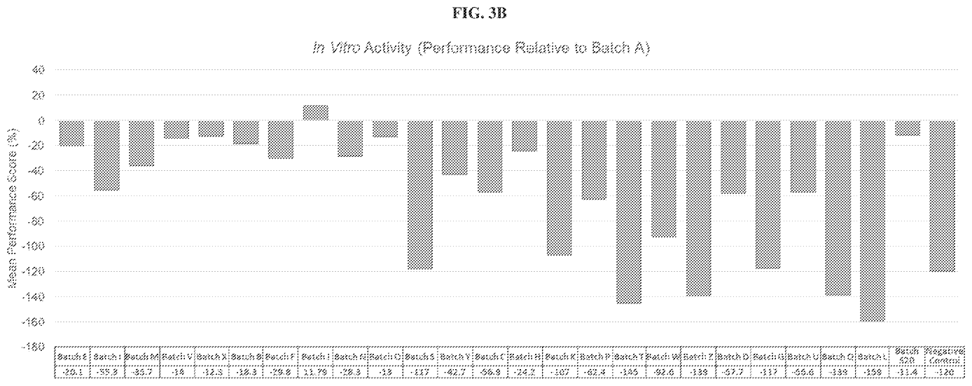

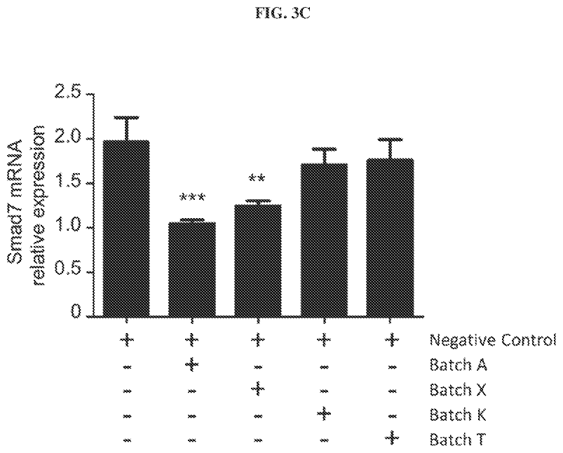

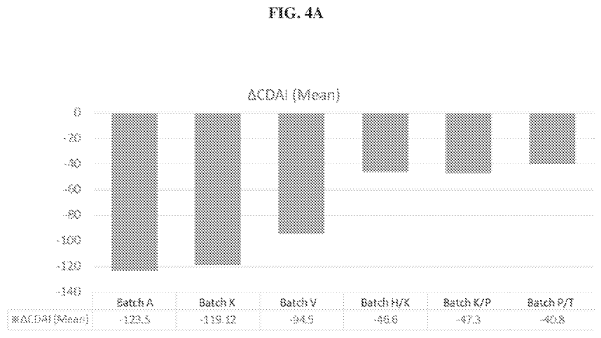

Exemplary results are illustrated in FIG. 3A and Table 1, comparing the change in the SMAD7/β-actin ratio upon transfection of cells with each tested batch to the SMAD7/β-actin ratio of cells treated with Lipofectamine 3000 only (negative control). Values were expressed as mean±S.E.M of at least three independent experiments. Data were analyzed using one-way analysis of variance (ANOVA) followed by Dunnett's Multiple Comparison Test. Significance was defined as P-values (n.s.=not significant; **=p≤0.01; ***=p≤0.001). In certain embodiments, a more than about 40% down-regulation of SMAD7 protein expression compared to cells treated with Lipofectamine 3000 alone (negative control) indicates good in vitro activity of the tested SMAD7 ASO batch.

TABLE 1
Mean percentage difference in SMAD7 protein expression
in HCT-116 cells transfected with SMAD7 ASO batches
as compared to cells treated with Lipofectamine 3000

| | % SMAD7 |
|---|---|
| Batch | Expression |
| Batch A | −49.72 |
| Batch B | −40.93 |
| Batch C | −21.12 |
| Batch D | −33.11 |
| Batch E | −38.75 |
| Batch F | −36.06 |
| Batch G | −7.38 |
| Batch H | −38.2 |
| Batch I | −33.49 |
| Batch J | −60.41 |
| Batch K | 1.41 |
| Batch L | 10.18 |
| Batch M | −32.28 |
| Batch N | −44.89 |
| Batch O | −49.81 |
| Batch P | −22.97 |
| Batch Q | 1.23 |
| Batch S | 7.44 |
| Batch T | 15.9 |
| Batch U | −23.41 |
| Batch V | −51.44 |
| Batch W | −3.93 |
| Batch X | −43.33 |
| Batch Y | −38.33 |
| Batch Z | 2.33 |
| Batch S20 | −44.51 |

The pharmacological activity of each tested batch is additionally presented in FIG. 3B and Table 2 as values normalized to the SMAD7/p-actin ratio of Batch A (positive control). A performance score is given to each tested batch:

$$Performance Score = - [ ( \frac{SMAD 7 / β - actin ratio of a test batch}{SMAD 7 / β - actin ratio of Batch A} - 1 ) \times 100 \% ] .$$

In certain embodiments, a Performance Score greater than or equal to −20% indicates good in vitro activity; a Performance Score between −20% to −40% indicates moderate in vitro activity; a Performance Score lower than or equal to −40% indicates poor in vitro activity.

TABLE 2
Mean Performance Score
of SMAD7 ASO batches

| | Performance |
|---|---|
| Batch | Score (%) |
| Negative | −120 |
| Control | |
| Batch B | −18.3 |
| Batch C | −56.9 |
| Batch D | −57.7 |
| Batch E | −20.1 |
| Batch F | −29.8 |
| Batch G | −117 |
| Batch H | −24.2 |
| Batch I | −55.3 |
| Batch J | 11.79 |
| Batch K | −107 |
| Batch L | −159 |
| Batch M | −35.7 |
| Batch N | −28.3 |
| Batch O | −13 |
| Batch P | −62.4 |
| Batch Q | −139 |
| Batch S | −117 |
| Batch T | −145 |
| Batch U | −56.6 |
| Batch V | −14 |
| Batch W | −92.6 |
| Batch X | −12.3 |
| Batch Y | −42.7 |
| Batch Z | −139 |
| Batch S20 | −11.4 |

These results suggest that the 26 different tested batches of SMAD7 ASO exhibit significantly different abilities to down-regulate SMAD7 protein expression: Batches B, J, O, V, X, and S20 have good in vitro activity; Batches E, F, H, M, N, have moderate in vitro activity; Batches C, D, G, I, K, L, P, Q, S, T, U, W, Y, Z have poor in vitro activity.

For Batches A, X, K, and T, Real-Time PCR (RT-PCR) was also performed to evaluate their effect on SMAD7 mRNA expression. After transfection, cells were harvested and RNA was extracted using a PureLink mRNA mini kit (Thermo Fisher Scientific) according to the manufacturer's instructions. A constant amount of RNA (1 μg/sample) was reverse-transcribed into complementary DNA (cDNA), and 1 μL cDNA/sample was then amplified by RT-PCR using iQ SYBR Green Supermix (Bio-Rad Laboratories) and the following β-actin primers: FWD: 5′-AAGATGACCCAGATCATGTTTGAGACC-3′; REV: 5′-AGCCAGTCCAGACGCAGGAT-3′. SMAD7 RNA expression was evaluated using a Tagman assay (Life Technologies) and calculated relative to the β-actin gene on the base of the ΔΔCt algorithm. Values derived from all the observations were expressed as mean±SEM of at least three independent experiments (FIG. 3C). Data were analyzed using one-way analysis of variance (ANOVA) followed by Dunnett's Multiple Comparison Test. Significance was defined as P-values<0.05. These results suggest that the different SMAD7 ASO batches exhibit different abilities to down-regulate SMAD7 mRNA expression.

### Example 3: Clinical Activity of SMAD7 Antisense Oligonucleotide Diastereomeric Mixtures

Different batches of SMAD7 ASO (Batches A, H, K, P, and T, V, X) were tested in Crohn's disease patients and assessed for their clinical efficacy.

In the clinical studies, SMAD7 ASO was formulated as a gastro-resistant delayed release tablet, designed to release the SMAD7 ASO in the intestine via a pH-dependent mechanism. Clinical data from patients receiving SMAD7 ASO tablets once daily at a dose of 160 mg/day in four clinical trials, referred to hereafter as CT-A, CT-B, CT-E, and CT-P, were obtained and evaluated. In the CT-A, CT-E, and CT-P studies, Group 2, 3, 4, 5, or 6 patients were treated for a total of 12 consecutive weeks. In the CT-B study, Group 1 patients were treated for 14 consecutive days.

For data analysis, patients were grouped as follows:

Group 1: patients treated with tablets manufactured from Batch A;
Group 2: patients treated with tablets manufactured from Batch X;
Group 3: patients treated with tablets manufactured from Batch V;
Group 4: patients treated with tablets manufactured using Batch P and Batch T;
Group 5: patients treated with tablets manufactured using Batch H and Batch K; and
Group 6: patients treated with tablets manufactured using Batch K and Batch P.

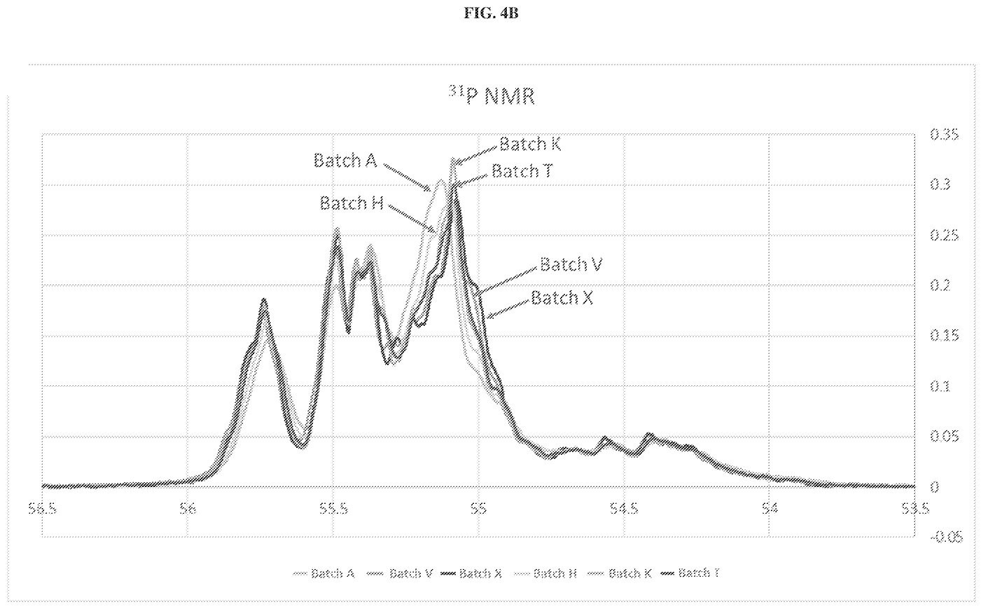

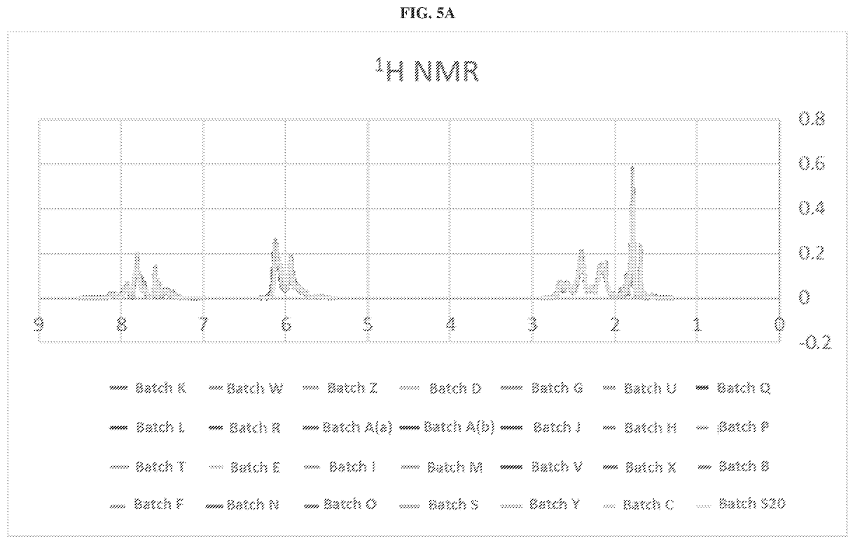

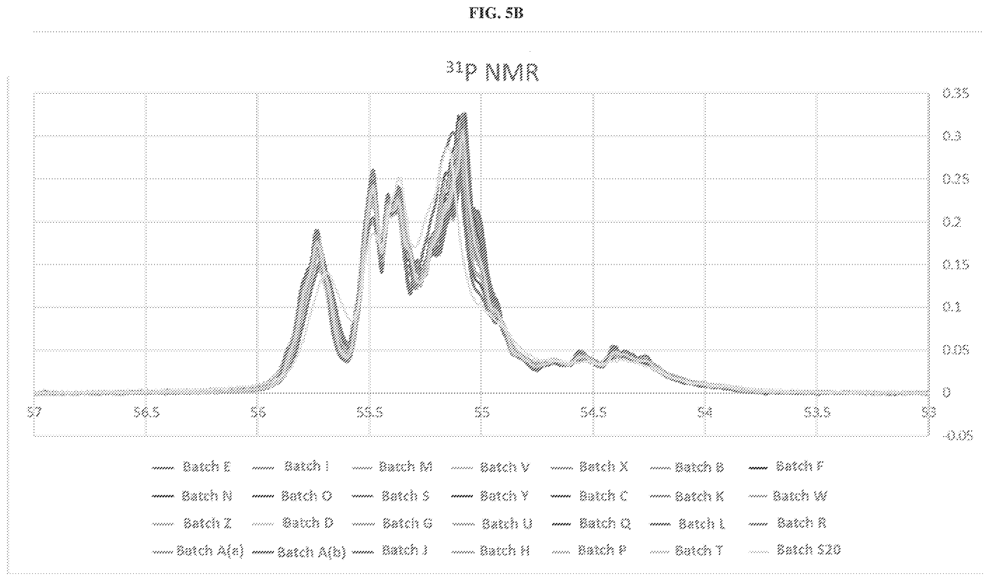

The variation in Crohn's disease activity index (CDAI) was evaluated for each patient at baseline and at the end of week 4 of the treatment period. The mean change in CDAI score between week 4 and baseline (“ΔCDAI”) was calculated for each patient group and reported as shown in FIG. 4A. Clinical response (≥70-point CDAI reduction) was observed in patients treated with tablets manufactured from Batches A, X, and V, with patients treated with tablets manufactured from Batches A and X showing 100-point clinical response (≥100-point CDAI reduction). Patients treated with tablets manufactured using Batches P/T, H/K, and K/P did not show clinically significant improvements. The results suggest that different batches of SMAD7 ASO present different pharmacological efficacies.

### Example 4: Nuclear Magnetic Resonance Spectroscopy of SMAD7 Antisense Oligonucleotide Diastereomeric Mixtures

High resolution Phosphorus-31 nuclear magnetic resonance (³¹P-NMR) measurements (1-D spectrum and longitudinal relaxation measurements) at 14.1 T (in solution) were used to map the local structure at each phosphor atom and to provide diastereomeric profiles of the SMAD7 ASO batches.

Samples corresponding to Batches A-Z and S20 were analysed, each in duplicate. Briefly, the samples were dissolved in D₂O at a concentration of about 9 mg/0.5 mL and immediately measured on an Agilent Inova 600 MHz spectrometer (14.1 Tesla) at 25.0° C. to obtain ¹H- and ³¹P-NMR spectra following standard protocols. For the ¹H-NMR experiment, 128 transients were accumulated (FIG. 5A); for the ³¹P-NMR experiment. 2000 transients were accumulated with 2 s relaxation time. The ¹H DOSY-NMR spectra were assessed to confirm that the samples were not contaminated with small molecules (e.g., solvents or additives) or with by-products, and, therefore, differences observed in the ³¹P-NMR spectra may be ascribed to the diastereomeric profiles of the samples. The ³¹P-NMR spectra are shown in FIGS. 5B-5FF. The spectral lineshapes for the two duplicate specimens of each sample (i.e., batch) match each other, but there are considerable differences in the lineshapes between batches. A magnified analysis of the high resolution ³¹P-NMR spectra (FIGS. 5C-5CC) reveals that the spectra of different samples have remarkable differences in lineshapes (intensity, frequency, phase, and linewidth) in the range between 54.8-55.5 ppm concerning both signal structures and relative intensities. In contrast to a Lorentzian lineshape characteristic of a single species, the resonance in the range between 54.8-55.5 ppm is a convolution of a multiplicity of signals, due to the co-existence of a manifold of diastereomers. On the other hand, the 55.5-56 ppm region of the ³¹P-NMR spectra appeared to have a more conserved lineshape among batches.

These observations demonstrate that different SMAD7 ASO batches have differing diastereomeric profiles and that ³¹P-NMR is suitable for fingerprinting the stereochemistry of antisense oligonucleotides, such as SMAD7 ASOs. Particularly, the 54.8-55.5 ppm region is hypersensitive to differences in the diasteromeric composition of a given SMAD7 ASO sample.

### Example 5: Circular Dichroism Spectroscopy of SMAD7 Antisense Oligonucleotide Diastereomeric Mixtures

Circular dichroism, particularly variable-temperature circular dichroism (VTCD), was employed to assess the overall stereochemistry of the various SMAD7 ASO batches.

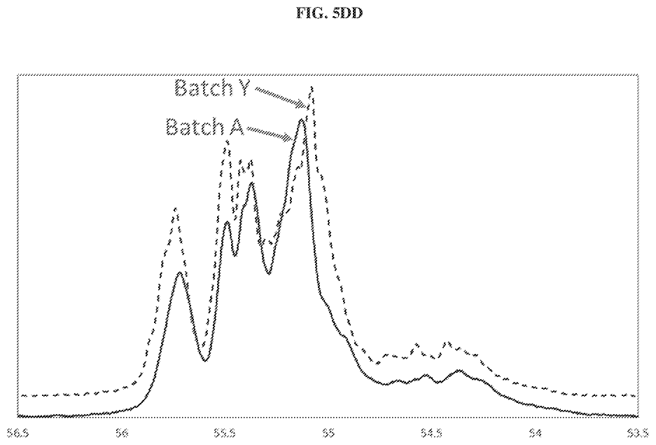

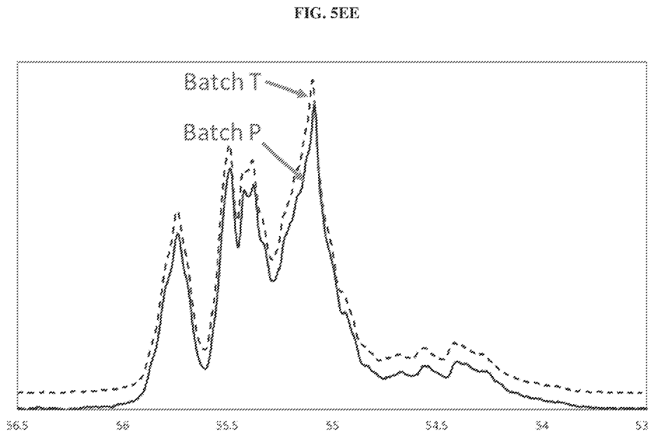

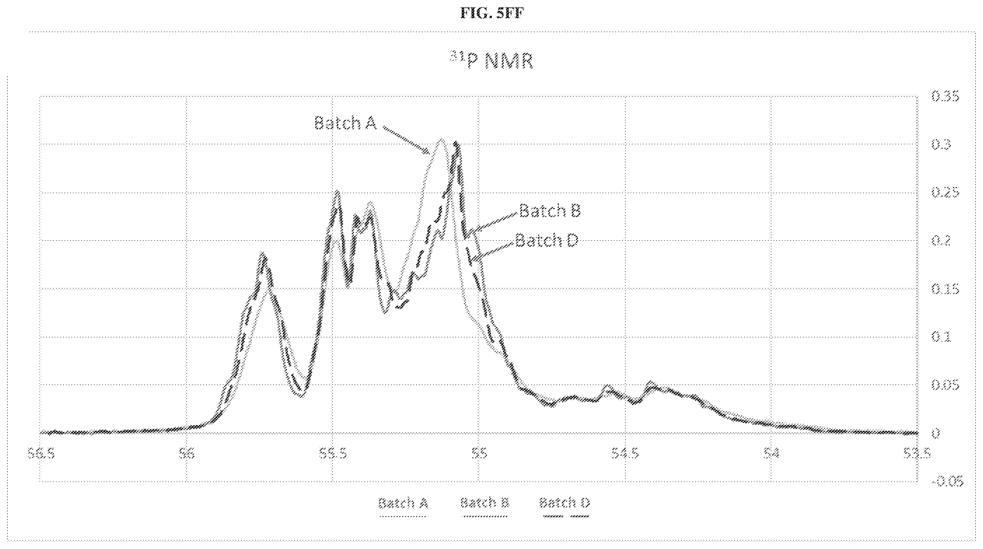

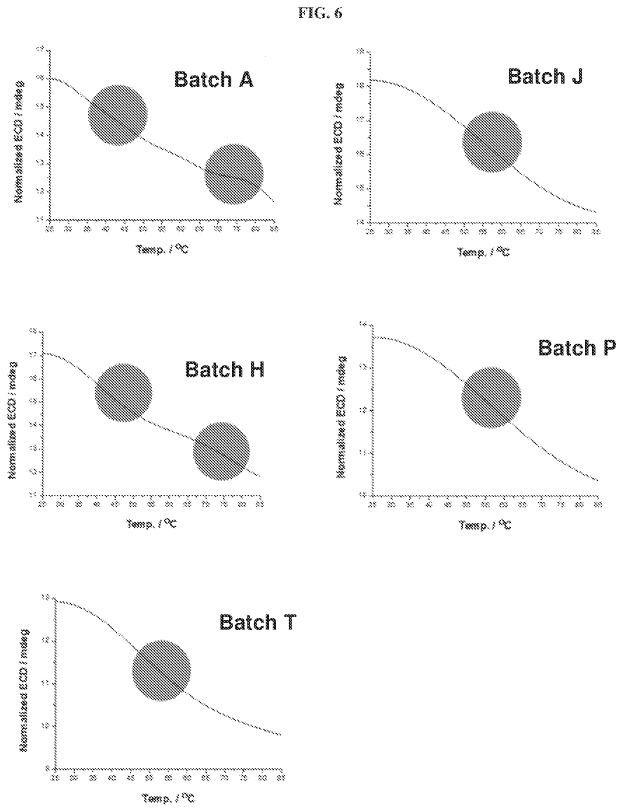

Five samples corresponding to Batches A, J. H. P. and T were analysed, each in duplicate. The VTCD spectra are shown in FIG. 6. Batch A and Batch H both have an inflection point at about 45° C. Batches J, P. and T, all have an inflection point around 50° C.±5° C. Batch H has multiple inflection points. The VTCD spectra of Batch A and Batch H, although different from each other, further suggest a more complex set of transitions of the samples (2 inflection points) as compared to the other samples.

### Example 6: Chemometric Modeling of Spectroscopic Data of Oligonucleotide Diastereomeric Mixtures

Chemometric modeling is used to relate the spectroscopic data of oligonucleotide diastereomeric mixtures (e.g., diastereomeric mixtures of SMAD7 ASO) to pharmacological and/or clinical efficacy.

Spectroscopic data, such as ¹H-NMR, ³¹P-NMR, and VTCD spectra, are classified by means of pattern recognition using Singular Value decomposition (SVD) Principal Component Analysis (PCA). A vector is built using ³¹P-NMR intensities as a function of resonance (about 200 datapoints for each sample), which is thereafter normalized to total integral, in order to obtain a concentration-independent vector. The vectors for different oligonucleotide preparations are then subjected to PCA at various ppm bands, for example in the area around 55 ppm, and more specifically, for example, the areas between 54.8-55.5 ppm. Similar ³¹P-NMR spectra correspond to similar structural manifolds. Subjecting the vectors to PCA (e.g., 54.8-55.5 ppm areas) reveals stereochemical similarities and diversities which can correspond to the presence or absence of pharmacological efficacy and clinical activity.

### Example 7: Principal Component Analysis of Phosphorus-31 Nuclear Magnetic Resonance Data of SMAD7 Oligonucleotide Diastereomeric Mixtures

To quantitatively attribute the variations in the ³¹P-NMR spectra of different SMAD7 ASO batches to their diastereomeric profiles and to associate such variations with pharmacological/clinical efficacy, the ³¹P-NMR spectra were subject to principal components analysis (PCA).

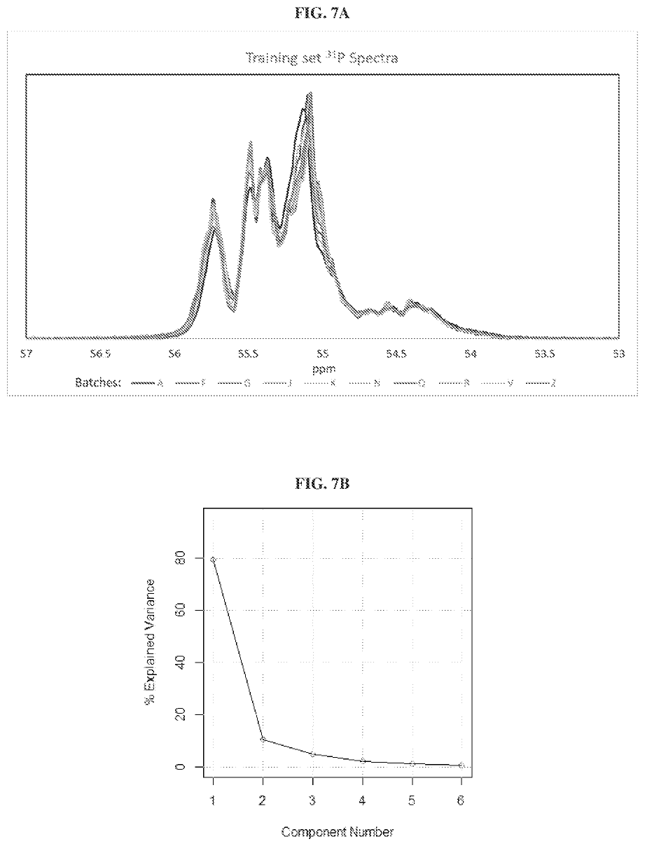

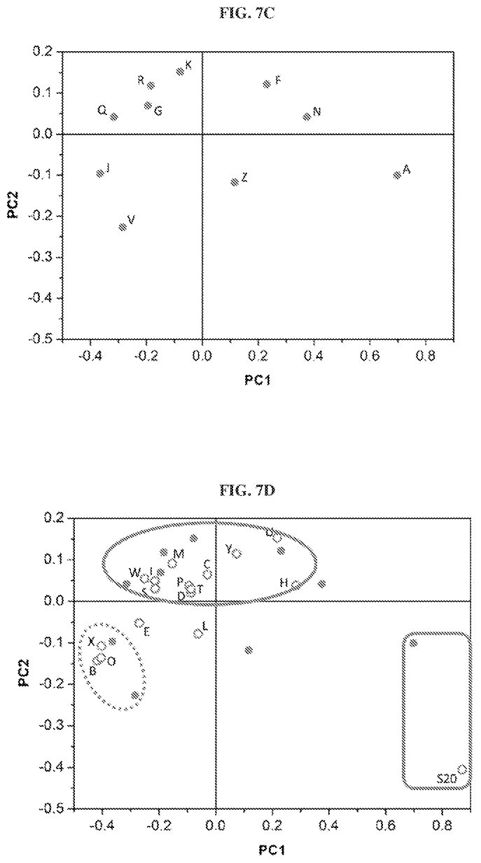

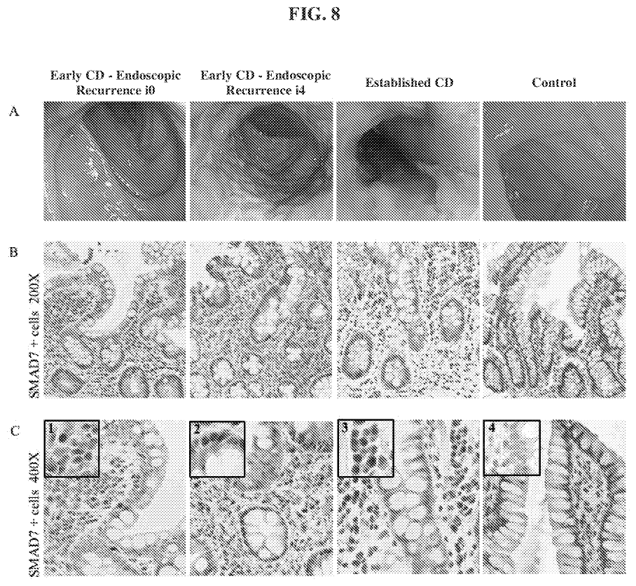

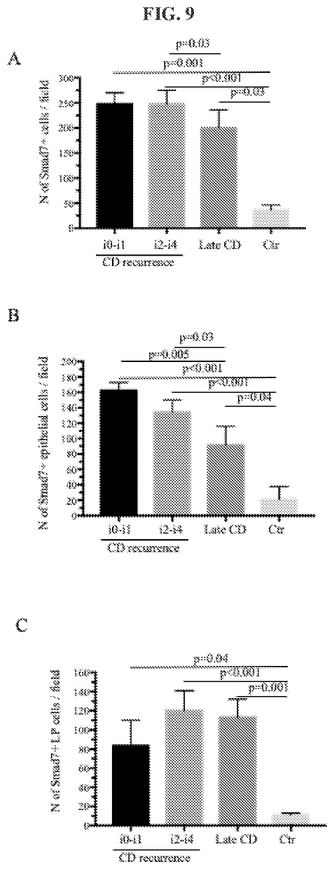

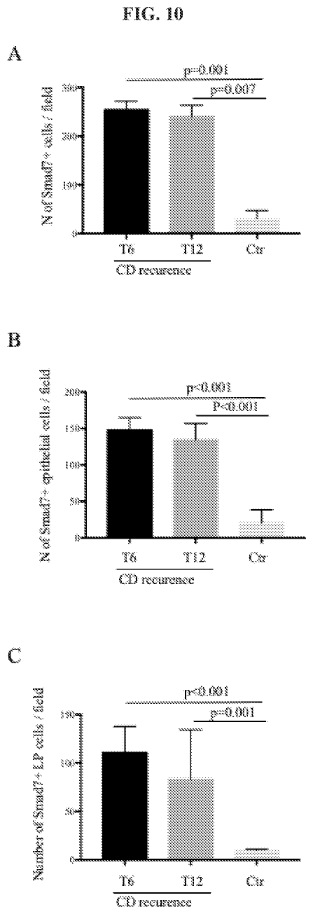

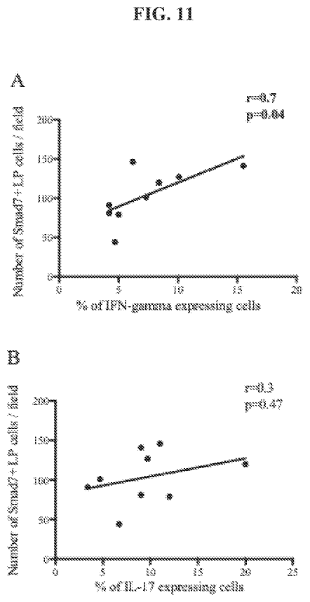

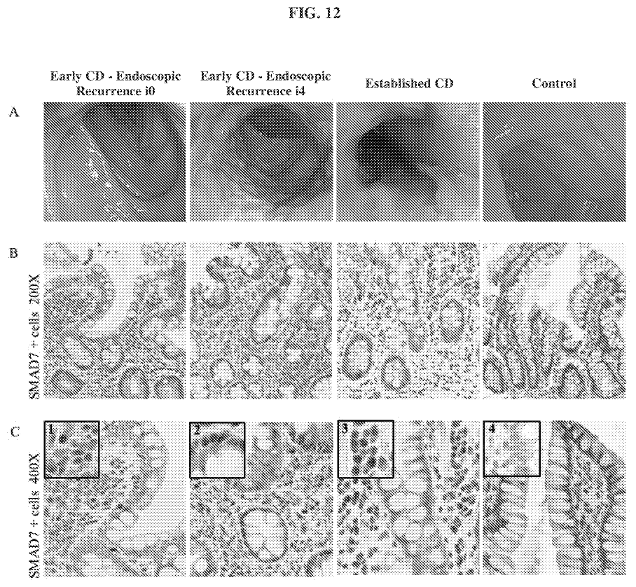

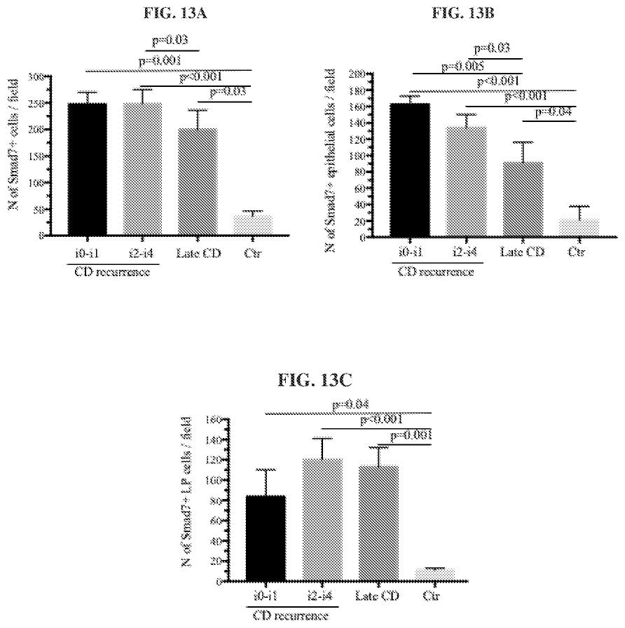

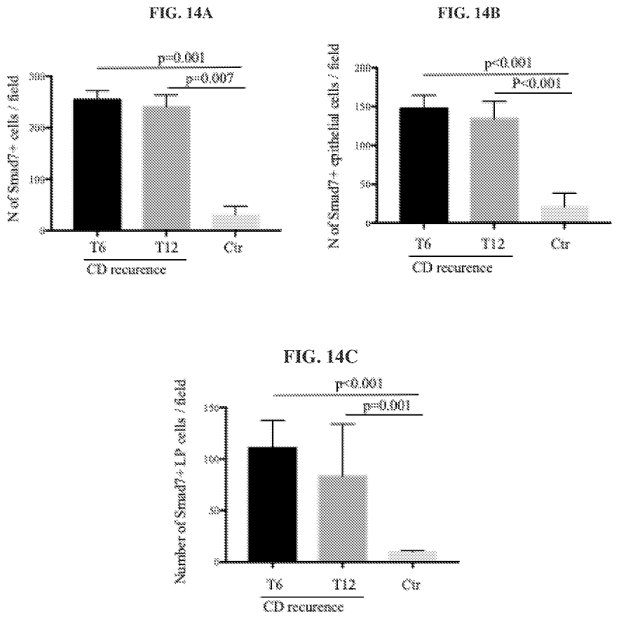

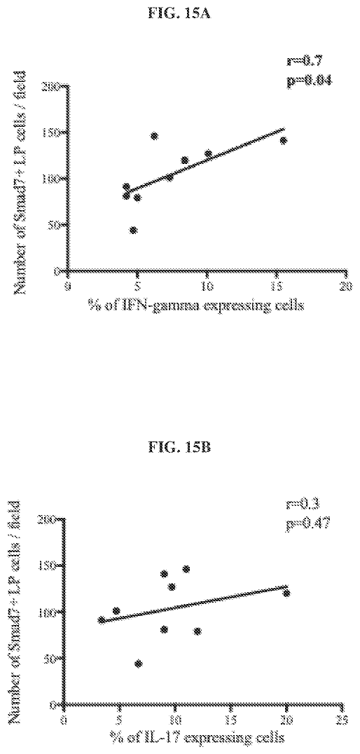

Briefly, the ³¹P-NMR spectra of samples corresponding to Batches A-Z and S20 (as described in Example 4) were apodized (LB=4 Hz), phase and baseline corrected using MestReNova software and normalized by scaling the intensity to the area (integral) between 53 and 57 ppm. The resulting spectra data were exported as an Excel matrix. The principal components analysis was performed using the Chemometrics Agile Tool (CAT) software (www.gruppochemiometria.it/index.php/software/19-download-the-r-based-chemometric-software). The ³¹P-NMR spectra of Batches A, F, G, J, K, N, Q, R, V, and Z (FIG. 7A) were input as the “training set” to determine the number of principal components (PCs) for retention and to generate the scree plot shown in FIG. 7B. The scree plot shows that the first two PCs explain 90% of the variance in the data, whereas the first six PCs explain 99%. Accordingly, the first two components (PC1 and PC2) were retained for analysis and to build the PCA plot of the training set spectra (FIG. 7C). ³¹P-NMR spectra of samples from additional 17 batches were then projected into the PCA plot generated with the training set spectra (FIG. 7D).

The PCA plot per ³¹P-NMR spectrum (FIG. 7D) was analysed in view of the in vitro activity of each sample (as described in Example 2) and revealed that the spectra can be grouped into three clusters. Cluster 1 centres round (0, 0), represented by a solid oval; cluster 2 centres around (−0.35, −0.2), represented by a dotted oval; cluster 3 is in the area of PC1>0.7, represented by a solid rectangle. Samples that fall within clusters 2 and 3 show good in vitro activity; samples that fall within cluster 1 show poor in vitro activity. The superimposition of ³¹P-NMR spectra of three batches A, B, and D, falling in cluster 3, cluster 2, and cluster 1, respectively, in the PCA score plot (FIG. 7D), is presented in FIG. 5FF.

Projection of the ³¹P-NMR spectra on PC1/PC2 plane, and evaluation of the in vitro (IV) pharmacology and a clinical efficacy (CDAI plot) revealed a correlation between IV pharmacology and NMR PC score versus clinical performance. As shown in FIG. 4A and FIG. 4B, representative batches A, H, K, P, T, V, and X, whose ³¹P-NMR spectra are shown in FIG. 4B, displayed a different clinical performance (see CDAI plot in FIG. 4A). Batch A, within cluster 3 in the PCA plot, and Batches V and X, within cluster 2 of the PCA plot, are identified as clinically efficacious; Batches H, K, P, and T, within cluster 2 in the PCA plot, do not have significant clinical efficacy (see Example 3).

### Example 8: SMAD7 Expression in Postoperative Crohn's Patients

### Example 8A: Patient Selection

Patients were selected for assessing the expression of SMAD7 in Crohn's disease (CD) samples of different disease phases.

17 CD patients previously undergone ileocolonic resection were included in the study as early CD samples. The demographic and clinical characteristics of these patients are shown in Table 3. Ileocolonoscopy was performed with biopsies collected from the neoterminal ileum (10-20 cm above the anastomosis) of the patients to assess the endoscopic recurrence of CD 6 and/or 12 months after surgery (FIG. 12). At the time of ileocolonoscopy, 82% of the patients were receiving mesalamine and 12% were receiving thiopurines. Endoscopic recurrence was graded according to the Rutgeerts's score.

11 of the early CD patients underwent ileocolonoscopy 6 months after ileocolonic resection. 7 (63.6%) of the 11 patients showed endoscopic recurrence, of which 4 also had clinically active disease (CD activity index (CDAI)>150). The remaining 4 (36.4%) had no endoscopic lesion, of which 2 had underwent a second endoscopy 12 months after the resection as they became symptomatic. The remaining 6 early CD patients underwent endoscopy 12 months after the intestinal resection.

TABLE 3
Demographic and Clinical
Characteristics of Patients

| Patients Characteristics | N = 17 |
|---|---|
| Age | |
| Years, Median (range) | 37 (21-64) |
| Sex, | |
| Male, N (%) | 14 (82%) |
| Smoking habits, N (%) | |
| Yes | 1 (6%) |
| No | 16 (94%) |
| CD duration | |
| Months, Median (range) | 180 (12-320) |
| Age at diagnosis, N (%) | |
| A1: ≤16 years | 1 (6%) |
| A2: 17-40 years | 13 (76%) |
| A3: over 40 years | 3 (18%) |
| CD behaviour, N (%) | |
| B1: inflammatory | 0 |
| B2: Stricture | 11 (65%) |
| B3: Penetrating | 6 (35%) |
| CD location, N (%) | |
| L1: Ileal | 17 (100%) |
| L2: Colonic | 0 |
| L3: Ileocolonic | 0 |
| Harvey Bradshaw index | |
| at time of endoscopy | |
| Median (range) | 4 (2-9) |
| Remission | 13 (76%) |
| Active | 4 (24%) |
| CD medication at time | |
| of endoscopy | |
| No medication | 1 (6%) |
| 5-aminosalicylic acid | 14 (82%) |
| Corticosteroids | 0 |
| Thiopurine alone | 2 (12%) |
| TNFs alone | 0 |

For established CD samples, mucosal samples were collected from the resected ileum of 11 CD patients (8 male; median age 53 (21-69) years; median disease duration 149 (36-312) months) at the time of ileoc:olonic resection. All 11 patients had lesions confined to the terminal ileum and received surgical treatment due to the chronically active disease poorly responsive to medical treatment. At the time of surgery, 9 patients were on steroids, 2 of which were simultaneously receiving azathioprine, and the remaining 2 patients had previously been treated with anti TNF-α antibody.

As the control group, ileal biopsies were taken from 5 patients who underwent ileocolonoscopy for irritable bowel syndrome. No endoscopic lesion was found in these patients and the ileal mucosa was histologically normal.

Tissue sections obtained from the above-described patients were preserved by formalin fixation followed by paraffin embedding until further use.

### Example 8B: Assessment of SMAD7 Expression in Crohn's Disease Patients

Immunohistochemistry was performed to assess the expression of SMAD7 in ileal mucosa of in CD patients of different disease phases—early CD patients (i.e., postoperative CD patients with or without endoscopic recurrence) and established CD patients.

All reagents were from Sigma-Aldrich (Milan, Italy) unless specified. Formalin-fixed, paraffin-embedded sections as described in Example 8A were deparaffinized and dehydrated through xylene and ethanol. The antigen retrieval was performed in citrate buffer (pH 6.0) for 20 minutes in microwave. Immunohistochemical staining was performed using a rabbit anti-SMAD7 primary antibody (orb11386; Biorbyt Ltd, Cambridge. UK), at room temperature for 1 hour. Immunoreactive cells were detected using MACH4 Universal HRP-Polymer (Biocare Medical, Concord, Calif., USA) in combination with 3,3′-Diaminobenzidine (DAB) (Dako North America, Carpinteria, Calif., USA) chromogen and hematoxylin as counterstain, according to the manufacturer's protocols. Sections stained using an isotype IgG antibody (R&D Systems. Minneapolis, Minn., USA) as the primary antibody were used as control. Stained sections were examined with LEICA DM14000 B microscope, and the SMAD7-expressing cells were counted in 5 high-power fields per microscope slide of section.

Statistical differences were assessed with the GraphPad Prism statistical PC program (GraphPad Software, San Diego, Calif.). Nonparametric data were analyzed using the Mann-Whitney U-test for comparison between two groups or Kruskal-Wallis test for multiple comparison. Significance of correlation was determined using the Spearman non-parametric correlation. A P value of less than 0.05 was considered statistically significant.

Exemplary results are shown in FIG. 12 and FIG. 13. SMAD7-positive cells were more evident in CD patient ileal mucosa than in the control group (FIGS. 12B and 12C) under immunohistochemical staining. Particularly, SMAD7 accumulated in the cytoplasm and nucleus of both epithelial cells and lamina propria mononuclear cells (LPMCs) (FIG. 12C). Accordingly, the numbers of SMAD7-positive cells were significantly higher in samples taken from the neoterminal ileum of early CD patients (i.e., postoperative CD patients without endoscopic recurrence (i0) or with endoscopic recurrence (i4)) and established CD patients than those from patients of the control group (FIG. 13). Furthermore, the numbers of SMAD7-positive cells were higher in samples from early CD patients than in samples from late CD patients in the whole intestinal mucosa (FIG. 13A) and in the epithelial compartment (FIG. 13B).

FIG. 14 presents the analysis of ileal mucosa of postoperative CD patients at 2 different time points (i.e., 6 and 12 months after ileocolonic resection), which shows no significant difference in the number of SMAD7-expressing cells.

The data suggest that the expression of SMAD7 in mucosa of CD patients is enhanced at the early disease phase. e.g., a short period following the intestinal resection, and maintained at high level during the course of the disease and/or disease recurrence. The results further suggest that SMAD7 upregulation may be a suitable marker of CD recurrence and that modulating SMAD7 activity (e.g., inhibiting SMAD7) may be beneficial in the prevention and/or treatment of postoperative recurrence of CD.

### Example 8C: Correlation Between SMAD7 Expression and Cytokine Expression

To assess the correlation between SMAD7 expression with the number of cytokine-secreting cells, fluorescence-activated cell sorting (FACS) analysis was used.

Lamina propria mononuclear cells (LPMC) were isolated from ileal biopsies samples and intestinal resection specimens from patients selected as described in Example 8A and suspended in RPMI 1640 medium, supplemented with 10% inactivated fetal bovine serum (FBS), penicillin (P) (100 U/mL), and streptomycin (S) (100 μg/mL) (Life Technologies-GibcoCRL, Milan, Italy).

For flow cytometry analysis, LPMCs were seeded in 96-well U-bottom culture dishes and stimulated with PMA (10 ng/mL), ionomycin (1 μg/mL), and brefeldinA (10 μg/mL; eBioscience, San Diego, Calif.). After 5 hours, cells were stained with anti-CD3-PerCP (1:50 dilution; BD Biosciences, San Jose, Calif.) and fixed with 1% formaldehyde for 20 minutes. Subsequently, cells were permeabilized with 0.5% saponin in 1% bovine serum albumin (BSA) FACS buffer and stained with the following antibodies: anti-IFN-α-PE (1:50 dilution; BD Biosciences), anti-IL-17A-APC (1:50 dilution; eBioscience). Appropriate isotype-matched controls (BD Biosciences) were included in all experiments. Cells were analyzed using a FACSCalibur cytometer and Cell-QuestPro software. Statistical differences were assessed as described in Example 8B.

Exemplary results are shown in FIG. 15. A significant correlation was observed between the number of SMAD7-expressing cells and the number of IFN-γ-secreting cells. No significant correlation was observed between the number of SMAD7-expressing cells with the number of IL-17A-producing cells.

### EQUIVALENTS

The disclosure can be embodied in other specific forms with departing from the essential characteristics thereof. The foregoing embodiments therefore are to be considered illustrative rather than limiting on the disclosure described herein. The scope of the disclosure is indicated by the appended claims rather than by the foregoing description, and all changes that come within the meaning and range of equivalency of the claims are intended to be embraced therein.

What is claimed is:

1. A pharmaceutical composition, comprising a plurality of diastereomers of a SMAD7 antisense oligonucleotide (ASO) having a sequence according to SEQ ID NO: 1 (5′-GTXGCCCCTTCTCCCXGCAGC-3′) in which all internucleoside linkages are O,O-linked phosphorothioate linkages and X is 5-methyl 2′-deoxycytidine, wherein the plurality of diastereomers has a phosphorus-31 nuclear magnetic resonance (³¹P-NMR) spectrum comprising:
a) one or more resonances between 54.8 to 55.5 ppm; and
b) a first principal component 1 (PC1) score and a second principal component 2 (PC2) score, wherein:
(i) the PC1 score is below about −0.20 or above about 0.25 and the PC2 score is below about 0.00; or
(ii) the PC1 score is outside of the range of −0.32 to 0.30 and/or the PC2 score that is outside the range of 0.00 to 0.20.

2. The pharmaceutical composition of claim 1, wherein the ³¹P-NMR spectrum comprises a PC1 score that is below about −0.20 and a PC2 score that is below about 0.00.

3. The pharmaceutical composition of claim 1 or 2, wherein the ³¹P-NMR spectrum comprises a PC1 score that is from about −0.47 to about −0.20 and a PC2 score that is from about −0.27 to about 0.00.

4. The pharmaceutical composition of claim 1, wherein the ³¹P-NMR spectrum comprises a PC1 score that is above about 0.25 and a PC2 score that is below about 0.00.

5. The pharmaceutical composition of claim 1 or 4, wherein the ³¹P-NMR spectrum comprises a PC1 score that is from about 0.65 to about 0.9 and a PC2 score that is from about −0.47 to about 0.00.

6. The pharmaceutical composition of claim 1, wherein the ³¹P-NMR spectrum comprises a PC1 score that is outside of the range of −0.32 to 0.30 when the PC2 score is above 0.00 and below 0.20.

7. The pharmaceutical composition of claim 1 or 6, wherein the ³¹P-NMR spectrum comprises a PC2 score is outside the range of 0.00 to 0.20 when the PC1 is above −0.32 and below 0.30.

8. The pharmaceutical composition of any one of claims 1-7, wherein the ³¹P-NMR spectrum comprises two or more resonances between 54.8 to 55.5 ppm.

9. The pharmaceutical composition of claim 8, wherein the two or more resonances between 54.8 to 55.5 ppm have different intensities.

10. The pharmaceutical composition of any one of claims 1-9, wherein the ³¹P-NMR spectrum comprises, independently at about 54.8 ppm, at about 54.9 ppm, at about 55.0 ppm, at about 55.1 ppm, at about 55.2 ppm, at about 55.3 ppm, at about 55.4 ppm, and/or at about 55.5 ppm, one or more resonances.

11. The pharmaceutical composition of any one of claims 1-10, wherein the ³¹P-NMR spectrum comprises, independently at about 54.8 ppm, at about 54.9 ppm, at about 55.0 ppm, at about 55.1 ppm, at about 55.2 ppm, at about 55.3 ppm, at about 55.4 ppm, and/or at about 55.5 ppm, two or more resonances.

12. The pharmaceutical composition of any one of claims 1-11, comprising 100 millimoles to 5 moles of SMAD7 ASO.

13. The pharmaceutical composition of any one of claims 1-12, comprising 100 millimoles to 2 moles, 100 millimoles to 900 millimoles, 300 millimoles to 5 moles, 300 millimoles to 2 moles, 300 millimoles to 900 millimoles, 900 millimoles to 5 moles, or 900 millimoles to 2 moles of SMAD7 ASO.

14. The pharmaceutical composition of any one of claims 1-13, comprising about 300 millimoles of SMAD7 ASO.

15. The pharmaceutical composition of any one of claims 1-13, comprising about 900 millimoles of SMAD7 ASO.

16. The pharmaceutical composition of any one of claims 1-13, comprising about 2 moles of SMAD7 ASO.

17. The pharmaceutical composition of any one of claims 1-13, comprising greater than 300 millimoles, greater than 900 millimoles, or greater than 2 moles of SMAD7 ASO.

18. The pharmaceutical composition of any one of claims 1-17, comprising:
a) about 0.5% to about 30% by weight of the SMAD7 ASO;
b) about 20% to about 50% by weight mannitol;
c) about 10% to about 30% by weight microcrystalline cellulose; and
d) an enteric coating comprising an ethylacrylate-methacrylic acid copolymer.

19. The pharmaceutical composition of any one of claims 1-18, comprising:
a) an intra-granular phase comprising:
i) about 5% to about 10% by weight of the SMAD7 ASO;
ii) about 40% by weight mannitol;
iii) about 8% by weight microcrystalline cellulose;
iv) about 5% by weight hydroxypropyl methylcellulose; and
v) about 2% by weight sodium starch glycolate;
b) an extra-granular phase comprising:
i) about 17% by weight microcrystalline cellulose;
ii) about 2% by weight sodium starch glycolate,
iii) about 0.4% by weight magnesium stearate; and
c) an enteric coating comprising an ethylacrylate-methacrylic acid copolymer,
wherein the percentages by weight are the weights of the ingredients compared to the total weight of the pharmaceutical composition.

20. The pharmaceutical composition of any one of claims 1-18, comprising:
a) about 5% to about 30% by weight of the SMAD7 ASO;
b) about 20% to about 50% by weight mannitol;
c) about 10% to about 30% by weight microcrystalline cellulose,
d) about 0.5% to about 10% by weight hydroxypropyl methylcellulose;
e) about 0.5% to about 10% by weight sodium starch glycolate;
f) about 0.05% to about 1% by weight magnesium stearate;
g) about 0.5% to about 10% by weight Opadry® AMB; and
h) about 5% to about 20% by weight Acryl-EZE®.

21. The pharmaceutical composition of any one of claims 1-18, comprising:
a) about 8.5% by weight of the SMAD7 ASO;
b) about 40% by weight mannitol;
c) about 25% by weight microcrystalline cellulose,
d) about 5% by weight hydroxypropyl methylcellulose;
C) about 4% by weight sodium starch glycolate;
f) about 0.4% by weight magnesium stearate;
g) about 4% by weight Opadry® AMB; and
h) about 10% to about 15% by weight Acryl-EZE®.

22. The pharmaceutical composition of any one of claims 1-18, comprising:
a) about 23% by weight of the SMAD7 ASO;
b) about 28% by weight mannitol;
c) about 25% by weight microcrystalline cellulose,
d) about 5% by weight hydroxypropyl methylcellulose;
e) about 4% by weight sodium starch glycolate;
f) about 0.4% by weight magnesium stearate;
g) about 4% by weight Opadry® AMB; and
h) about 7% to about 12% by weight Acryl-EZE®.

23. The pharmaceutical composition of any one of claims 1-22, wherein the pharmaceutical composition is formulated as a tablet.

24. The pharmaceutical composition of any one of claims 1-23, wherein the pharmaceutical composition down-regulates SMAD7 mRNA and/or protein in a cell.

25. The pharmaceutical composition of claim 24, wherein the down-regulation of SMAD7 protein expression is more than 40% compared to an untreated cell.

26. The pharmaceutical composition of any one of claims 1-25, wherein the pharmaceutical composition is clinically efficacious in treating or preventing inflammatory bowel disease.

27. The pharmaceutical composition of claim 26, wherein the inflammatory bowel disease is Crohn's disease.

28. The pharmaceutical composition of claim 27, wherein the Crohn's disease is postoperative recurrence of Crohn's disease.

29. The pharmaceutical composition of any one of claims 1-28 for oral administration.

30. The pharmaceutical composition of any one of claims 1-29 for oral administration, wherein the SMAD7 ASO is administered to a subject in need thereof at a dose of about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg.

31. The pharmaceutical composition of any one of claims 1-30 for oral administration about every 6 hours, about every 12 hours, about every 24 hours, about every 48 hours, about every 72 hours, every day, two-times a week, once in 2 weeks, or once a month.

32. The pharmaceutical composition of any one of claims 1-31 for oral administration at a dose of about 160 mg daily.

33. A method of treating or preventing an inflammatory bowel disease in a subject in need thereof, comprising administering to the subject the pharmaceutical composition of any one of claims 1-32.

34. Use of a pharmaceutical composition of any one of claims 1-32 for the manufacture of a medicament for the treatment of an inflammatory bowel disease.

35. A pharmaceutical composition of any one of claims 1-32 for use in the treatment of an inflammatory bowel disease.

36. The method of claim 33, use of claim 34, or pharmaceutical composition for use of claim 35, wherein the inflammatory bowel disease is Crohn's disease.

37. The method of claim 33 or 36, use of claim 34 or 36, or pharmaceutical composition for use of claim 35 or 36, wherein the Crohn's disease is postoperative recurrence of Crohn's disease.

38. A method of preventing or treating postoperative recurrence of Crohn's disease (CD) in a subject in need thereof, comprising inhibiting SMAD7 in the subject.

39. The method of claim 38, wherein at least one sample from the subject has an elevated SMAD7 level relative to a known control level, wherein the known control level is the SMAD7 level in a sample collected from the subject prior to or during the surgical treatment for CD or the SMAD7 level in a sample collected from a healthy subject without CD.

40. A method of preventing or treating postoperative recurrence of Crohn's disease (CD) in a subject in need thereof, comprising preventing a Crohn's Disease Activity Index (CDAI) score greater than 200 of the subject or reducing CDAI score for at least 50 points.

41. The method of claim 40, comprising preventing a CDAI score greater than 150.

42. The method of claim 40, comprising reducing CDAI score for at least 100 points.

43. A method for predicting postoperative recurrence of Crohn's disease (CD) in a subject in need thereof, comprising determining the level of SMAD7 in a first sample from the subject, wherein an elevated SMAD7 level relative to a known control level is predictive of the recurrence of CD, wherein the known control level is the SMAD7 level in a sample collected from the subject prior to or during the surgical treatment for CD or the SMAD7 level in a sample collected from a healthy subject without CD.

44. A method of identifying a subject at risk of postoperative recurrence of Crohn's disease (CD), comprising: determining the level of SMAD7 in a first sample from the subject, wherein an elevated SMAD7 level relative to a known control level identifies the subject as being at risk for the recurrence of CD, wherein the known control level is the SMAD7 level in a sample collected from the subject prior to or during the surgical treatment for CD or the SMAD7 level in a sample collected from a healthy subject without CD.

45. The method of claim 43 or 44, comprising:
a) if the SMAD7 level is elevated relative to the known control level, then administering to the subject a pharmaceutical composition comprising a SMAD7 ASO; or
b) if the SMAD7 level is not elevated relative to the known control level, then determining the level of SMAD7 in a second sample from the subject.

46. The method of claim 45, wherein the second sample is collected immediately after, about 1 hour after, about 3 hours after, about 6 hours after, about 12 hours after, about 1 day after, about 3 days after, about 1 week after, about 2 weeks after, about 1 month after, about 2 months after, about 3 months after, about 4 months after, about 5 months after, about 6 months after, about 7 months after, about 8 months after, about 9 months after, about 10 months after, about 11 months after, or about 12 months after the first sample.

47. A method of preventing or treating postoperative recurrence of Crohn's disease (CD) in a subject in need thereof, comprising administering to the subject a pharmaceutical composition comprising a SMAD7 ASO.

48. The method of claim 38, wherein inhibiting SMAD7 in the subject comprises administering to the subject a pharmaceutical composition comprising a SMAD7 ASO.

49. The method of claim 47 or 48, wherein the SMAD7 ASO is administered at a dose of about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, or about 200 mg.

50. The method of any one of claims 47-49, wherein the SMAD7 ASO is administered about every 6 hours, about every 12 hours, about every 24 hours, about every 48 hours, about every 72 hours, every day, two-times a week, once in 2 weeks, or once a month.

51. The method of any one of claims 47-50, wherein the SMAD7 ASO is administered at a dose of about 160 mg daily.

52. The method of any one of claims 47-51, wherein the SMAD7 ASO comprises a sequence selected from any one of SEQ ID NOs: 1-6 or a pharmaceutically acceptable salt thereof.

53. The method of any one of claims 47-52, wherein the SMAD7 ASO has a sequence according to SEQ ID NO: 1 (5′-GTXGCCCCTTCTCCCXGCAGC-3′) in which all internucleoside linkages are O,O-linked phosphorothioate linkages and X is 5-methyl 2′-deoxycytidine.

54. The method of any one of claims 47-53, wherein the pharmaceutical composition comprises a plurality of diastereomers of a SMAD7 ASO having a sequence according to SEQ ID NO: 1 (5′-GTXGCCCCTTCTCCCXGCAGC-3′) in which all internucleoside linkages are O,O-linked phosphorothioate linkages and X is 5-methyl 2′-deoxycytidine,
wherein the plurality of diastereomers has a ³¹P-NMR spectrum comprising:
a) one or more resonances between 54.8 to 55.5 ppm; and
b) a first principal component 1 (PC1) score and a second principal component 2 (PC2) score, wherein:
(i) the PC1 score is below about −0.20 or above about 0.25 and the PC2 score is below about 0.00; or
(ii) the PC1 score is outside of the range of −0.32 to 0.30 and/or the PC2 score is outside the range of 0.00 to 0.20.

55. The method of claim 54, wherein the ³¹P-NMR spectrum comprises:
a) a PC1 score below about −0.20 and a PC2 score below about 0.00;
b) a PC1 score above about 0.25 and a PC2 score below about 0.00;
c) a PC1 score from about −0.47 to about −0.20 and a PC2 score from about −0.27 to about 0.00;
d) a PC1 score from about 0.65 to about 0.9 and a PC2 score from about −0.47 to about 0.00;
e) a PC1 score outside of the range of −0.32 to 0.30 when the PC2 score is above 0.00 and below 0.20; and/or
f) a PC2 score outside the range of 0.00 to 0.20 when the PC1 is above −0.32 and below 0.30.

56. The method of any one of claims 38-55, wherein the postoperative recurrence is selected from the group consisting of: endoscopic recurrence, histological recurrence, radiographic recurrence, clinical recurrence, and combinations thereof.

57. The method of any one of claims 38-56, wherein the subject has received at least one surgical treatment for CD.

58. The method of any one of claim 57, wherein the at least one surgical treatment is selected from a group consisting of: bowel resection, ileocolonic resection, colectomy, proctocolectomy, strictureplasty, ileostomy, anal fistulotomy, and combinations thereof.

59. The method of claim 58, wherein the at least one surgical treatment is ileocolonic resection.

60. The method of any one of claims 38-59, wherein the subject exhibits no sign of postoperative recurrence.

61. The method of any one of claims 38-60, wherein the subject exhibits at least one sign of postoperative recurrence.

62. The method of claim 61, wherein the subject exhibits at least one sign of endoscopic recurrence.

63. The method of claim 62, wherein the at least one sign is a mucosal lesion.

64. The method of any one of claims 39-63, wherein the sample is a mucosal sample.

65. The method of claim 39-64, wherein the sample is collected immediately after, about 1 hour after, about 3 hours after, about 6 hours after, about 12 hours after, about 1 day after, about 3 days after, about 1 week after, about 2 weeks after, about 1 month after, about 2 months after, about 3 months after, about 4 months after, about 5 months after, about 6 months after, about 7 months after, about 8 months after, about 9 months after, about 10 months after, about 11 months after, or about 12 months after a surgical treatment for CD.

66. The method of any one of claims 39-65, wherein the SMAD7 level is SMAD7 mRNA level or SMAD7 protein level.

67. The method of any one of claims 39-66, wherein the elevated SMAD7 level is at least about 10%, at least about 20%, at least about 30%, at least about 40%, at least about 60%, or at least about 80% elevated as compared to the known control level.

68. The method of any one of claims 45-67, wherein the pharmaceutical composition is administered orally.

69. The method of any one of claims 45-68, wherein the pharmaceutical composition is administered immediately after, about 1 hour after, about 3 hours after, about 6 hours after, about 12 hours after, about 1 day after, about 3 days after, about 1 week after, about 2 weeks after, about 1 month after, about 2 months after, about 3 months after, about 4 months after, about 5 months after, about 6 months after, about 7 months after, about 8 months after, about 9 months after, about 10 months after, about 11 months after, or about 12 months after a surgical treatment for CD.

70. A pharmaceutical composition comprising a plurality of diastereomers of a SMAD7 antisense oligonucleotide (ASO) that is pharmacologically and/or clinically efficacious, made by a method comprising:
a) confirming a ³¹P-NMR spectrum for the plurality of diastereomers of one or more resonances between 54.8 to 55.5 ppm; and
b) confirming the ³¹P-NMR spectrum has:
(i) a first principal component 1 (PC1) score that is below about −0.20 or above about 0.25 and a second principal component 2 (PC2) score that is below about 0.00, or
(ii) a PC1 score that is outside of the range of −0.32 to 0.30 and/or a PC2 score that is outside the range of 0.00 to 0.20,
as determined by principal component analysis (PCA).

71. A method of manufacturing a pharmaceutical composition comprising a plurality of diastereomers of a SMAD7 antisense oligonucleotide (ASO), the method comprising:
a) confirming a ³¹P-NMR spectrum for the plurality of diastereomers of one or more resonances between 54.8 to 55.5 ppm; and
b) confirming the ³¹P-NMR spectrum has:
(i) a first principal component 1 (PC1) score that is below about −0.20 or above about 0.25 and a second principal component 2 (PC2) score that is below about 0.00, or
(ii) a PC1 score that is outside of the range of −0.32 to 0.30 and/or a PC2 score that is outside the range of 0.00 to 0.20,
as determined by principal component analysis (PCA).

72. The pharmaceutical composition of claim 70 or method of claim 71, wherein the method comprises confirming:
a) the PC1 score is below about −0.20 and the PC2 score is below about 0.00;
b) the PC1 score is above about 0.25 and the PC2 score is below about 0.00;
c) the PC1 score is from about −0.47 to about −0.20 and the PC2 score is from about −0.27 to about 0.00;
d) the PC1 score is from about 0.65 to about 0.9 and the PC2 score is from about −0.47 to about 0.00;
e) the PC1 score is outside of the range of −0.32 to 0.30 when the PC2 score is above 0.00 and below 0.20; and/or
f) the PC2 score is outside the range of 0.00 to 0.20 when the PC1 is above −0.32 and below 0.30.

73. The pharmaceutical composition of claim 70 or 72 or method of claim 71 or 72, wherein the method comprises confirming:
a) the ³¹P-NMR spectrum has two or more resonances between 54.8 to 55.5 ppm;
b) the ³¹P-NMR spectrum has one or more resonances at about 54.8 ppm;
c) the ³¹P-NMR spectrum has two or more resonances at about 54.8 ppm;
d) the ³¹P-NMR spectrum has one or more resonances at about 54.9 ppm;
e) the ³¹P-NMR spectrum has two or more resonances at about 54.9 ppm;
f) the ³¹P-NMR spectrum has one or more resonances at about 55.0 ppm;
g) the ³¹P-NMR spectrum has two or more resonances at about 55.0 ppm;
h) the ³¹P-NMR spectrum has one or more resonances at about 55.1 ppm;
i) the ³¹P-NMR spectrum has two or more resonances at about 55.1 ppm;
j) the ³¹P-NMR spectrum has one or more resonances at about 55.2 ppm;
k) the ³¹P-NMR spectrum has two or more resonances at about 55.2 ppm:
l) the ³¹P-NMR spectrum has one or more resonances at about 55.3 ppm;
m) the ³¹P-NMR spectrum has two or more resonances at about 55.3 ppm;
n) the ³¹P-NMR spectrum has one or more resonances at about 55.4 ppm;
o) the ³¹P-NMR spectrum has two or more resonances at about 55.4 ppm;
p) the ³¹P-NMR spectrum has one or more resonances at about 55.5 ppm;
q) the ³¹P-NMR spectrum has two or more resonances at about 55.5 ppm; and/or
r) the ³¹P-NMR spectrum has two or more resonances between 54.8 to 55.5 with different intensities.

74. A method of predicting pharmacological and/or clinical efficacy of a candidate composition comprising a plurality of diastereomers of a SMAD7 antisense oligonucleotide (ASO), the method comprising subjecting a ³¹P-NMR spectrum for the plurality of diastereomers to principal component analysis (PCA) to obtain a first principal component (PC1) and a second principal component (PC2), wherein:
a) a PC1 score that is below about −0.20 or above about 0.25 and a PC2 score that is below about 0.00 is predictive of pharmacological and/or clinical efficacy, or
b) a PC1 score that is outside of the range of −0.32 to 0.30 and/or a PC2 score that is outside the range of 0.00 to 0.20,
is predictive of pharmacological and/or clinical efficacy.

75. The method of claim 74, wherein:
a) a PC1 score that is below about −0.20 and a PC2 score that is below about 0.00;
b) a PC1 score above about 0.25 and a PC2 score that is below about 0.00;
c) a PC1 score from about −0.47 to about −0.20 and a PC2 score that is from about −0.27 to about 0.00;
d) a PC1 score from about 0.65 to about 0.9 and a PC2 score that is from about −0.47 to about 0.00;
c) a PC1 score outside of the range of −0.32 to 0.30 when the PC2 score is above 0.00 and below 0.20; and/or
f) a PC2 score is outside the range of 0.00 to 0.20 when the PC1 is above −0.32 and below 0.30,
is predictive of pharmacological and/or clinical efficacy.

76. The pharmaceutical composition of claim 70 or 72 or method of any one of claims 71-75, wherein the PCA comprises selecting principle components using a modeling data set.

77. The pharmaceutical composition of claim 76 or method of claim 76, wherein the modeling data set comprises five, six, seven, eight, nine, ten, or up to twenty-seven ³¹P-NMR spectra selected from the ³¹P-NMR spectra as shown in FIGS. 5C-5CC.

78. The pharmaceutical composition of claim 76 or 77 or method of claim 76 or 77, wherein the modeling data set comprises the ³¹P-NMR spectra as shown in FIGS. 5C, 5L, 5X, 5H, 5P, 5I, 5M, 5T, 55, and 5BB.

79. The pharmaceutical composition of any one of claims 70 and 71-78 or method of any one of claims 71-78, wherein the principal components are selected to account for more than 90% variance of the modeling data set.

80. The pharmaceutical composition of any one of claims 70 and 71-79 or method of any one of claims 71-79, wherein the ³¹P-NMR is performed in solution at about 14.1 T.

81. The pharmaceutical composition of any one of claims 70 and 71-80 or method of any one of claims 71-80, wherein the SMAD7 ASO comprises a sequence selected from any one of SEQ ID NOs: 1-6 or a pharmaceutically acceptable salt thereof.

82. The pharmaceutical composition of any one of claims 70 and 71-81 or method of any one of claims 71-81, wherein internucleotide linkages of the SMAD7 ASO are O,O-linked phosphorothioates.

83. The pharmaceutical composition of any one of claims 70 and 71-82 or method of any one of claims 71-82, wherein the SMAD7 ASO has a sequence according to SEQ ID NO: 1 (5′-GTXGCCCCTTCTCCCXGCAGC-3′) in which all internucleoside linkages are O,O-linked phosphorothioate linkages and X is 5-methyl 2′-deoxycytidine.

84. A method of treating an inflammatory bowel disease in a subject in need thereof, comprising administering to the subject the pharmaceutical composition of any one of claims 70 and 71-83, the SMAD7 ASO manufactured according to any one of claims 71-73 and 76-83 or the SMAD7 ASO predicted to be pharmacologically and/or clinically efficacious according to any one of claims 74-83.

85. The method of claim 84, wherein the inflammatory bowel disease is Crohn's disease.

86. The method of claim 85, wherein the Crohn's disease is postoperative Crohn's disease.


SEQUENCE LISTING (6 sequences)

SEQ ID NO: 1 - DNA, 21 bp, Artificial Sequence
  Description of Artificial Sequence Synthetic oligonucleotide
  modified_base (3)..(3): 5-methyl-2′-deoxycytidine
  modified_base (16)..(16): 5-methyl-2′-deoxycytidine
gtcgccccttctccccgcagc

SEQ ID NO: 2 - DNA, 21 bp, Artificial Sequence
  Description of Artificial Sequence Synthetic oligonucleotide
  modified_base (3)..(3): 5-methyl-2′-deoxycytidine
  modified_base (16)..(16): 5-methyl-2′-deoxycytidine
gtcgccccttctctccgcagc

SEQ ID NO: 3 - DNA, 20 bp, Artificial Sequence
  Description of Artificial Sequence Synthetic oligonucleotide
  See specification as filed for detailed description of substitutions and preferred embodiments
  modified_base (3)..(3): Nitrogenous base cytosine, 5-methylcytosine or 2′-O-methylcytosine
  modified_base (4)..(4): Nitrogenous base guanine, 5-methylguanine or 2′-O-methylguanine
  modified_base (16)..(16): Nitrogenous base cytosine, 5-methylcytosine or 2′-O-methylcytosine
  modified_base (17)..(17): Nitrogenous base guanine, 5-methylguanine or 2′-O-methylguanine
gtcgccccttctccccgcag

SEQ ID NO: 4 - DNA, 20 bp, Artificial Sequence
  Description of Artificial Sequence Synthetic oligonucleotide
  modified_base (3)..(3): 5-methyl-2′-deoxycytidine
  modified_base (16)..(16): 5-methyl-2′-deoxycytidine
gtcgccccttctccccgcag

SEQ ID NO: 5 - DNA, 21 bp, Artificial Sequence
  Description of Artificial Sequence Synthetic oligonucleotide
  See specification as filed for detailed description of substitutions and preferred embodiments
  modified_base (3)..(3): 5-methyl-2′-deoxycytidine
  modified_base (16)..(16): 5-methyl-2′-deoxycytidine
  modified_base (17)..(17): Nitrogenous base guanine, 5-methylguanine or 2′-O-methylguanine
gtcgccccttctccccgcagc

SEQ ID NO: 6 - DNA, 21 bp, Artificial Sequence
  Description of Artificial Sequence Synthetic oligonucleotide
  See specification as filed for detailed description of substitutions and preferred embodiments
  modified_base (3)..(3): 5-methyl-2′-deoxycytidine
  modified_base (16)..(16): 5-methyl-2′-deoxycytidine
  modified_base (17)..(17): Nitrogenous base guanine, 5-methylguanine or 2′-O-methylguanine
gtcgccccttctctccgcagc